(19) United States
(12) Patent Application Publication
(10) Pub. No.: US 2024/0269316 A1
(43) Pub. Date: Aug. 15, 2024

(54) NUCLEIC ACID SENSOR AGONIST COMPOSITIONS AND USES THEREOF

(71) Applicant: HDT Bio Corp., Seattle, WA (US)

(72) Inventors: Malcolm S. Duthie, Sammamish, WA (US); Bryan Berube, Issaquah, WA (US); Joseph Leal, Seattle, WA (US); Amit Praful Khandhar, Issaquah, WA (US); Jiho Kim, Seattle, WA (US); Darrick Albert Carter, Seattle, WA (US); Lars Peter Aksel Berglund, Seattle, WA (US)

(21) Appl. No.: 18/612,893

(22) Filed: Mar. 21, 2024

Related U.S. Application Data
(63) Continuation of application No. PCT/US2022/076821, filed on Sep. 21, 2022.
(60) Provisional application No. 63/246,880, filed on Sep. 22, 2021.
(60) Provisional application No. 63/302,150, filed on Jan. 24, 2022.

(51) Int. Cl. A61K 47/69; A61K 9/19; A61K 9/50; A61K 9/51; A61K 47/06; A61K 47/12; A61K 47/54

(52) U.S. Cl. CPC A61K 47/6907; A61K 9/19; A61K 9/5015; A61K 9/5115; A61K 47/06; A61K 47/12; A61K 47/543

(57) ABSTRACT

The disclosure provides nanoparticle compositions and methods of making and using the same to deliver a bioactive agent such as a nucleic acid which is a PRR agonist to a subject. Various nanoparticle carriers are described. In some instances, the nanoparticle component may include a hydrophobic core having an inorganic particle, and optionally a membrane having a cationic lipid. Various PRR agonists are described. In some instances, the PRR agonist is a RIG-I agonist or a TLR3 agonist.

### CROSS REFERENCE

This application is a continuation of International Application No. PCT/US2022/076821, filed Sep. 21, 2022, which claims the benefit of priority to U.S. Provisional Patent Application No. 63/246,880, filed Sep. 22, 2021, and U.S. Provisional Patent Application No. 63/302,150, filed Jan. 24, 2022, the contents of each of which are incorporated herein by reference in their entirety.

### SEQUENCE LISTING

The instant application contains a Sequence Listing which has been submitted electronically in xml format and is hereby incorporated by reference in its entirety. Said xml copy, created on Mar. 19, 2024, is named 201953-706301—SL.xml and is 15,880 bytes in size.

### BACKGROUND

Certain proteins function as nucleic acid sensors that monitor cells for unusual or foreign nucleic acids. These nucleic acid sensors can survey various cellular compartments, such as the endosomal regions (e.g., TLR3, TLR7, TLR8, and TLR9), or the cytosol (retinoic acid-inducible gene I (RIG-I)-like receptors (RLRs). Once activated, these nucleic acid sensors trigger signaling cascades resulting in activities and gene activation having anti-viral and anti-cancer activities. Certain nucleic acid sensors may be referred to as pattern-recognition receptors (PRRs). RLRs are PRRs that detect certain patterns in singled-stranded ribonucleic acid (RNA) or DNA (ssRNA, ssDNA), or double-stranded RNA (dsRNA) sequences. PRR agonists provide a mechanism for increased immune activamtion, which has potential therapeutic implications. However, PRR agonists face various therapeutic challenges, with toxicity associated with high nucleic acid dosage amount, delivery, and stability. Thus, there is a need for solutions for improved PRR agonist therapeutics for enhancing immune activity for treatment and prevention of disease conditions.

### BRIEF SUMMARY

Provided herein are compositions, wherein the compositions comprise: a nanoparticle, wherein the nanoparticle comprises: a hydrophobic core, wherein lipids present in the hydrophobic core are in liquid phase at 25 degrees Celsius; and a hydrophilic surface; and a nucleic acid, wherein the nucleic acid comprises a region encoding a sequence that is at least 85% identical to any one of SEQ ID NOS: 1-11.

Provided herein are compositions, wherein the compositions comprise: lipid nanoparticles, wherein the lipid nanoparticles are characterized as having a z-average diameter particle size measurement of 20 nm to 80 nm when measured using dynamic light scattering, and wherein the lipid nanoparticles comprise: a surface comprising cationic lipids; and a hydrophobic core, wherein the hydrophobic core comprises liquid oil, wherein lipids present in the hydrophobic core are in liquid phase at 25 degrees Celsius, and: a nucleic acid, wherein the nucleic acid comprises a pattern recognition receptor (PRR) agonist region, wherein the nucleic acid is present in an amount of up to 1 mg, and wherein the nucleic acid is in complex with the hydrophilic surface.

Provided herein are compositions, wherein the compositions comprise: a nanoparticle, wherein the nanoparticle comprises: a hydrophobic core comprising an inorganic particle; and a hydrophilic surface; and a nucleic acid, wherein the nucleic acid comprises a pattern recognition receptor (PRR) agonist region, wherein the nucleic acid is present in an amount of up to 1 mg, and wherein the nucleic acid is in complex with the hydrophilic surface.

Provided herein are compositions, wherein the compositions comprise: a nanoparticle, wherein the nanoparticle comprises: a hydrophobic core comprising an inorganic particle; and a hydrophilic surface; and a nucleic acid, wherein the nucleic acid comprises a region coding a sequence at least 85% identical to SEQ ID NO: 1.

Provided herein are suspensions, wherein the suspensions comprise a composition provided herein.

Provided herein are pharmaceutical compositions, wherein the pharmaceutical compositions comprise a composition provided herein; and a pharmaceutical excipient. Further provided herein are pharmaceutical compositions, wherein the pharmaceutical composition is formulated for intranasal administration or intratumoral administration.

Provided herein are methods for the treatment of cancer in a subject, the method comprising: administering to a subject having cancer, the composition provided herein, thereby treating the cancer in the subject.

Provided herein are methods for reducing tumor size, comprising administering to a subject having cancer, a composition provided herein.

Provided herein are methods for increasing monocyte recruitment to a cancer, comprising administering to a subject having cancer, a composition provided herein. Further provided herein are methods wherein the cancer is a solid cancer or a hematopoietic cancer. Further provided herein are methods for treatment of cancer, comprising: administering to a subject having cancer a composition, wherein the composition comprises a nanoparticle, wherein the nanoparticle comprises: a hydrophobic core comprising an inorganic particle; and a hydrophilic surface; and administering radiation to the subject. Further provided herein are methods for treatment of cancer, comprising: administering to a subject having cancer a composition, wherein the composition comprises: a nanoparticle, wherein the nanoparticle comprises: a hydrophobic core comprising an inorganic particle; and a hydrophilic surface; and a nucleic acid, wherein the nucleic acid comprises a pattern recognition receptor (PRR) agonist region; and administering radiation to the subject.

Provided herein are methods for increasing cDC1 activation in a subject, comprising administering to a subject, a composition provided herein.

Provided herein are methods for treatment of an infection, comprising administering to a subject having an infection, a composition provided herein.

Provided herein are methods for reduction of severity of an infection, comprising administering to a subject having an infection, a composition provided herein.

Provided herein are methods for reduction of prevention of an infection, comprising administering to a subject, a composition provided herein.

Provided herein are methods for reduction progression of an infection, comprising administering to a subject having an infection, a composition provided herein.

Provided herein are methods for treating cancer, comprising administering to a subject having cancer, a composition of any one of the embodiments provided herein; and administering radiation to the subject. Further provided herein are methods wherein the cancer is a solid cancer or a hematopoietic cancer. Further provided herein are methods for treatment of cancer, comprising: administering to a subject having cancer a composition, wherein the composition comprises a nanoparticle, wherein the nanoparticle comprises: a hydrophobic core comprising an inorganic particle; and a hydrophilic surface; and administering radiation to the subject. Further provided herein are methods for treatment of cancer, comprising: administering to a subject having cancer a composition, wherein the composition comprises: a nanoparticle, wherein the nanoparticle comprises: a hydrophobic core comprising an inorganic particle; and a hydrophilic surface; and a nucleic acid, wherein the nucleic acid comprises a pattern recognition receptor (PRR) agonist region; and administering radiation to the subject.

Provided herein are methods for increasing monocyte recruitment to augment an immune response in a subject, optionally for treatment or prevention of cancer or an infection, the method comprising: intratumorally administering to a subject the composition provided herein, thereby increasing monocyte recruitment to augment an immune response in a subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1R show schematic representations of exemplary nanoparticle (NP) carriers. FIG. 1A shows an oil-in-water emulsion. FIG. 1B shows a nanostructured lipid carrier (NLC). FIG. 1C shows a nanoparticle having an inorganic nanoparticle in liquid oil. FIGS. 1D and 1M show a nanoparticle having a cationic lipid membrane, an inorganic nanoparticle, a liquid oil core and a nucleic acid. FIG. 1E shows an oil-in-water emulsion with two or more RNA or DNA molecules. FIG. 1F shows a nanostructured lipid carrier (NLC) with two or more RNA or DNA molecules. FIG. 1G shows a nanoparticle having an inorganic nanoparticle in liquid oil two or more RNA or DNA molecules. FIGS. 1H and 1N show a nanoparticle having a cationic lipid membrane, inorganic particles, a liquid oil core, and two or more RNA or DNA molecules. FIGS. 11 and 10 show a nanoparticle having a cationic lipid membrane, a liquid oil core (e.g., squalene), and a single nucleic acid molecule. FIGS. 1J and 1P show a nanoparticle having a cationic lipid membrane, a liquid oil core (e.g., squalene), and two or more RNA or DNA molecules. FIGS. 1K and 1Q show a nanoparticle having a cationic lipid membrane, a solid core (e.g., glyceryl trimyristate-dynasan), and a single nucleic acid molecule. FIGS. 1L and 1R show a nanoparticle having a cationic lipid membrane, a solid core (e.g., glyceryl trimyristate-dynasan), and two or more RNA or DNA molecules. Drawings not to scale.

FIG. 2 shows the time measurements of nanoparticle size as measured by dynamic light scattering. X-axis is weeks and Y-axis is nm diameter. Three-time courses correspond to storage at 4, 25, and 42 degrees Celsius.

FIG. 3 depicts an image capture of an RNA electrophoresis gel. Lanes represent extracts of RIG-I agonist nucleic acid having SEQ ID NO: 2 following complex with NP-1 and treatment with or without RNase. The N:P ratio of the mixes of the RIG-I agonist to NP-1 are 25:1, 5:1, 1:1 and 0.2:1, respectively. A control lane shows results from similar treatments of uncomplexed RIG-I agonist.

FIG. 4A shows a graph measuring an indicator of IFN-β activation measured at OD635 from the supernatant of A549-Dual cells transfected with nanoparticle RNA-NP-1 complexes having varying amounts of RNA and varying N:P ratios. Horizontal lines label reference levels in untreated cells (Media).

FIG. 4B shows a graph measuring an indicator of IFIT2 activation measured in relative light units (RLU) from the supernatant of A549-Dual cells transfected with nanoparticle RNA-NP-1 complexes having varying amounts of RNA and varying N:P ratios. Horizontal lines label reference levels in untreated cells (Media).

FIG. 5A shows a graph measuring indicators of INF-β activation and IFIT2 activation from the supernatant of A549-Dual cells transfected with nanoparticle RNA-NP-1 complexes having varying amounts of RNA and varying N:P ratios, where the RNA in the complex is 0.39 ng. Horizontal lines label reference levels in untreated cells (Media).

FIG. 5B shows a graph measuring indicators of IFN-β activation and IFIT2 activation from the supernatant of A549-Dual cells transfected with nanoparticle RNA-NP-1 complexes having varying amounts of RNA and varying N:P ratios, where the RNA in the complex is 1.6 ng. Horizontal lines label reference levels in untreated cells (Media).

FIG. 6A shows a graph of particle size measurements of RIG-I agonist RNA in complex with NP-1 of N:P 8:1 and stored at the indicated temperature for 28 days. Dash lines represent NP-1 alone.

FIG. 6B shows a graph of PDI measurements of RIG-I agonist RNA in complex with NP-1 at N:P 8:1 and stored at the indicated temperature for 28 days. Dash lines represent NP-1 alone.

FIGS. 7A-7B show RNA electrophoresis gels taken from supernatant of A549-Dual cells that were transfected with RNA-NP-1 complexes measured at timepoints 0, 1, 2, 4, and 7 days, using the compositions at various storage conditions. FIG. 7A shows an RNA electrophoresis gel at 25 degrees Celsius. FIG. 7B shows an RNA electrophoresis gel at 42 degrees Celsius.

FIGS. 8A-8B show graphs of IFIT2 activation measured in relative light units from the supernatant of A549-Dual cells transfected with nanoparticle RNA-NP-1 complexes having N:P ratio of 8:1, with timepoints 0, 1, 2, 4, and 7 days, where storage conditions at −20, 4, 25, and 42 degrees Celsius. Samples were compared to media only controls and a fresh RNA-NP-1 complexes prepared the day of the assay. FIG. 8A shows results at 25 degrees Celsius. FIG. 8B shows results at 42 degrees Celsius.

FIGS. 8C-8D show graphs of the quantification of indicators of IFN-β activation measured at OD 635 from the supernatants of A549-Dual cells transfected with RNA-NP-1 complexes having N:P ratio of 8:1, measured at timepoints 0, 1, 2, 4, and 7 days, where storage conditions at −20, 4, 25, and 42 degrees Celsius. Samples were compared to media only controls and a fresh RNA-NP-1 complexes prepared the day of the assay. FIG. 8C shows results at 25 degrees Celsius. FIG. 8D shows results at 42 degrees Celsius.

FIG. 9 shows a graph of IFIT2 activation measured in relative light units from the supernatant of A549-Dual cells transfected with nanoparticle RNA-NP-1 complexes having N:P ratio of 8:1, with timepoints of 0.5 months, 1 month, 2 months, 3 months, and 6 months. All compositions were stable at 25 degrees Celsius for up to 6 months (arrow).

FIG. 10A is a bar graph showing IFIT2 activation measured in relative light units from the supernatant of A549-Dual cells treated as follows: (1) media control, (2) RIG-I RNA agonist having SEQ ID NO: 2 complexed with NP-1; (3) IFN-α Leuk; (4) IFN-α Lymph; (5) IFN-β; or (6) TNF-α. WT and RIG-I KO cells were assayed.

FIG. 10B is a bar graph of IFN-β activation measured at OD 635. A549-Dual cells treated as follows: (1) media control, (2) RIG-I RNA agonist having SEQ ID NO: 2 complexed with NP-1; (3) IFN-α Leuk; (4) IFN-α Lymph; (5) IFN-β; or (6) TNF-α. WT and RIG-I KO cells were assayed.

FIG. 11A is a bar graph showing IFIT2 activation following addition of Riboxxim alone or Riboxxim:NP-1 complexes to A549-Dual cells at the indicated Riboxxim doses (6.3, 12.5, 25, 50 and 100 ng).

FIG. 11B is a bar graph showing IFN-β activation following addition of Riboxxim alone or Riboxxim:NP-1 complexes to A549-Dual cells at the indicated Riboxxim doses (6.3, 12.5, 25, 50 and 100 ng).

FIG. 12 is bar graph showing total counts of monocytes (Y axis) from (1) untreated, (2) RIG-I RNA agonist having SEQ ID NO: 2 at 0.1 μg, (3) RIG-I RNA agonist having SEQ ID NO: 2 at 0.1 μg complexed with NP-1, (4) RIG-I RNA agonist having SEQ ID NO: 2 at 1 μg, and (5) RIG-I RNA agonist having SEQ ID NO: 2 at 1 μg complexed with NP-1.

FIG. 13A is a bar graph of flow cytometry quantification of the frequency of XCR1+CD11b-LN-Resident cDC1s, gated on CD3—B220—NK1.1-CD64-CD11c+ MHC-II int single cells (Y axis) for conditions of (1) untreated, (2) RIG-I RNA agonist having SEQ ID NO: 2 at 0.1 μg, (3) RIG-I RNA agonist having SEQ ID NO: 2 at 0.1 μg complexed with NP-1, (4) RIG-I RNA agonist having SEQ ID NO: 2 at 1 μg, and (5) RIG-I RNA agonist having SEQ ID NO: 2 at 1 μg complexed with NP-1.

FIG. 13B is a bar graph of flow cytometry quantification of the frequency of CCR7+LN-resident cDC1s, indicating cell activation (Y axis) for conditions of (1) untreated, (2) RIG-I RNA agonist having SEQ ID NO: 2 at 0.1 μg, (3) RIG-I RNA agonist having SEQ ID NO: 2 at 0.1 μg complexed with NP-1, (4) RIG-I RNA agonist having SEQ ID NO: 2 at 1 jig, and (5) RIG-I RNA agonist having SEQ ID NO: 2 at 1 μg complexed with NP-1.

FIG. 13C is a bar graph of flow cytometry quantification of the frequency of CD80+CD86++LN-resident cDC1s, indicating co-stimulatory molecule expression, (Y axis)) for conditions of (1) untreated, (2) RIG-I RNA agonist having SEQ ID NO: 2 at 0.1 μg, (3) RIG-I RNA agonist having SEQ ID NO: 2 at 0.1 μg complexed with NP-1, (4) RIG-I RNA agonist having SEQ ID NO: 2 at 1 μg, and (5) RIG-I RNA agonist having SEQ ID NO: 2 at 1 μg complexed with NP-1.

FIG. 13D is a bar graph of flow cytometry quantification of frequency of CCR7+ splenic dendritic cells (Y axis)) for conditions of (1) untreated, (2) RIG-I RNA agonist having SEQ ID NO: 2 at 0.1 μg, (3) RIG-I RNA agonist having SEQ ID NO: 2 at 0.1 μg complexed with NP-1, (4) RIG-I RNA agonist having SEQ ID NO: 2 at 1 μg, and (5) RIG-I RNA agonist having SEQ ID NO: 2 at 1 μg complexed with NP-1.

FIG. 13E is a bar graph of flow cytometry quantification of monocyte recruitment in the draining lymph node, as a percentage of total lymphocytes, under treatment conditions of: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1.

FIG. 13F is a bar graph of flow cytometry quantification of monocyte recruitment in the draining lymph node, measuring the absolute cell count, under treatment conditions of: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1.

FIG. 13G is a bar graph of flow cytometry quantification of frequency of XCR1+CD11b-LN-resident cDC1s, gated on CD3-B220-NK1.1-CD64-CD11c+ MHC-II int single cells, under treatment conditions of: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1.

FIG. 13H is a bar graph of flow cytometry quantification of the frequency of CCR7+LN-resident cDC1s, under treatment conditions of: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1.

FIG. 131 is a bar graph of flow cytometry quantification of the frequency of CD80+CD86++LN-resident cDC1s, under treatment conditions of: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1.

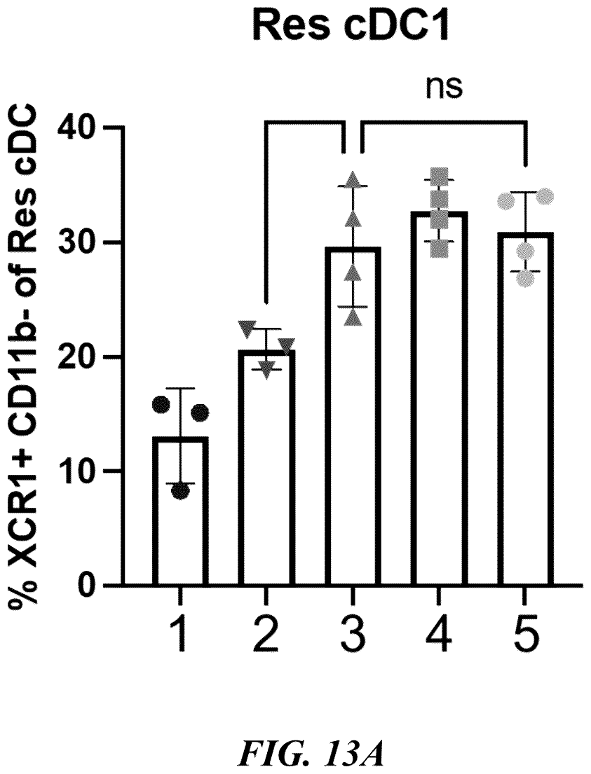

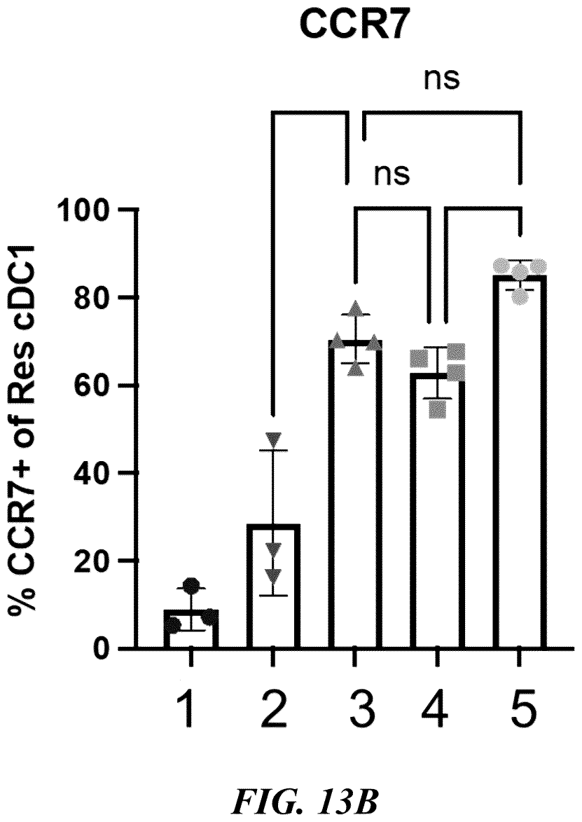

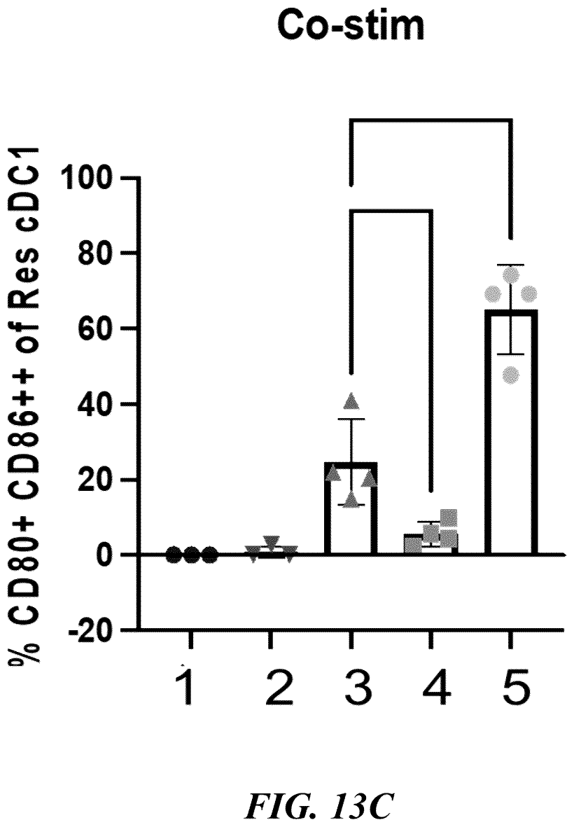

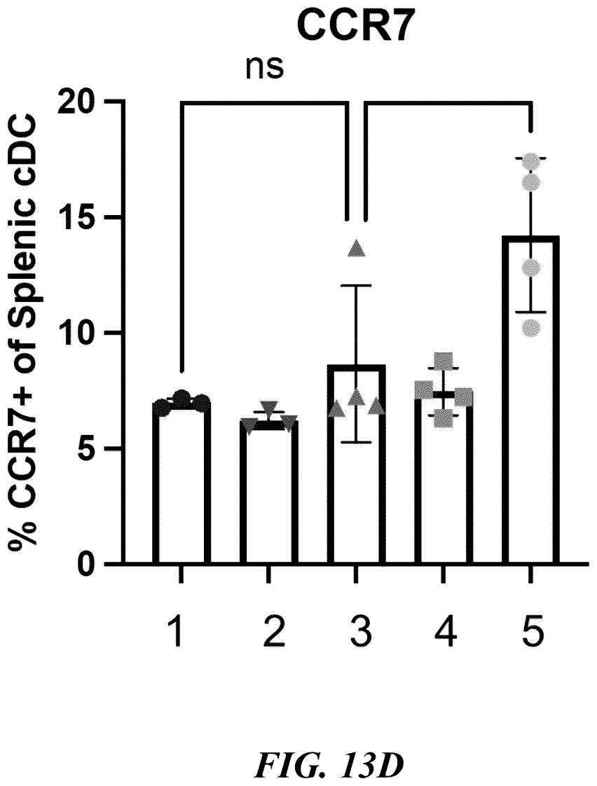

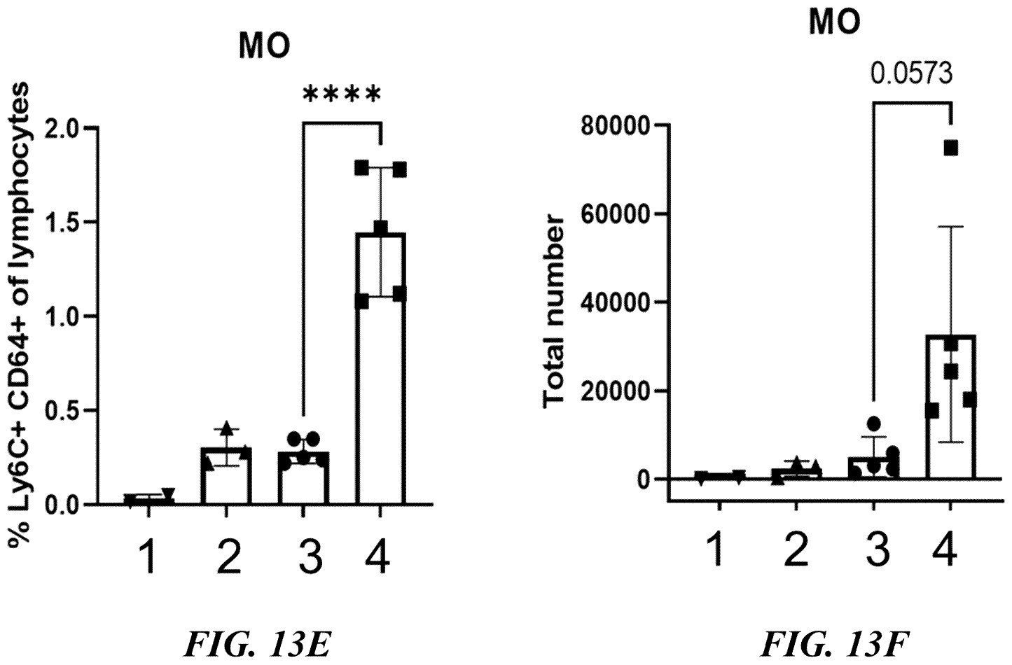

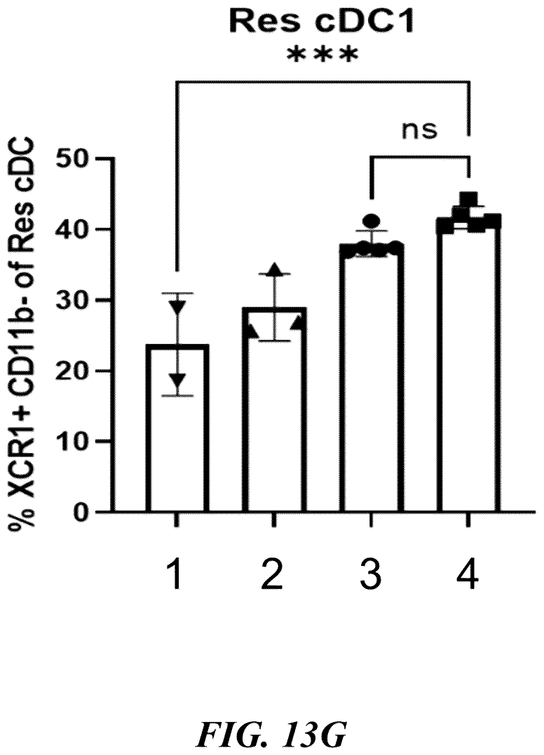

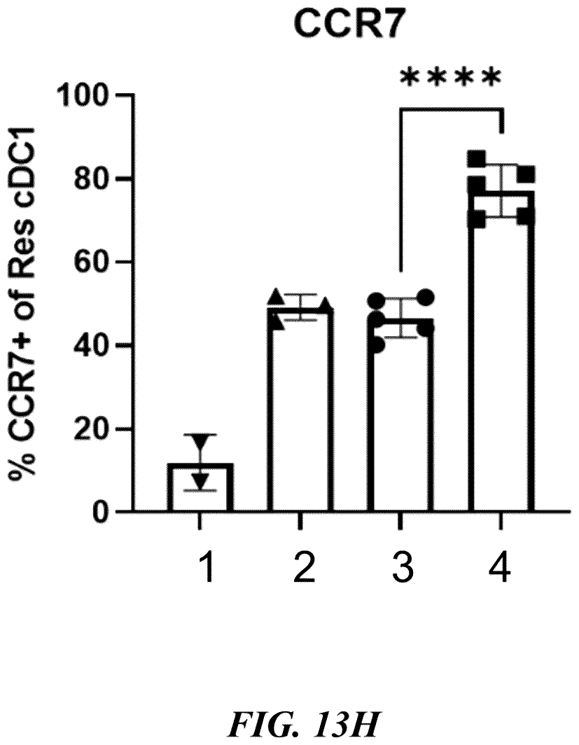

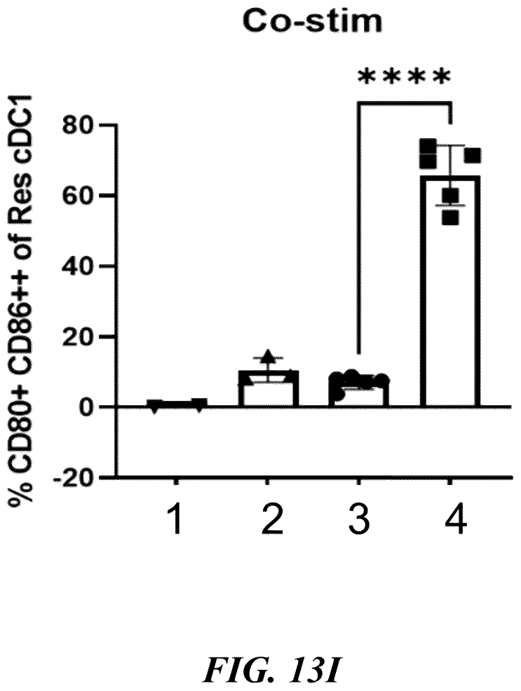

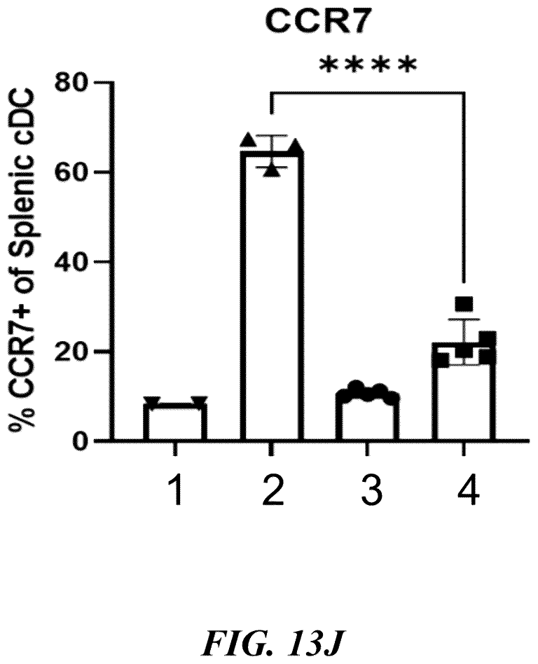

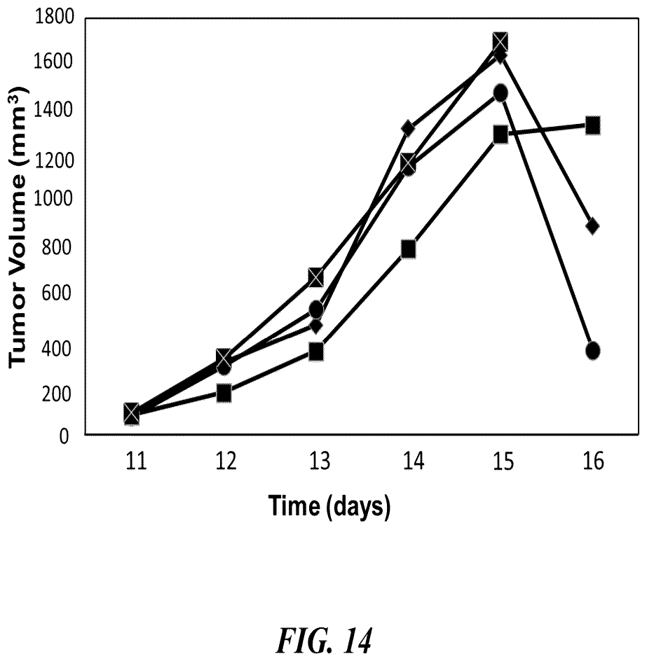

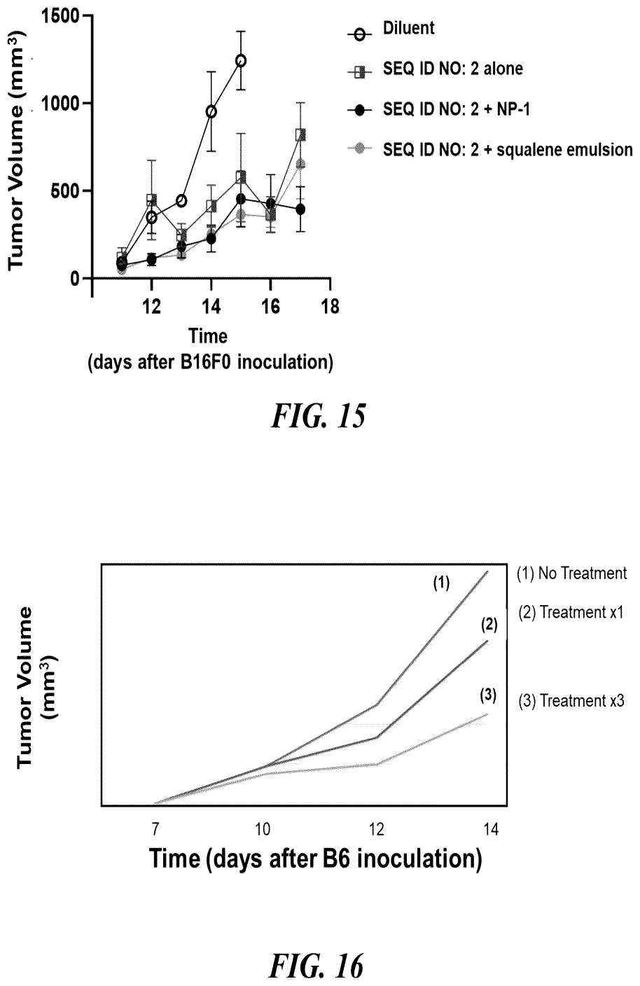

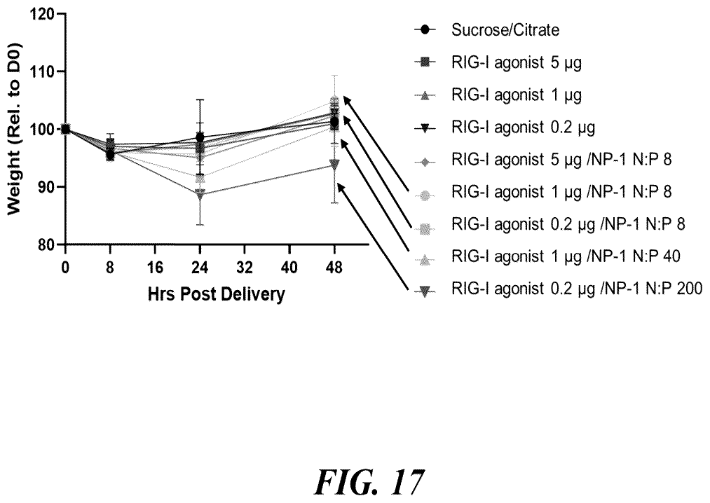

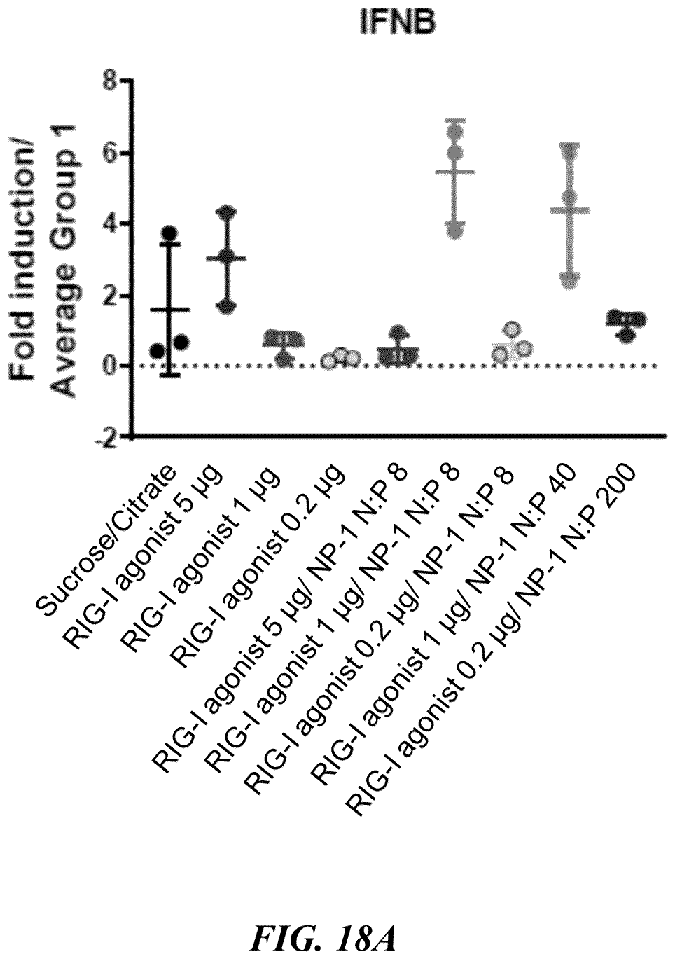

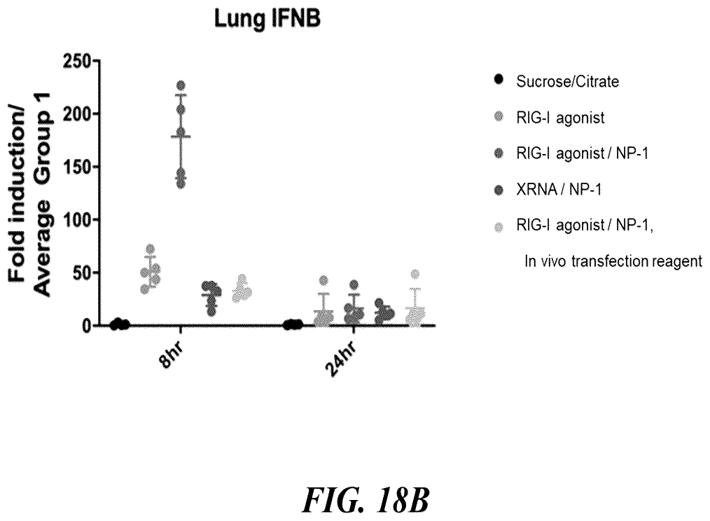

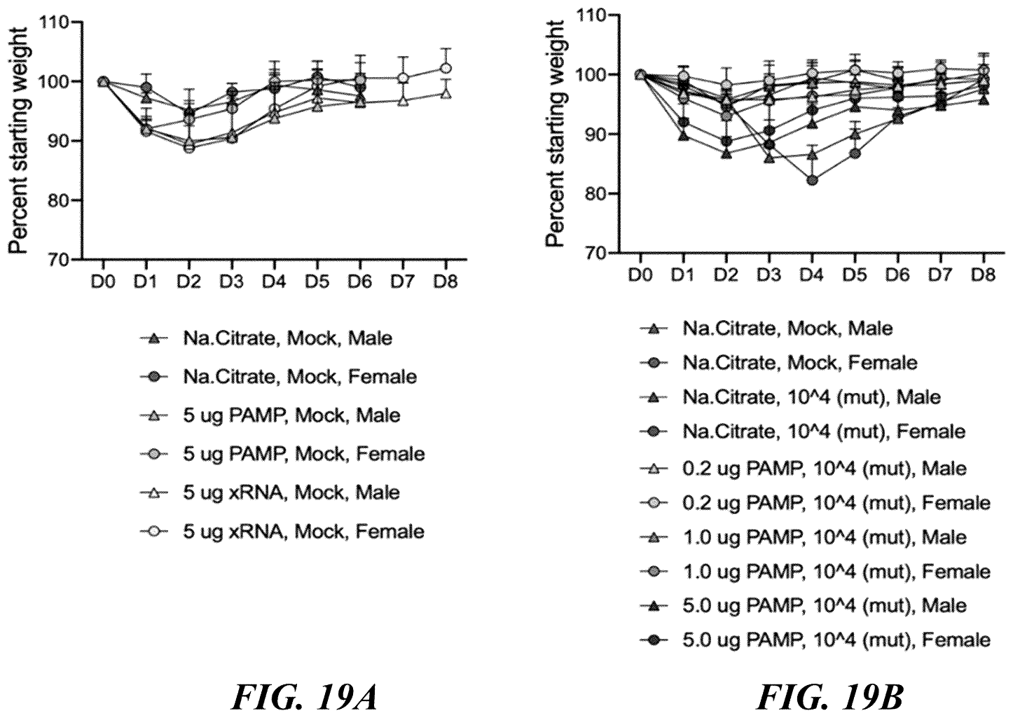

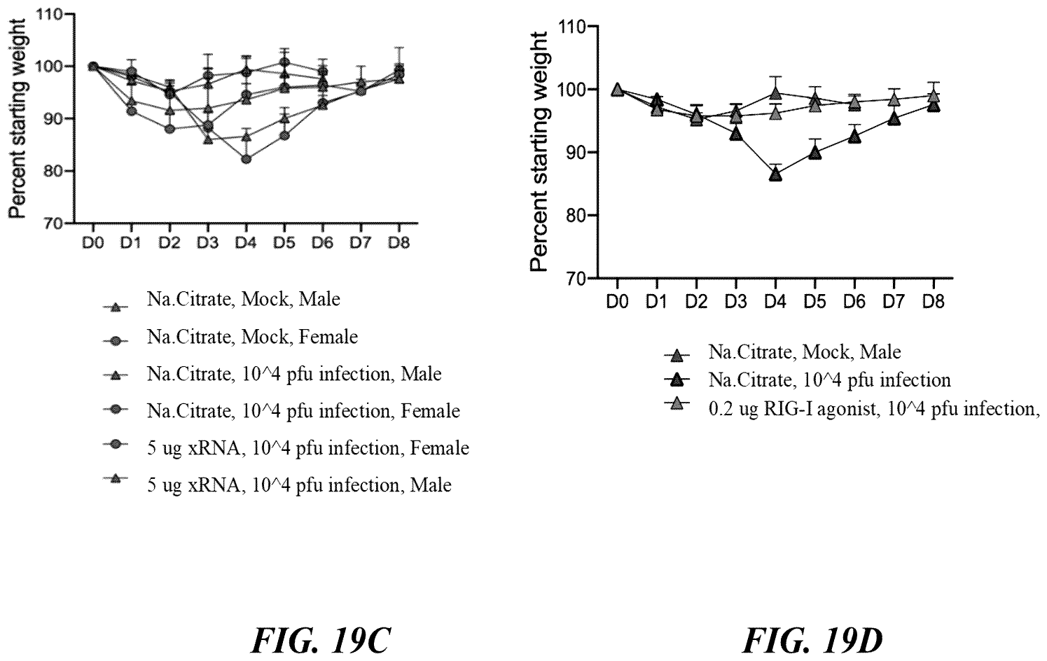

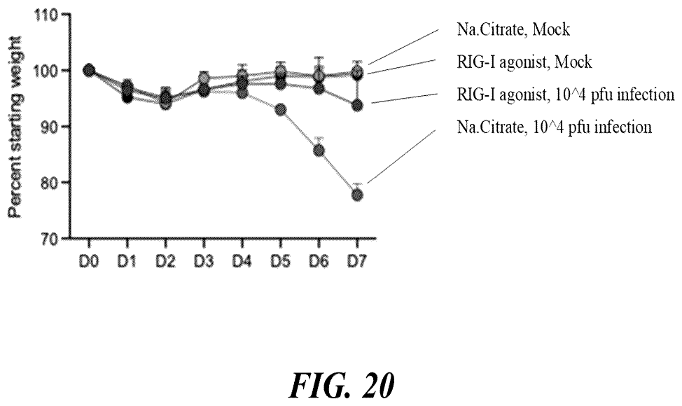

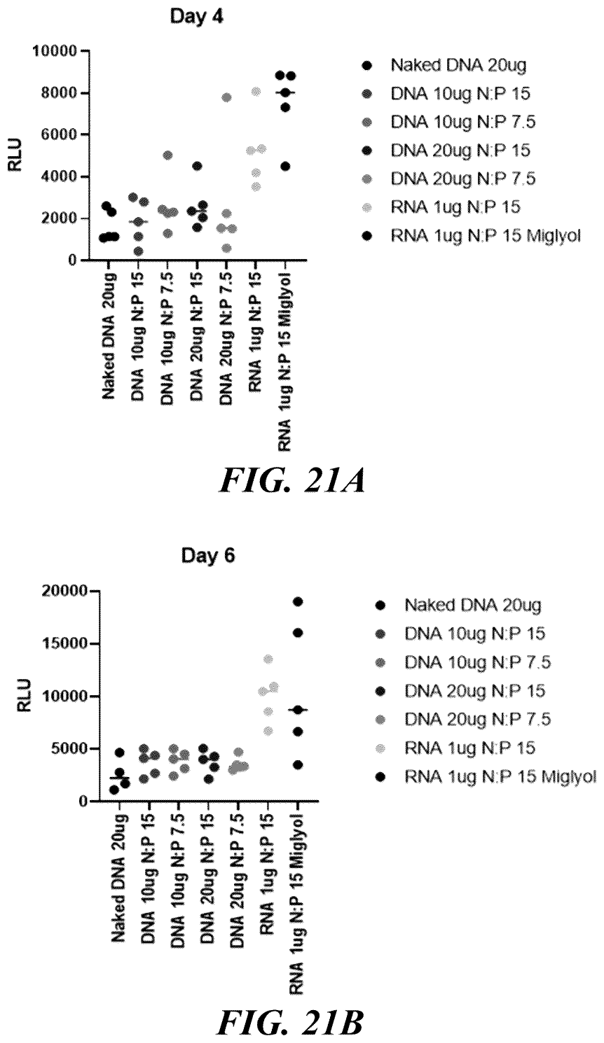

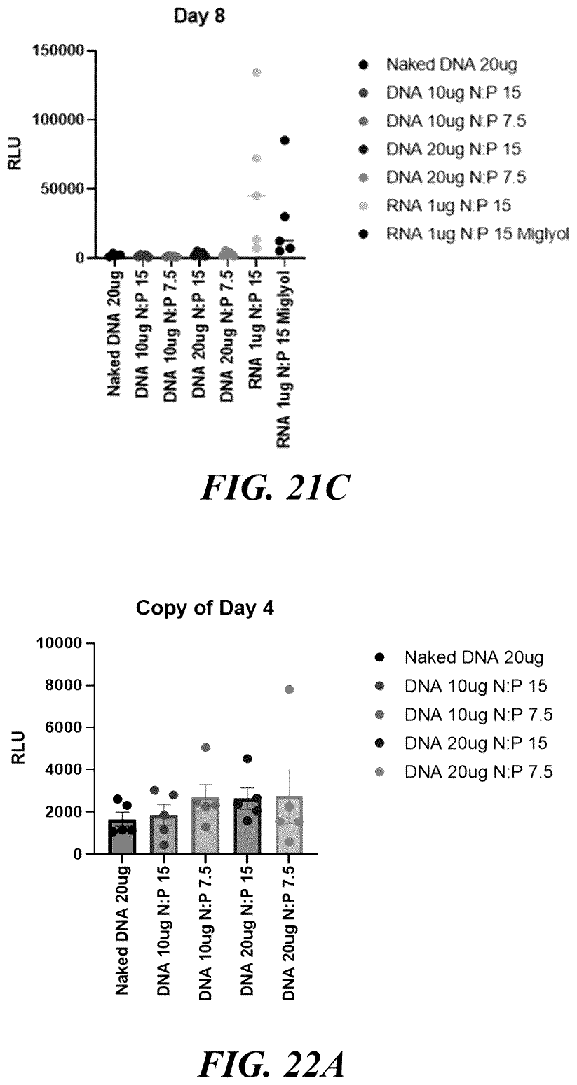

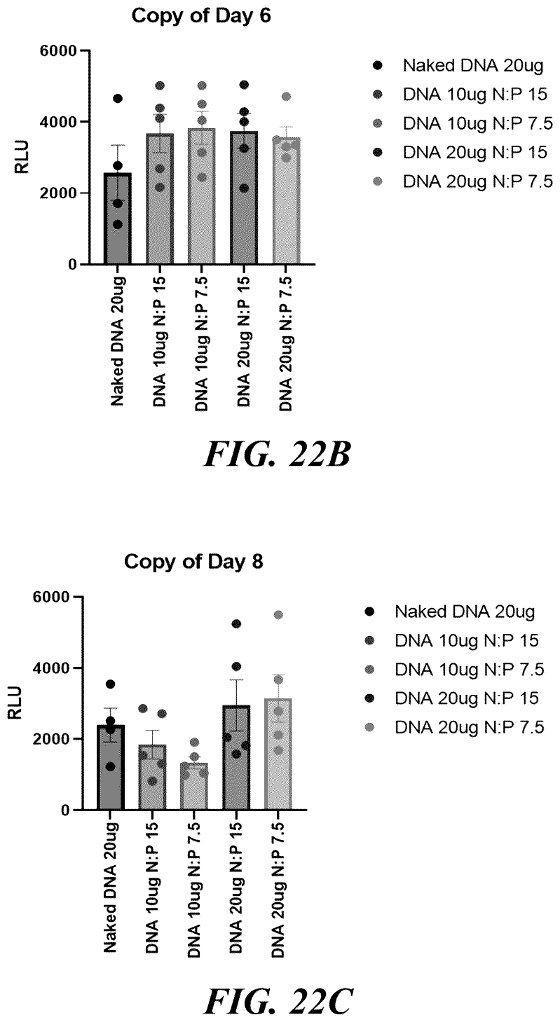

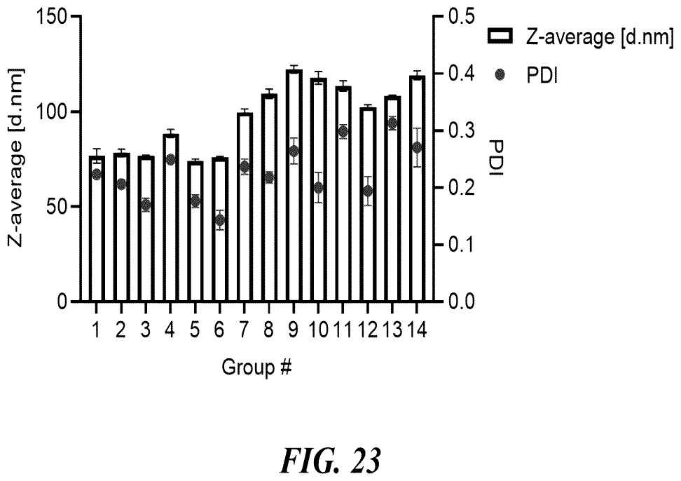

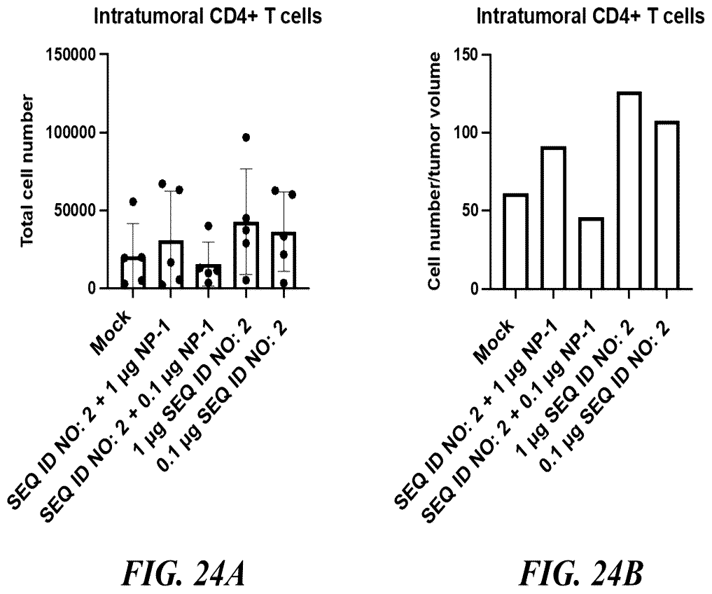

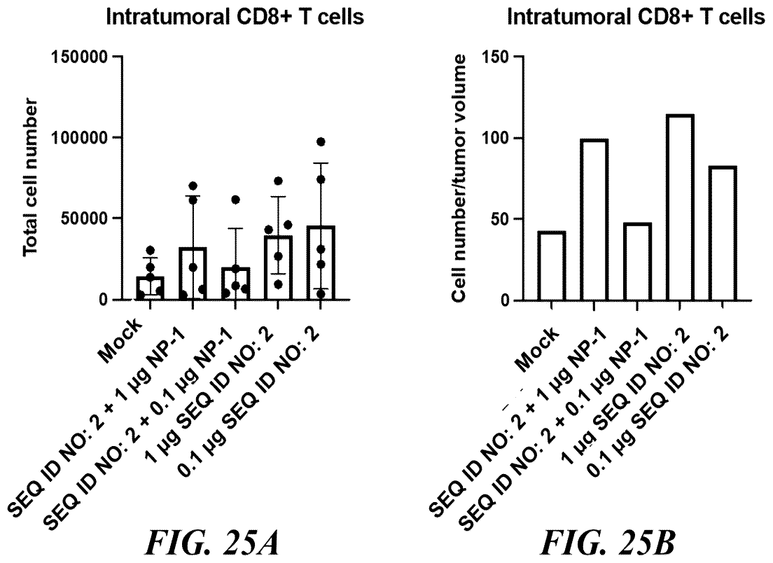

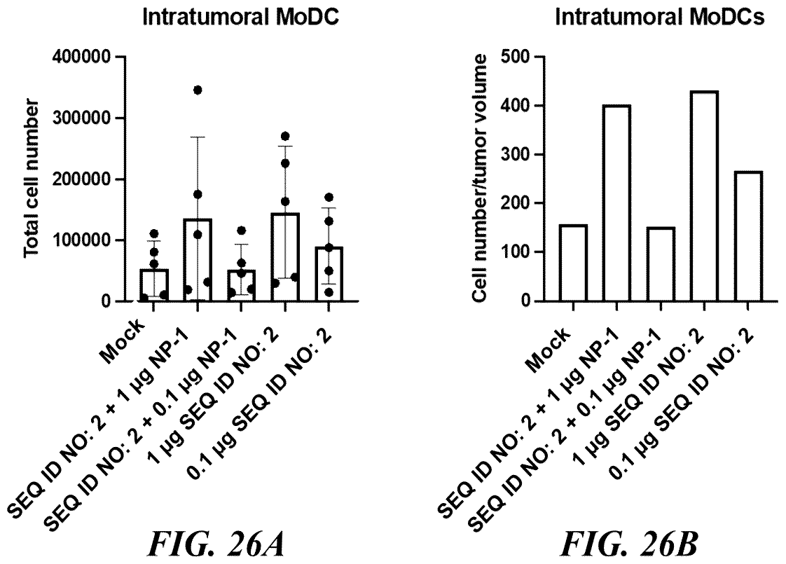

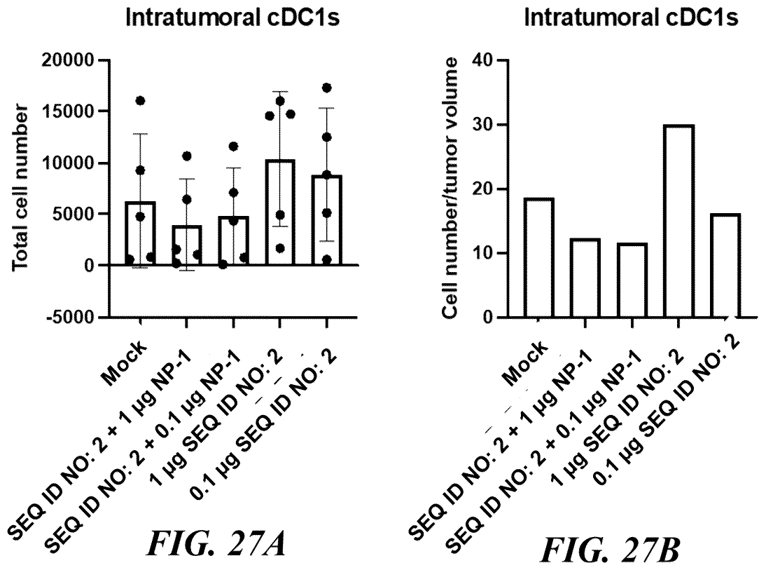

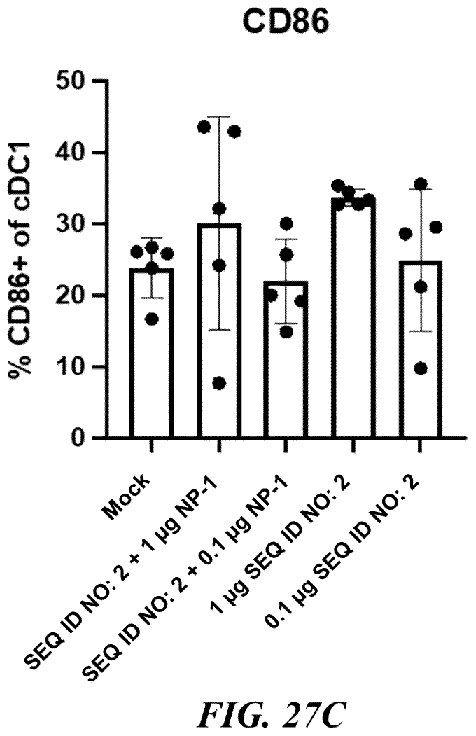

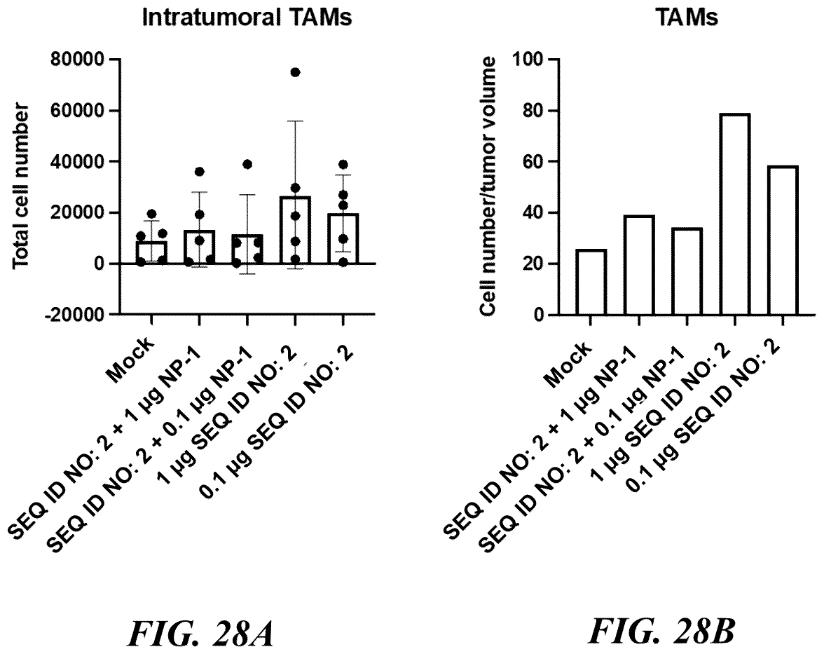

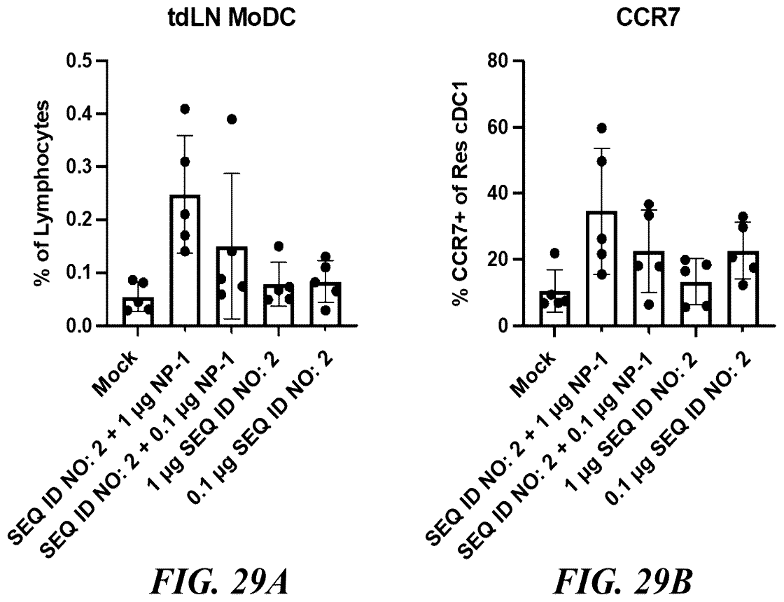

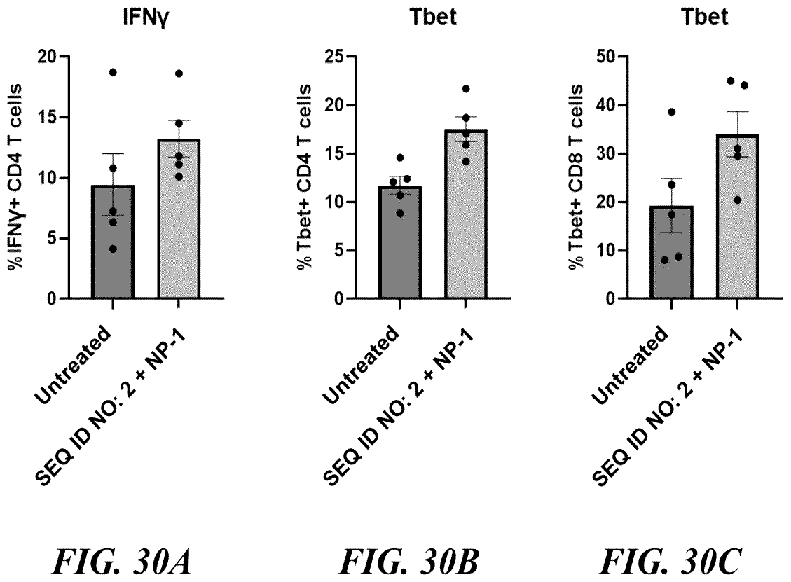

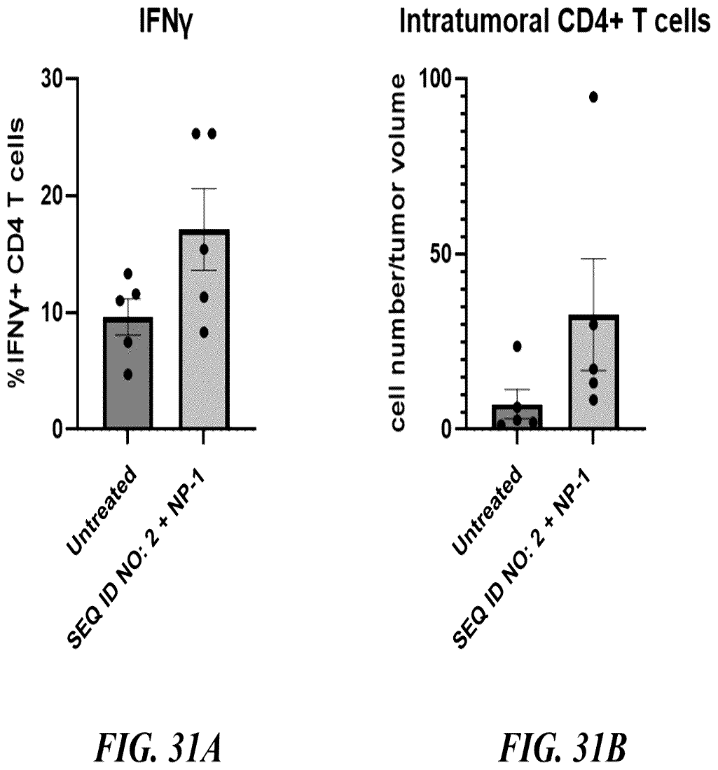

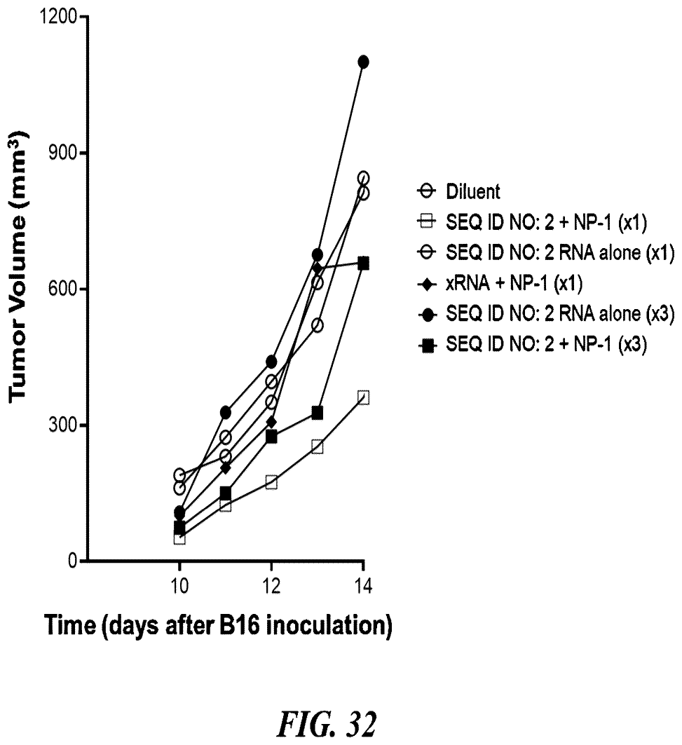

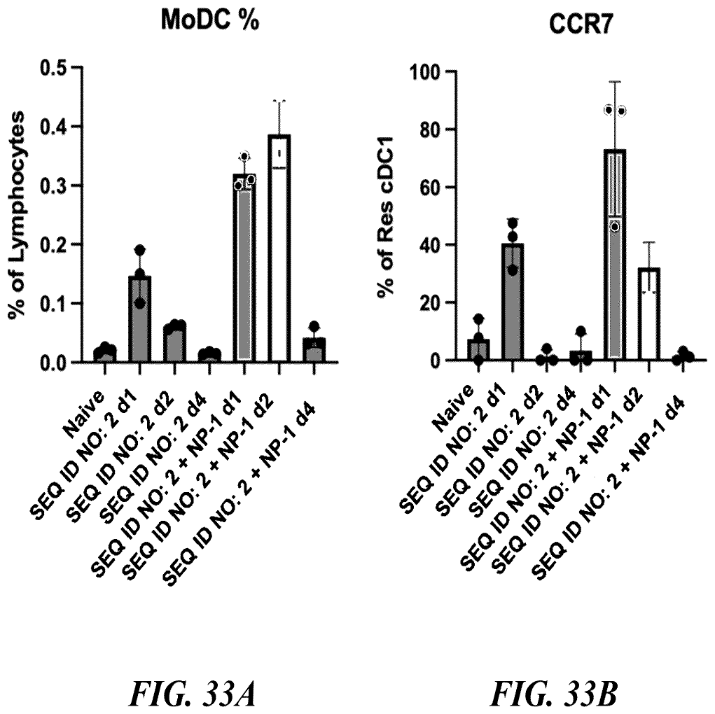

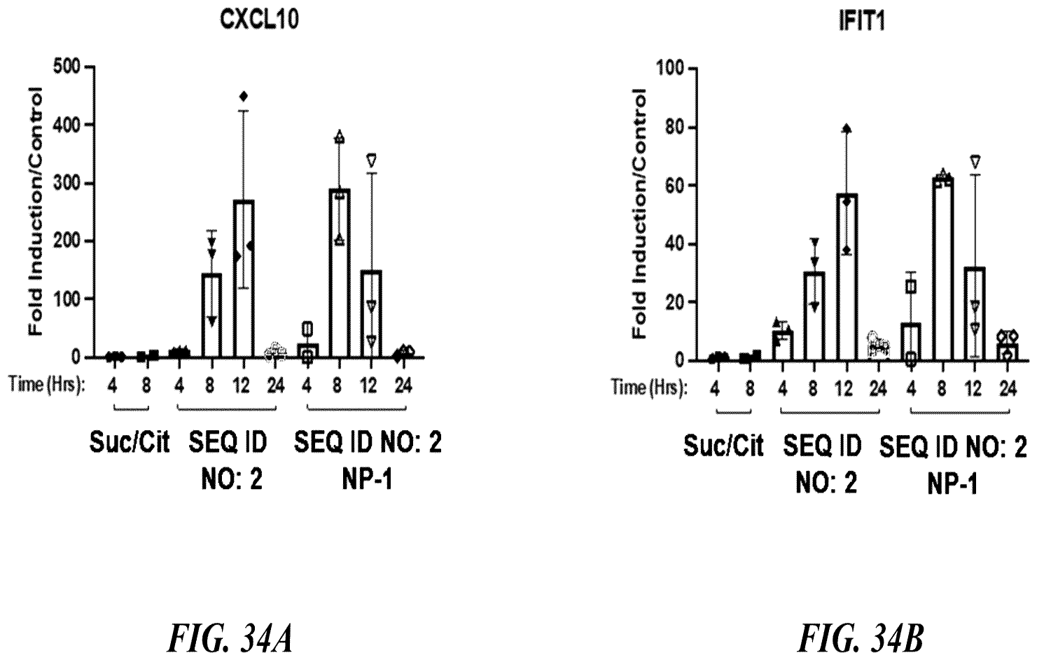

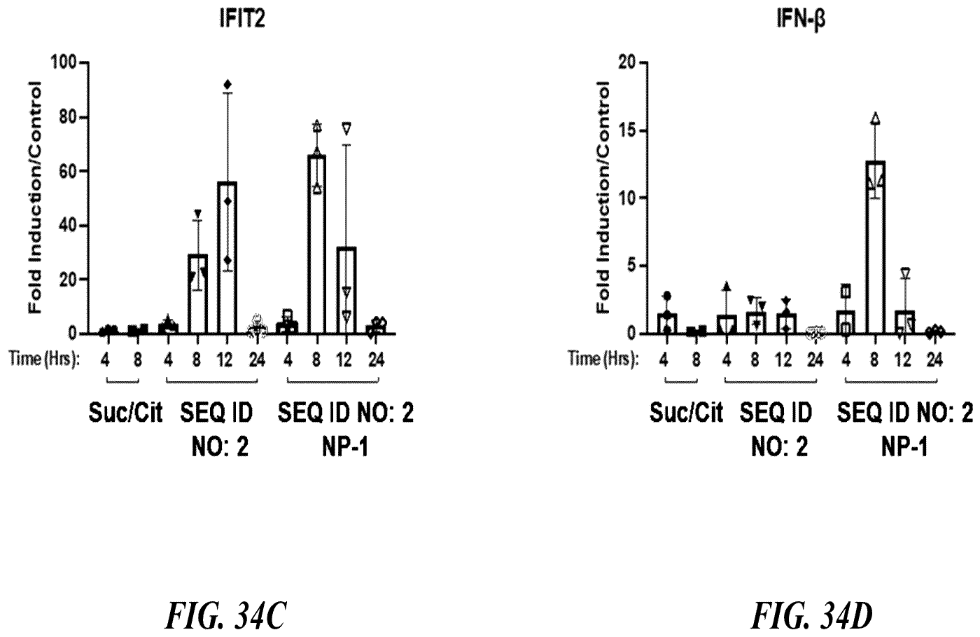

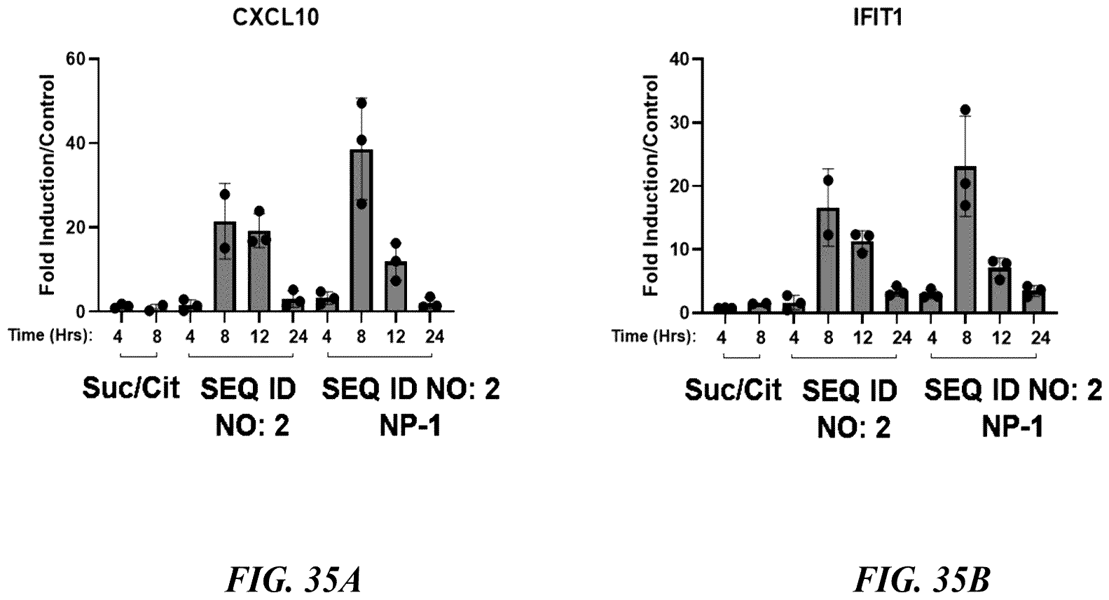

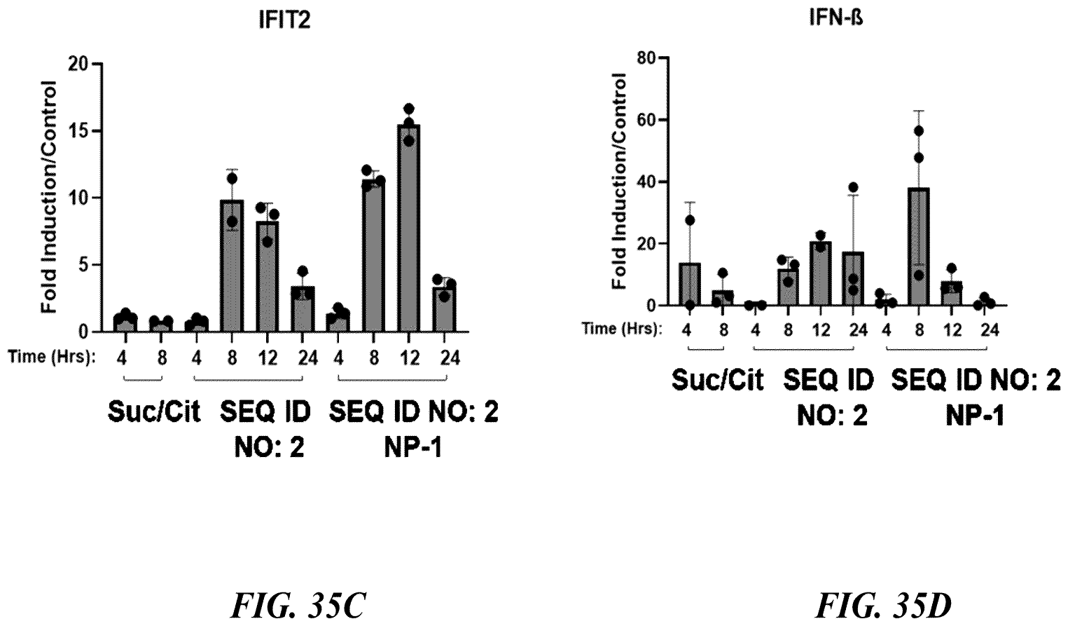

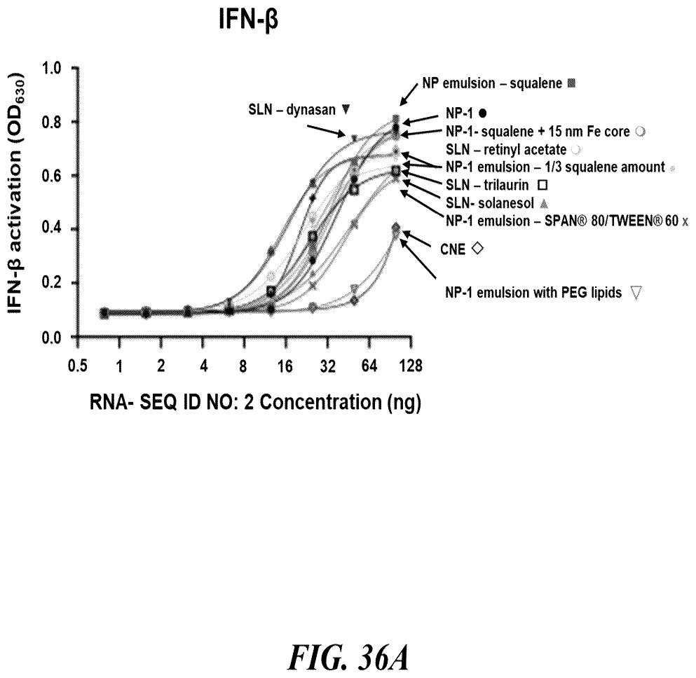

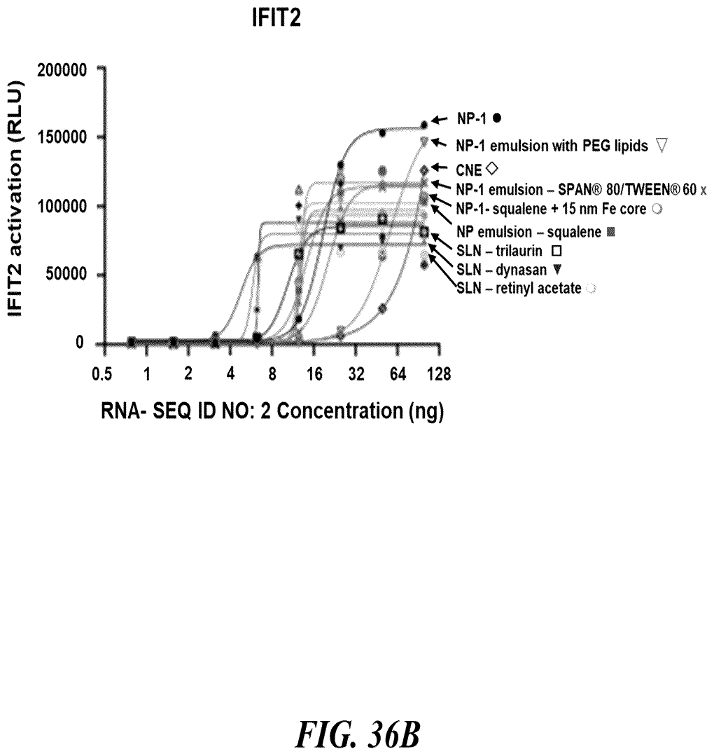

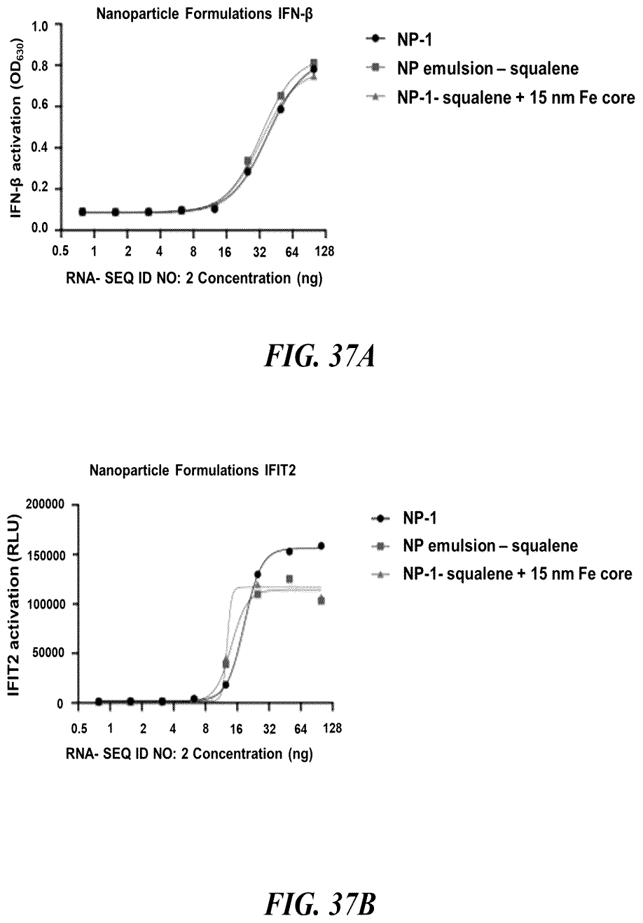

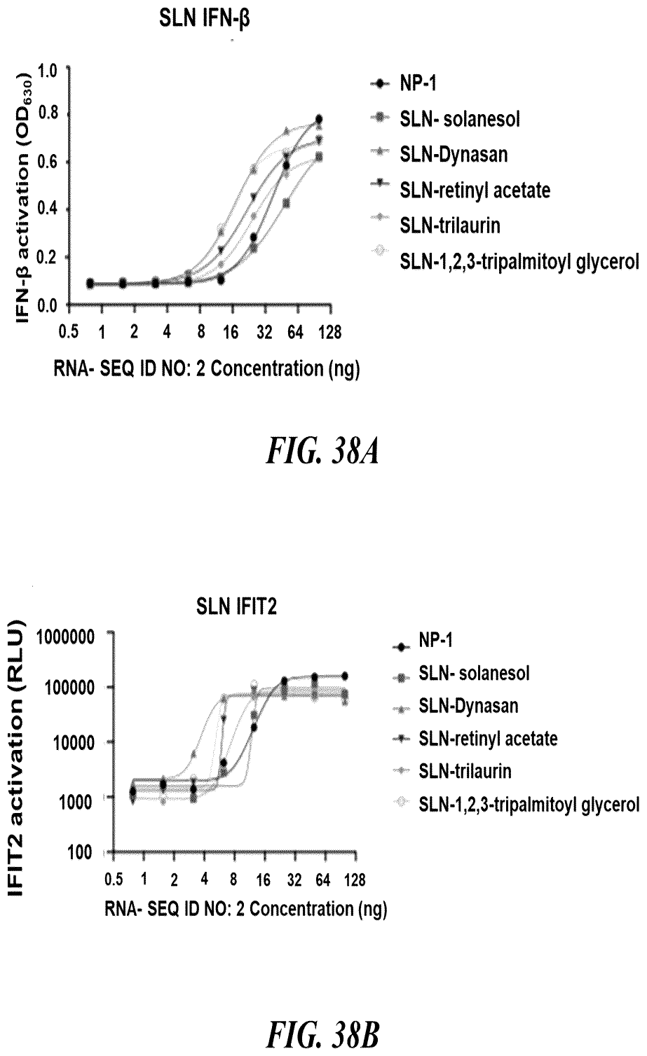

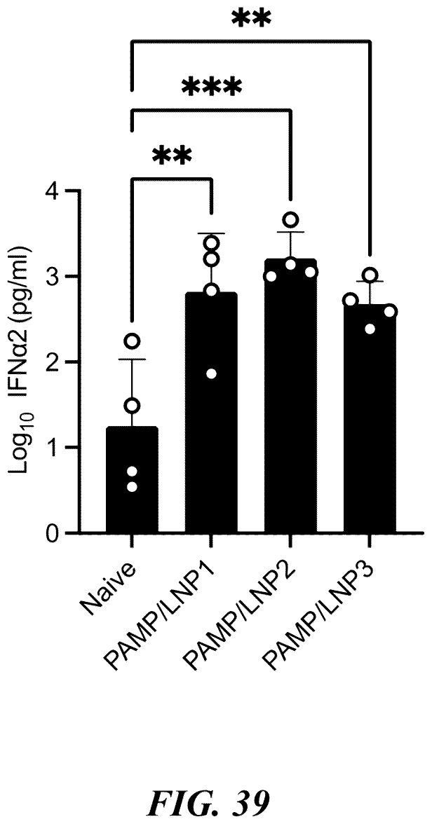

FIG. 13J is a bar graph of flow cytometry quantification of the frequency of CCR7+ splenic dendritic cells, under treatment conditions of: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1.

FIG. 14 is a line graph of tumor volume in mm³ (Y-axis) versus time in days (X-axis). Results are presented as mean for each group (1-diluent; 2-RNA agonist alone; 3-RNA agonist-NP-1 complex; 4-RNA agonist-SE complex).

FIG. 15 is a line graph of tumor volume in mm³ (Y-axis) versus time in days (X-axis). Results are presented as mean for each group (diluent; RNA agonist alone; RNA agonist-NP-1 complex; RNA agonist-SE complex).

FIG. 16 is a line graph of tumor volume (Y-axis) and days (X-axis) measurements from mice administered treatments (none, 1 time, or 3 times).

FIG. 17 is a line graph of weight (relative to day 0) on Y axis versus hours post-delivery on X axis.

FIG. 18A is a graph of fold induction of IFN-β/Average of the sucrose/citrate control (Y axis) v. time on X axis.

FIG. 18B is a graph of fold induction of IFN-β/Average of the sucrose/citrate control (Y axis) v. time on X axis.

FIG. 19A is a line graph showing percent of starting weight on the Y axis and day on the X axis, under conditions listed in control mice.

FIG. 19B is a line graph showing percent of starting weight on the Y axis and day on the X axis, under conditions listed in mice challenged with 10⁴ PFU CoV2 MA10.

FIG. 19C is a line graph showing percent of starting weight on the Y axis and day on the X axis, under conditions listed in mice challenged with 10⁴ PFU CoV2 MA10.

FIG. 19D is a line graph showing percent of starting weight on the Y axis and day on the X axis, under conditions listed in mice challenged with 10⁴ PFU CoV2 MA10.

FIG. 20 is a line plot of percent starting weight (Y axis) and days for human ACE2 transgenic mice under the conditions listed. The indicated mice were infected with 10⁴ PFU 2019-nCoV/USA-WA1/2020.

FIGS. 21A-21C are graphs of relative light units (RLU) (Y axis) measured for various injection conditions of DNA or RNA mixed with various nanoparticle conditions, at days 4, 6 and 8 post inoculation.

FIGS. 22A-22C are graphs of relative light units (RLU) (Y axis) measured for various injection conditions of DNA or RNA mixed with various nanoparticle conditions, at days 4, 6 and 8 post inoculation.

FIG. 23 is a bar chart with measurements of Z-average measurement and polydispersity index (PDI) on the Y-axis and group number on the X-axis for conditions 1 to 14.

FIG. 24A shows a graph of intratumoral CD4+ T cell number. Y-axis: total cell number. X-axis: Condition.

FIG. 24B shows a graph of intratumoral CD4+ T cell number as a function of tumor volume. Y-axis: Cell number/tumor volume. X-axis: Condition.

FIG. 25A shows a graph of intratumoral CD8+ T cell number. Y-axis: total cell number. X-axis: Condition.

FIG. 25B shows a graph of intratumoral CD8+ T cell number as a function of tumor volume. Y-axis: Cell number/tumor volume. X-axis: Condition.

FIG. 26A shows a graph of intratumoral monocyte-derived dendritic cell (MoDC) number. Y-axis: total cell number. X-axis: Condition.

FIG. 26B shows a graph of intratumoral monocyte-derived dendritic cell (MoDC) number as a function of tumor volume. Y-axis: Cell number/tumor volume. X-axis: Condition.

FIG. 27A shows a graph of intratumoral conventional dendritic cell (cDC) number.

Y-axis: total cell number. X-axis: Condition.

FIG. 27B shows a graph of intratumoral conventional dendritic cell (cDC) number as a function of tumor volume. Y-axis: Cell number/tumor volume. X-axis: Condition.

FIG. 27C shows a graph of intratumoral CD86 expression in cDC1s. Y-Axis: % CD86 of cDC1. X-axis: Condition.

FIG. 28A shows a graph of intratumoral tumor-associated macrophage (TAM) number. Y-axis: Cell number/tumor volume. X-axis: Condition.

FIG. 28B shows a graph of intratumoral tumor-associated macrophage (TAM) number as a function of tumor volume. Y-axis: Cell number/tumor volume. X-axis: Condition.

FIGS. 29A-29B show graphs of myeloid cell activation in tumor-draining lymph nodes (tdLNs) and CCR7 expression. FIG. 29A shows a graph of the % of monocyte-derived dendritic cells in the tdLN for each condition. X-axis: Condition. Y-Axis: % of lymphocytes. FIG. 29B shows a graph of CCR7 expression in cDCs. X-axis: Conditions. Y-axis: % CCR7 of cDC1. Results are presented as mean and SEM for each group, n=5.

FIGS. 30A-30C shows graphs of interferon and T-box expressed in T cells (T-bet) expression in CD4 and CD8 T cells isolated from tumor draining lymph node. FIG. 30A shows interferon-gamma (IFNγ) expression in untreated and SEQ ID NO: 2/NP-1 treated CD4+ T cells. X-Axis: conditions. Y-Axis: % IFNγ CD4+ T cells. FIG. 30B shows T-bet expression in untreated and SEQ ID NO: 2/NP-1 treated CD4+ T cells. X-Axis: conditions. Y-Axis: % T-bet CD4+ T cells.

FIG. 30C shows T-bet expression in untreated and SEQ ID NO: 2/NP-1 treated CD8+ T cells. X-Axis: conditions. Y-Axis: % T-bet CD8+ T cells.

FIGS. 31A-31B show graphs of interferon expression and CD4+ T cell number/tumor volume in the tumor infiltrate. FIG. 31A shows interferon-gamma (IFNγ) expression in untreated and SEQ ID NO: 2/NP-1 treated tumors. X-Axis: conditions. Y-Axis: % IFN(CD4+ T cells. FIG. 31B shows intratumoral CD4+ cell number as a function of tumor volume. X-axis: conditions. Y-Axis: cell number/tumor volume.

FIG. 32 shows a graph of tumor volume over time after B16 inoculation in C57BL/6 mice. Conditions include diluent, SEQ ID NO: 2+NP-1 (1 dose); SEQ ID NO: 2 alone (1 dose); xRNA+NP-1 (1 dose); SEQ ID NO: 2 alone (3 doses); and SEQ ID NO: 2+NP-1 (3 doses). X-axis: days after B16 inoculation. Y-axis: tumor volume. Results are presented as mean for each group, all n=10. Results are presented though experimental day 14, after which removal of animals with tumors greater than the predetermined threshold of 1500 mm³ began to impact group-based calculations.

FIGS. 33A-33B show graphs of cell activation kinetics for controls, SEQ ID NO: 2 (alone) treated, and SEQ ID NO: 2+NP-1 treated C57BL/6 mice when administered via footpad injection. FIG. 33A shows a graph of the percentage of MoDC lymphocytes in the draining lymph node. FIG. 33B shows a graph of CCR7 expression in cDC1s in the draining lymphnode. X-axis: conditions. Y-axis: % of cells.

FIGS. 34A-34D show graphs of cell activation kinetics in the lung for controls, SEQ ID NO: 2 (alone) treated, and SEQ ID NO: 2+NP-1 treated C57BL/6 mice when administered intranasally. FIG. 34A shows a graph of CXCL10 expression in the lung over time for each condition. X-axis: condition. Y-axis: fold induction/control. FIG. 34B shows a graph of IFIT1 expression in the lung over time for each condition. X-axis: condition. Y-axis: fold induction/control. FIG. 34C shows a graph of IFIT2 expression in the lung over time for each condition. X-axis: condition. Y-axis: fold induction/control. FIG. 34D shows a graph of IFN-beta (IFNβ) expression in the lung over time for each condition. X-axis: condition. Y-axis: fold induction/control.

FIGS. 35A-35D show graphs of cell activation kinetics in the nasal cavity for controls, SEQ ID NO: 2 (alone) treated, and SEQ ID NO: 2+NP-1 treated C57BL/6 mice when administered intranasally. FIG. 35A shows a graph of CXCL10 expression in the nasal cavity over time for each condition. X-axis: condition. Y-axis: fold induction/control. FIG. 35B shows a graph of IFIT1 expression in the nasal cavity over time for each condition. X-axis: condition. Y-axis: fold induction/control. FIG. 35C shows a graph of IFIT2 expression in the nasal cavity over time for each condition. X-axis: condition. Y-axis: fold induction/control. FIG. 35D shows a graph of IFN-beta (IFNβ) expression in the nasal cavity over time for each condition. X-axis: condition. Y-axis: fold induction/control.

FIGS. 36A-36B show graphs of A549-Dual cells treated with SEQ ID NO: 2 complexed to various nanoparticles. FIG. 36A shows graphs of cell supernatants monitored for IFN-β by the presence of SEAP in the supernatant. FIG. 36B shows graphs of cell supernatants monitored for IFIT2 activation by the presence of luciferase in the supernatant.

FIGS. 37A-37B show graphs of A549-Dual cells were treated with SEQ ID NO: 2 complexed to various nanoparticles with 5 nm iron (Fe) particles, 15 nm Fe particles, or without Fe particles. FIG. 37A shows graphs of cell supernatants monitored for IFN-β by the presence of SEAP in the supernatant. FIG. 37B shows graphs of cell supernatants monitored for IFIT2 activation by the presence of luciferase in the supernatant.

FIGS. 38A-38B show graphs of A549-Dual cells were treated with SEQ TD NO: 2 complexed to various nanoparticles with various solid lipid cores or SLNs. FIG. 38A shows graphs of cell supernatants monitored for IFN-β by the presence of SEAP in the supernatant. FIG. 38B shows graphs of cell supernatants monitored for IFIT2 activation by the presence of luciferase in the supernatant. Solid lipid nanoparticles (SLNs) are compared to a standard (squalene-containing) NP-1 formulation.

FIG. 39 shows a graph of serum interferon levels in 7-11 week old C57BL/6 mice 14 hours after IM injection of PAMP complexed with 3 different lipid nanoparticles (LNPs).

### DETAILED DESCRIPTION

Provided herein are compositions, kits, devices and uses thereof for treatment of various conditions. Briefly, further provided herein are (1) pattern recognition receptor (PRR) agonists; (2) nanoparticle carriers systems; (3) combination compositions; (4) thermally stable, dried, and lyophilized compositions; (5) pharmaceutical compositions; (6) dosing; (7) administration; (8) methods and conditions.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

Unless specifically stated or apparent from context, as used herein, the term “about” in reference to a number or range of numbers is understood to mean the stated number and numbers +/−20% thereof, or 20% below the lower listed limit and 20% above the higher listed limit for the values listed for a range.

### Pattern Recognition Receptor (PRR) Agonists

Provided herein are nucleic acid sensor engaging compositions referred to as pattern recognition receptor (PRR) agonists. In some embodiments, the PRR agonists are a nucleic acid. The nucleic acid may be single-stranded or double-stranded. The nucleic acid may be RNA or DNA. The nucleic acid may be linear or include a hairpin. In some embodiments, the PRR is an endosomal nucleic acid sensor. In some embodiments, the endosomal nucleic acid sensor is toll-like receptor (TLR). Exemplary TLR PRRs include TLR3, TLR7, TLR8, and TLR9. In some embodiments, the TLR PRR is TLR3. In some embodiments, the TLR3 agonist is RIBOXXOL, poly(I:C), or Hiltonol®. In some embodiments, the PRR is a DNA sensor. Exemplary DNA sensor PRRs include cyclic GMP-AMP synthase (cGAS). In some embodiments, the PRR is a retinoic acid-inducible gene I (RIG-I)-like receptor (RLR). In some embodiments, the RLR is RIG-I, melanoma differentiation-associated protein 5 (MDA5), or laboratory of genetics physiology 2 (LGP2). In some embodiments, the PRR agonist is a viral RNA sequence, or a functional variant thereof. In some embodiments, the PRR agonist comprises a triphosphate (PPP) group at the 5′ end. In some embodiments, the PRR agonist comprising a triphosphate (PPP) group at the 5′ end is an RNA molecule. In some embodiments, the PRR agonist comprises an uncapped diphosphate (PP) group at the 5′ end. In some embodiments, the PRR agonist comprises an uncapped diphosphate (PP) group at the 5′ end is an RNA molecule. In some embodiments, the PRR agonist comprises a 5′-terminal nucleotide having an unmethylated 2′-0 position. In some embodiments, the PRR agonist binds to a carboxy-terminal domain (CTD) of an RLR. In some embodiments, the PRR agonist comprises nucleic acid base pairs which contact the helicase domain of an RLR. In some embodiments, the PRR agonist is an RLR agonist. In some embodiments, the RLR agonist is a RIG-I agonist. In some embodiments, the RIG-I agonist comprises a uridine rich stretch. In some embodiments, the RIG-I agonist comprises hepatitis C virus (HVC) RNA genome sequence, or a functional variant thereof. In some embodiments, the RIG-I agonist comprises Sendai virus RNA genome sequence, or a functional variant thereof. In some embodiments, the RIG-I agonist comprises any RNA genome sequence, or a functional variant thereof. In some embodiments, a composition herein includes a plurality of PRR agonist. In further embodiments, the plurality of PRR agonists have different sequences. In further embodiments, the plurality of PRR agonists comprise different RNA sequences. In further embodiments, the plurality of PRR agonists comprise different DNA sequences. In further embodiments, the plurality of PRR agonists comprise RNA and DNA sequences. In some embodiments, the PRR agonist comprises a nucleic acid coding a sequence listed in Table 1. In some embodiments, the PRR agonist comprises two or more nucleic acids coding different sequence listed in Table 1. In some embodiments, the PRR agonist is a nucleic acid comprising at least 80%, 85%, 90%, 95%, 96%, 97%, 98%, or 99% sequence identity to a sequence listed in Table 1. In some embodiments, the PRR agonist comprises two or more nucleic acids coding different sequence listed in Table 1. In some embodiments, the PRR agonist is a nucleic acid comprising at least 80%, 85%, 90%, 95%, 96%, 97%, 98%, or 99% sequence similarity to a sequence listed in Table 1. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

TABLE 1
PRR Agonists

| SEQ ID | |
|---|---|
| NO: | SEQUENCES |
| 1 | RNA 5′ |
| | CCAUCCUGUUUUUUUCCCUUUUUUUUUUUCUUUUUUUUUUUUUUUUU |
| | UUUUUUUUUUUUUUUUUCUCCUUUUUUUUUCCUCUUUUUUUCCUUUU |
| | CUUUCCUUU 3′ |
| 2 | RNA 5′ |
| | GGCCAUCCUGUUUUUUUCCCUUUUUUUUUUUCUUUUUUUUUUUUUUU |
| | UUUUUUUUUUUUUUUUUUUCUCCUUUUUUUUUCCUCUUUUUUUCCUU |
| | UUCUUUCCUUUUCUA 3′ |
| 3 | RNA 5′ CCCCCCCCCCCCCCCCCCCCCCCCCCCCCCCCCCCCCCCC |
| | CCCCCCCCCCCCCCCCCCCC 3′ |
| 4 | RNA 5′ |
| | GGGGGGGGGGGGGGGGGGGGGGGGGGGGGGGGGGGGGGGGGGGGGG |
| | GGGGGGGGGGGGGGGGGGGGGGGGGGGGGGGGGGGGGGGGGGGGGG |
| | GGGGGGGG 3′ |
| 5 | RNA 5′ TGCTGCTGCT TGCAAGCAGC TTGATACCAG ACAAAGCUGG |
| | GAAUAGAAAC UUCGUAUUUU CAAAGUUUUC UUUAAUAUAU |
| | UGCAAAUAAU GCCUAACCAC CUAGGGCAGG AUUAGGGUUC |
| | CGGAGUUCAA CCAAUUAGUC CUUAAUCAGG GCACUGUAUC CGACU |
| | 3′ |
| 6 | RNA 5′ AGUCGGAUAC AGUGCCCUGA UUAAGGACUA AUUGGUUGAA |
| | CUCCGGAACC CUAAUCCUGC CCUAGGUGGU UAGGCAUUAU |
| | UUGCAAUAUA UUAAAGAAAA CUUUGAAAAU ACGAAGUUUC |
| | UAUUCCCAGC UUUGUCUGGU 3′ |
| 7 | RNA 5′ pppGGAUCGAUCGAUCGUUCGCGAUCGAUCGAUCC-3′ |
| 8 | RNA 5′ GACGAAGACC ACAAAACCAG AUAAAAAAAA AAAAAAAAAA |
| | AAAAAAAAUA AUUUUUUUUU UUUUUUUUUU UUUUUUUAUC |
| | UGGUUUUGUG GUCUUCGUC 3′ |
| 9 | RNA 5′ ppp-GGACGUACGUUUCGACGUACGUCC 3′ |
| 10 | RNA 5′- |
| | pppAGCAAAAGCAGGGUGACAAAGACAUAAUGGAUCCAAACACUGUGU |
| | CAAGCUUUCAGGUAGAUUGCUUUCUUUGGCAUGUCCGCAAAC-3′ |
| 11 | DNA |
| | 5′pppTAATACGACTCACTATAGGCCATCCTGTTTTTTTCCCTTTTTTTTTT |
| | CTTTTTTTTTTTTTTTTTTTTTTTTTTTTTTTTTTTCTCCTTTTTTTTTCCTCT |
| | TTTTTTCCTTTTCTTTCCTTT-3 |

In some embodiments, nucleic acid PRR agonists disclosed herein may be present in a composition provided herein and are present in nanogram or microgram amounts. Exemplary amounts for PRR agonists disclosed herein include about 0.05, 0.1, 0.2, 0.3, 0.4, 0.5, 0.6, 0.7, 0.8, 0.9, 1, 1.2, 1.25, 1.5, 2, 3, 4, 5, 7.5, 10 or more μg. Exemplary amounts for PRR agonists disclosed herein include up to 0.1, 0.2, 0.3, 0.4, 0.5, 0.6, 0.7, 0.8, 0.9, 1, 1.2, 1.25, 1.5, 2, 3, 4, 5, 7.5, or 10 μg. Exemplary amounts for PRR agonists disclosed herein include at least 0.05, 0.1, 0.2, 0.3, 0.4, 0.5, 0.6, 0.7, 0.8, 0.9, 1, 1.2, 1.25, 1.5, 2, 3, 4, 5, 7.5, or 10 μg. Exemplary amounts for PRR agonists disclosed herein include 0.05 to 10, 0.1 to 5, 0.05 to 5, 0.1 to 5 μg. Additional exemplary amounts for PRR agonists disclosed herein include about 0.05, 0.1, 0.2, 0.5, 1, 5, 10, 12.5, 15, 25, 40, 50, 100, 125, 150, 175, 200, 250, 400, 500, 600, 700, 750, 1000, 1500, 2000, 3000, 4000, 5000 or more ug. In some embodiments, the nucleic acid PRR agonist comprises one or more nucleic acids comprising a sequence disclosed in Table 1. In some embodiments, the nucleic acid is at least about 15, 25, 50, 75, 100, 125, 150, 175, 200, 225, 250, 275, or 300 nucleotides in length. In some embodiments, the nucleic acid is up to about 15, 25, 50, 75, 100, 125, 150, 175, 200, 225, 250, 275, or 300 nucleotides in length. In some embodiments, the nucleic acid is 25-150, 25-300, or 50-150 nucleotides in length.

In some embodiments, the PRR agonist is a nucleic acid, such as an RNA or DNA. A variety of RNAs can be associated with the nanoparticles for delivery provided herein, including RNAs that modulate innate immune responses, RNAs that encode proteins or antigens, silencing RNAs, microRNAs, tRNAs, self-replicating RNAs, etc. In certain embodiments, the PRR agonist is a non-coding RNA, a TLR agonist, a RIG-I agonist, a saponin, a peptide, a protein, a carbohydrate, a carbohydrate polymer, a conjugated carbohydrate, a whole viral particle, a virus-like particle, viral fragments, cellular fragments, and combinations thereof. In certain embodiments, the nucleic acid is a TLR agonist or a RIG-I agonist. Exemplary TLR agonists include a TLR2, TLR3, TLR4, TLR7, TLR8, or TLR9 agonist. An exemplary TLR agonist for inclusion in a composition provided herein is, without limitation, is a TLR3 agonist, such as RIBOXXOL, poly(I.C) (Polyinosinic:polycytidylic acid, sodium salt, ((C1OH10N4NaO7P)x·(C9H11N3NaO7P)x)), or Hiltonol®.

### Nanoparticles

Provided herein are various compositions comprising a nanoparticle. In some embodiments, the nanoparticle comprises a lipid carrier. Nanoparticles are abbreviated as NPs herein. Nanoparticles provided herein may be an organic, inorganic, or a combination of inorganic and organic materials that are less than about 1 micrometer (μm) in diameter. In some embodiments, nanoparticles provided herein are used as a delivery system for a bioactive agent provided herein (e.g., a nucleic acid comprising or encoding for a PRR). Further provided herein are various compositions comprising lipid carrier complexes or nanoparticle-complexes, wherein a plurality of lipid carriers or a plurality of nanoparticles interact physically, chemically, and/or covalently. The specific type of interaction between lipid carriers or between nanoparticles will depend upon the characteristic shapes, sizes, chemical compositions, physical properties, and physiologic properties. Nanoparticles provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

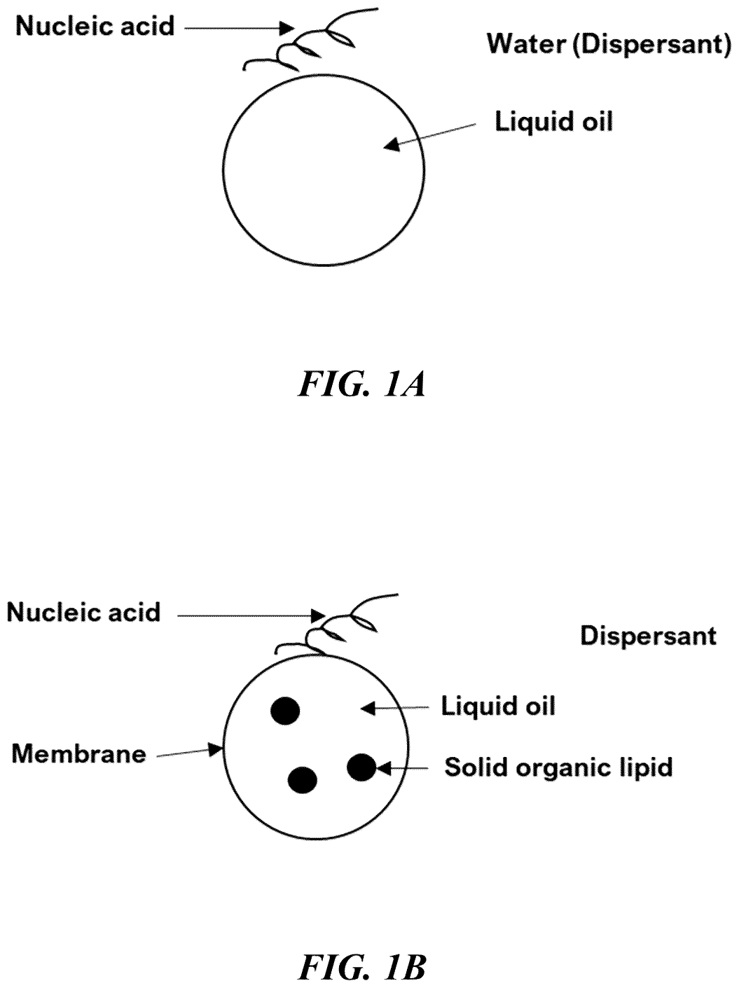

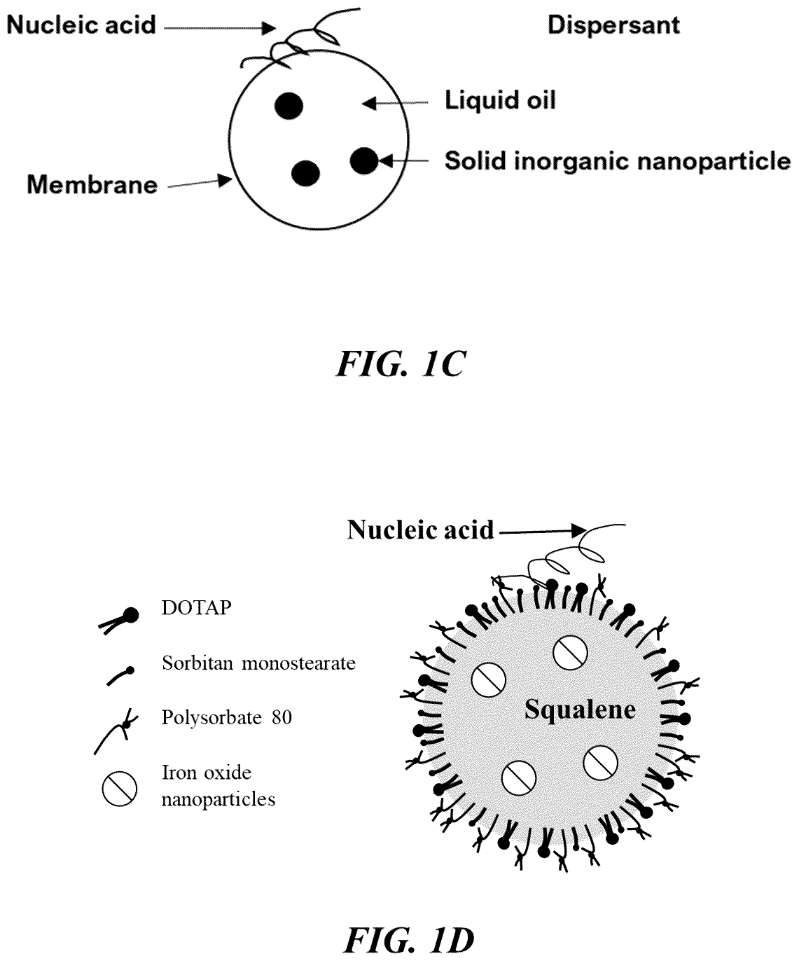

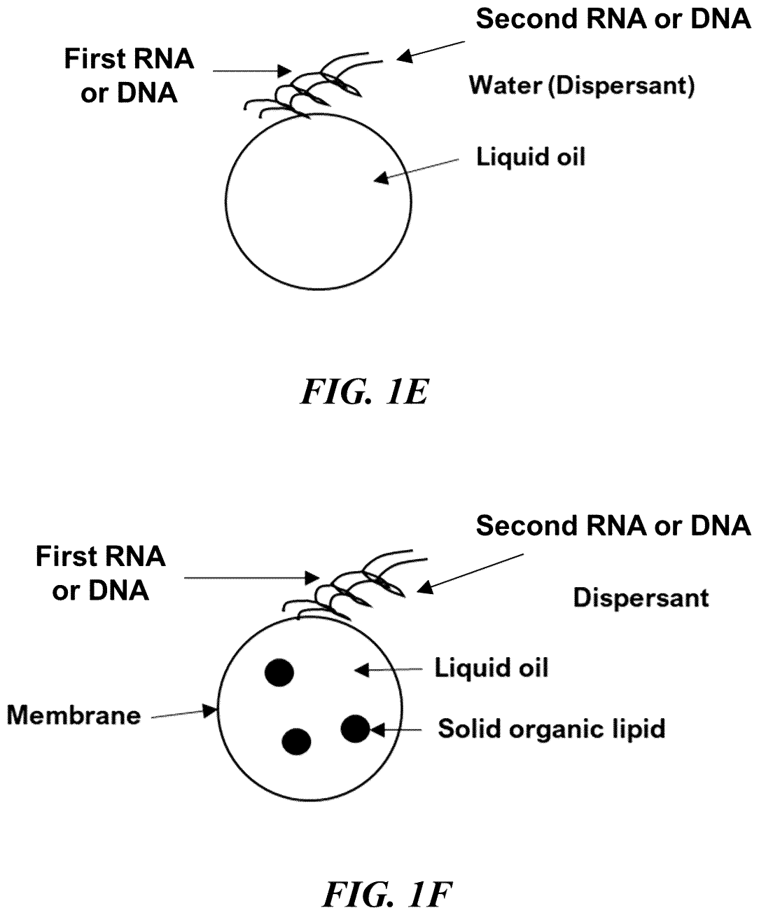

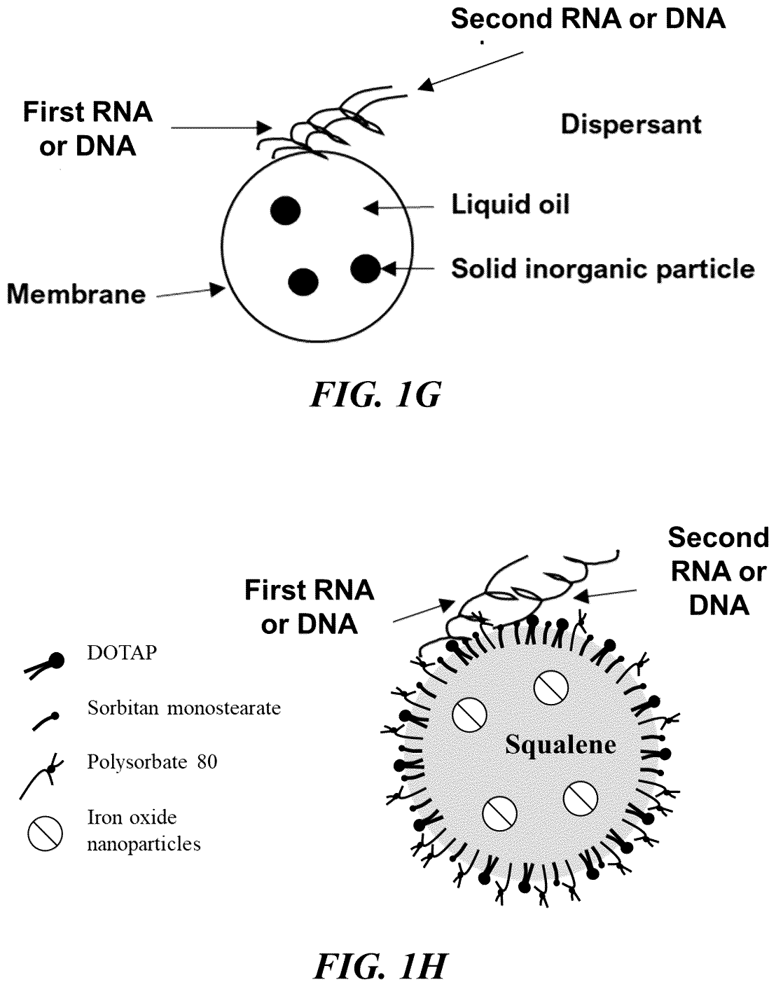

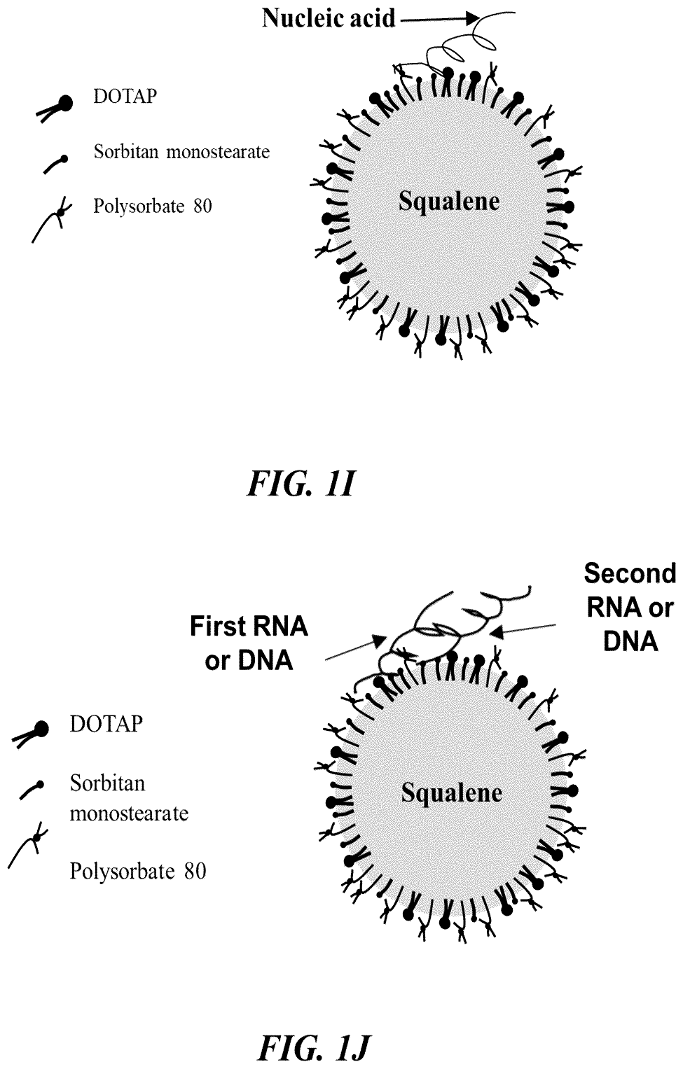

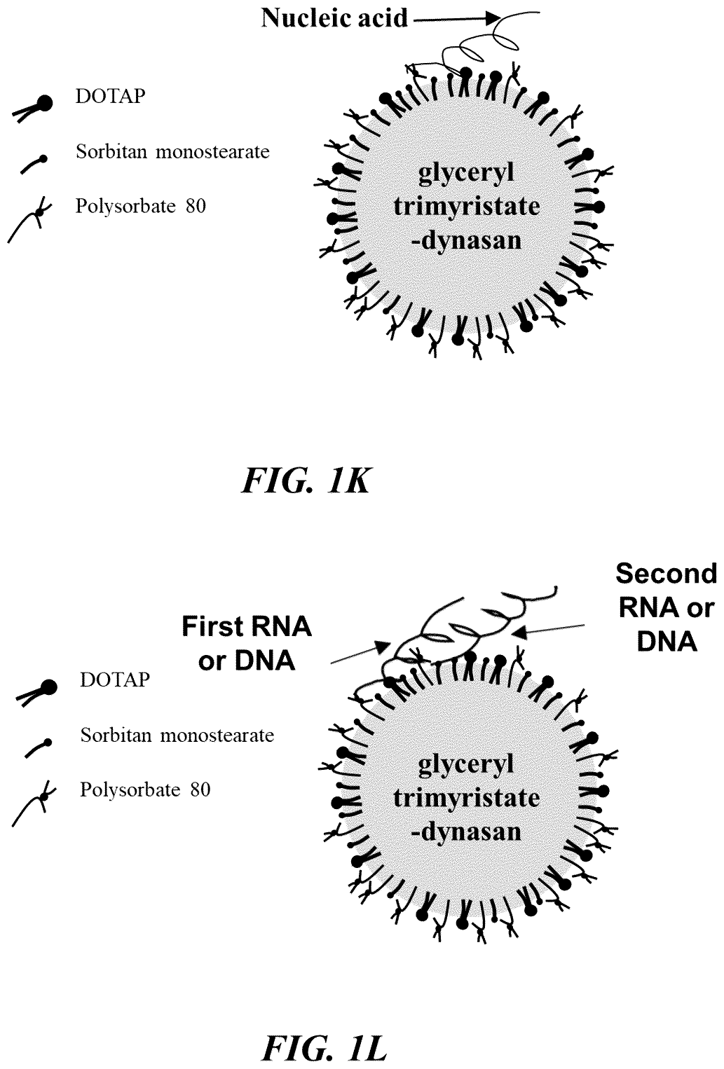

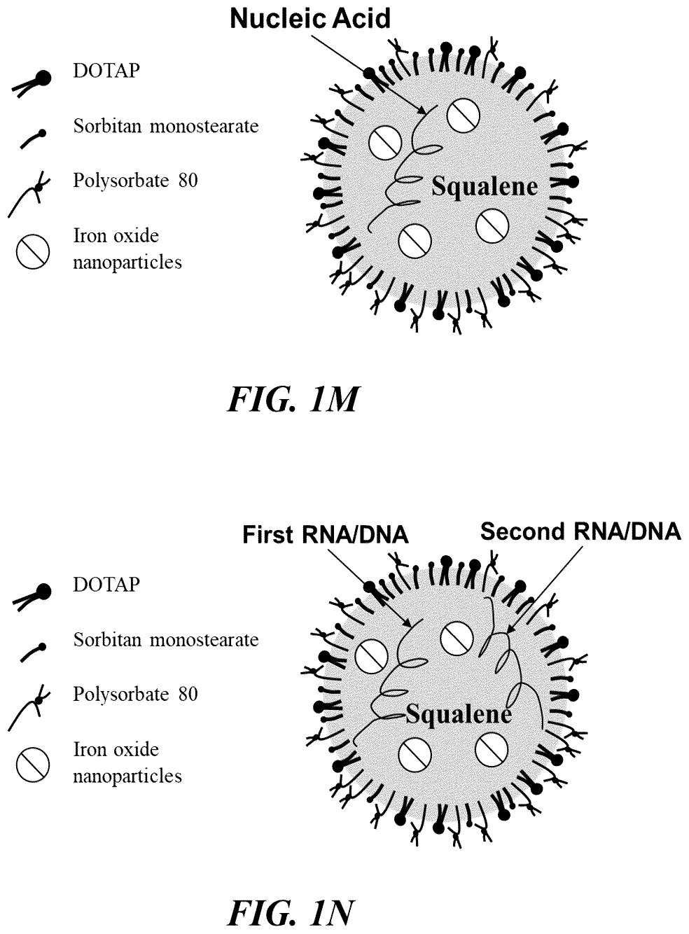

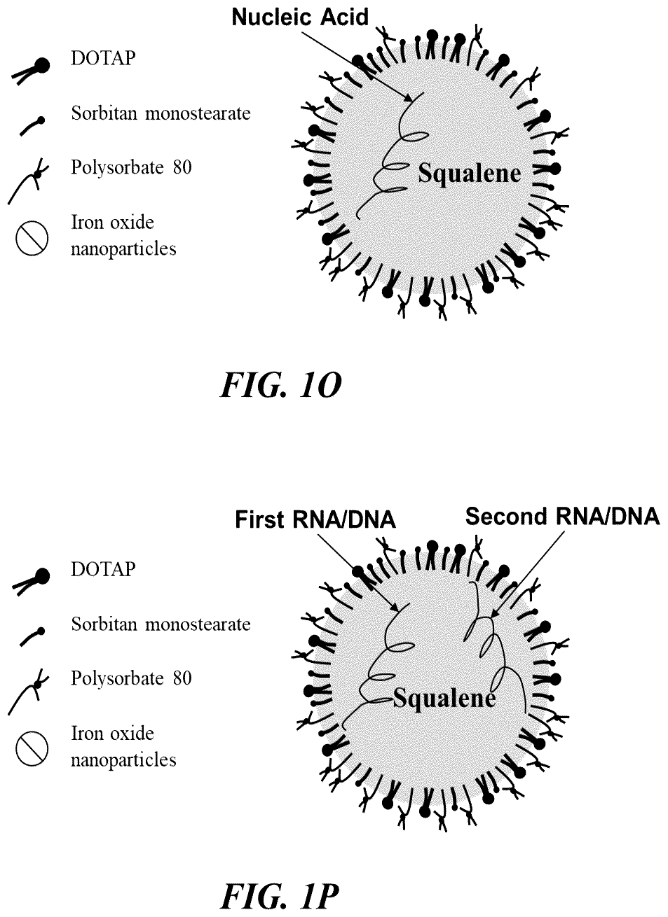

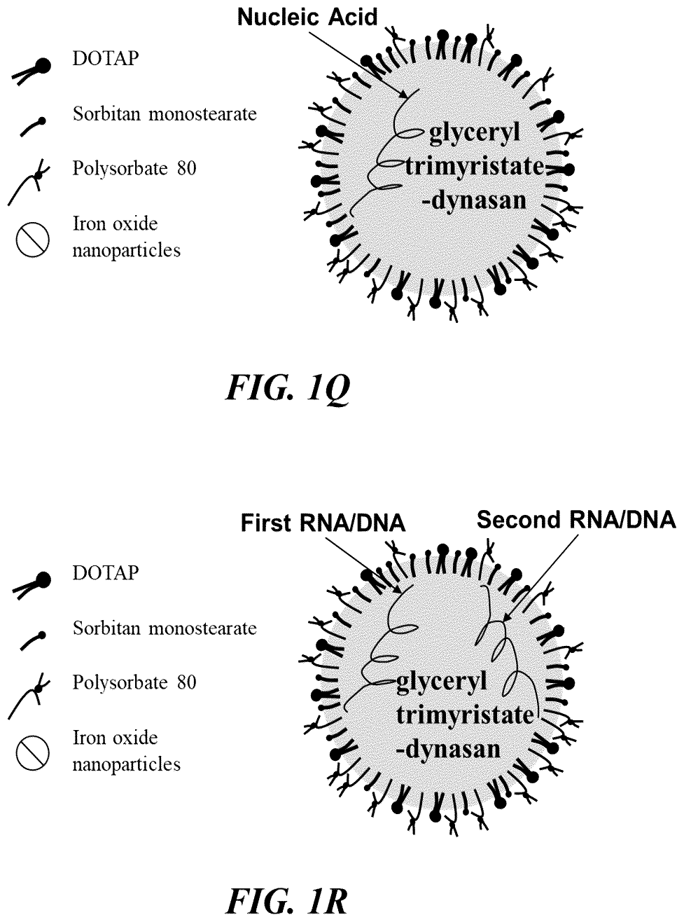

Various nanoparticles and formulations of nanoparticles (i.e., nanoemulsions) are employed. Exemplary nanoparticles are illustrated in FIGS. 1A-1L. Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. FIG. 1D (not to scale) illustrates a nanoparticle comprising a cationic lipid membrane and a liquid oil without an inorganic particle. FIGS. 1I-1J illustrate a nanoparticle comprising a cationic lipid membrane (e.g., DOTAP) and a liquid oil core comprising squalene without an inorganic particle. In some embodiments, a nanoparticle provided herein comprises a solid core comprising glyceryl trimyristate-dynasan (FIGS. 1K-1L).

Nucleic acids provided herein can be complexed with a nanoparticle in Table 2 in cis (FIGS. 1A-1D) or in trans (FIGS. 1E-1L). For example, a first RNA or DNA molecule can comprise a first PRR sequence and an additional PRR sequence on the same nucleic acid. As another example, a first RNA or DNA molecule can comprise a first PRR sequence; and a second RNA or DNA molecule can comprise an additional PRR. A nucleic acid provided herein can optionally comprise an RNA polymerase or an RNA polymerase complex. In some embodiments, the RNA polymerase or RNA polymerase complex comprises a Venezuelan equine encephalitis virus (VEEV) RNA polymerase.

Provided herein are nanoemulsions and nanodroplets comprising a plurality of lipid carriers or nanoparticles, wherein each lipid carrier or nanoparticle comprises a cationic lipid. In some embodiments, nanoemulsions comprises a plurality of cationic lipid carriers. In some embodiments, a composition provided herein comprises a cationic nanoemulsion. In some embodiments, cationic nanoemulsions provided herein comprise lipid (or other surfactant) molecules surrounding an oil particle that is dispersed in water and give the oil particle a cationic (positively charged) surface to which negatively-charged RNA molecules can adhere.

The entire nanodroplet can be dispersed as a colloid in the aqueous (water) phase or in a suspension. In some embodiments, nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxy-dodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticle provided herein comprises a hydrophobic lipid core. In some embodiments, the hydrophobic lipid core is in liquid phase at 25 degrees C. Non-limiting examples of hydrophobic lipid core components that can be used include α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins. In some embodiments, the hydrophobic lipid is in solid phase. In some embodiments, the hydrophobic lipid is in liquid phase, also referred to as an oil. In some embodiments, the hydrophobic lipid comprises squalene. In some embodiments, the hydrophobic lipid comprises solanesol.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. In some embodiments, the nanoparticle provided herein comprises the inorganic particle within the hydrophobic core. In some embodiments, the nanoparticle provided herein comprises a metal. In some embodiments, the nanoparticle provided herein comprises a metal within the hydrophobic core. The metal can be without limitation, a metal salt such as a transition metal salt, a metal oxide such as a transition metal oxide, a metal hydroxide such as a transition metal hydroxide, a metal phosphate such as a transition metal phosphate, or a metalloid (e.g., silicon and silicon-based compounds or alloys). In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal). In some embodiments, the inorganic particle is a transition metal oxide. In some embodiments, the transition metal is magnetite (Fe₃O₄), maghemite (y—Fe₂O₃), wüstite (FeO), or hematite (alpha (α)— Fe₂O₃). In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, nanoparticles provided herein comprise a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant.

In some embodiments, nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85).

Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5. In some embodiments, nanoparticles provided herein comprise a ratio of the esters that yields a hydrophilic-lipophilic balance between 8 and 11. HLB is used to categorize surfactants as hydrophilic or lipophilic. The HLB scale provides for the classification of surfactant function calculated e.g., by Griffin's method:

$$HLB = \frac{20 {M}_{h}}{M} ,$$

where Mₕ is the molecular mass of the hydrophilic portion of the lipid carrier and M is the molecular mass of the lipid carrier. The HLB scale is provided below:
HLB=0: fully lipophilic/hydrophobic carrier;
HLB between 0 and 6 is an oil soluble carrier;
HLB between 6 and 9 is a water dispersible carrier;
HLB between 9 and 20 is a hydrophilic, water soluble carrier;
HLB=20: fully hydrophilic/lipophobic carrier.

In some embodiments, a nanoparticle or a lipid carrier provided herein comprises a hydrophilic surfactant, also called an emulsifier. In some embodiments, a nanoparticle or a lipid carrier provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as TWEEN®, Kolliphor, Scattics, Alkest, or Canarcel; polyoxyethylene sorbitan ester (polysorbate); polysorbate 80 (polyoxyethylene sorbitan monooleate, or TWEEN® 80); polysorbate 60 (polyoxyethylene sorbitan monostearate, or TWEEN® 60); polysorbate 40 (polyoxyethylene sorbitan monopalmitate, or TWEEN® 40); and polysorbate 20 (polyoxyethylene sorbitan monolaurate, or TWEEN® 20). In one embodiment, the hydrophilic surfactant is polysorbate 80.

In some embodiments, nanoparticles and lipid carriers provided herein comprise a hydrophobic core surrounded by a lipid membrane (e.g., a cationic lipid such as DOTAP). In some embodiments, the hydrophobic core comprises: one or more inorganic particles; a phosphate-terminated lipid; and a surfactant.

Inorganic solid nanoparticles provided herein can be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises a surfactant such as a phosphorous-terminated surfactant, a carboxylate-terminated surfactant, a sulfate-terminated surfactant, or an amine-terminated surfactant. Exemplary carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 2% to about 6% w/v squalene, from about 0.01% to about 1% w/v iron oxide nanoparticles, from about 0.2% to about 1% w/v DOTAP, from about 0.25 to about 1% w/v sorbitan monostearate, and from about 0.50% to about 5% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTP, from about 0.25% to about 1% y w/v sorbitan monostearate, and from about 0.50) to about 50 w/v polysorbate 80.

In some embodiments, a composition provided herein comprises at least one nanoparticle formulation as described in Table 2. In some embodiments, a composition provided herein comprises any one of NP-1 to NP-31. In some embodiments, a composition provided herein comprises any one of NP-1 to NP-37. In some embodiments, the nanoparticles provided herein are admixed with a nucleic acid provided herein. In some embodiments, nanoparticles provided herein are made by homogenization and ultrasonication techniques.

TABLE 2
Nanoparticle Formulations.

| | Cationic Lipid (s) % | Oil (s) | Surfactant (s) | Additional Ingredients |
|---|---|---|---|---|
| Name | (w/v) or mg/ml | % (w/v) or mg/ml | % (w/v) or mg/ml | % (w/v), mg/ml, or mM |
| NP-1 | 30 mg/ml 1,2-dioleoyl-3- | 37.5 mg/ml squalene | 37 mg/ml sorbitan | 0.2 mg Fe/ml 12 nm oleic |
| | trimethylammonium-propane | | monostearate, (2R)- | acid-coated iron oxide |
| | (DOTAP) chloride | | 2-[(2R,3R,4S)-3,4- | nanoparticles |
| | | | Dihydroxyoxolan-2- | 10 mM sodium citrate |
| | | | yl]-2-hydroxyethyl | dihydrate. |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-2 | 30 mg/ml 1,2- dioleoyl-3- | 37.5 mg/ml squalene | 37 mg/ml sorbitan | 1 mg Fe/ml 15 nm oleic |
| | trimethylammonium- propane | | monostearate (2R)- | acid-coated iron oxide |
| | (DOTAP) chloride | | 2-[(2R,3R,4S)-3,4- | nanoparticles |
| | | | Dihydroxyoxolan-2- | 10 mM sodium citrate |
| | | | yl]-2-hydroxyethyl | dihydrate |
| | | | octadecenoate | |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-3 | 30 mg/ml 1,2- dioleoyl-3- | 37.5 mg/ml Miglyol | 37 mg/ml sorbitan | 0.2 mg Fe/ml 15 nm oleic |
| | trimethylammonium-propane | 812 N | monostearate, (2R)- | acid-coated iron oxide |
| | (DOTAP) chloride | (triglyceride ester of | 2-[(2R,3R,4S)-3,4- | nanoparticles |
| | | saturated | Dihydroxyoxolan-2- | 10 mM sodium citrate |
| | | coconut/palmkernel | yl]-2-hydroxyethyl | dihydrate |
| | | oil derived caprylic | octadecenoate | |
| | | and capric fatty acids | C₂₄H₄₆O₆ | |
| | | and plant derived | (SPAN ® 60) | |
| | | glycerol) | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-4 | 30 mg/ml 1,2-dioleoyl-3- | 37.5 mg/ml Miglyol | 37 mg/ml sorbitan | 1 mg Fe/ml 15 nm oleic |
| | trimethylammonium- propane | 812 N | monostearate, (2R)- | acid-coated iron oxide |
| | (DOTAP) chloride | (triglyceride ester of | 2-[(2R,3R,4S)-3,4- | nanoparticles |
| | | saturated | Dihydroxyoxolan-2- | 10 mM sodium citrate |
| | | coconut/palmkernel | yl]-2-hydroxyethyl | dihydrate. |
| | | oil derived caprylic | octadecenoate, | |
| | | and capric fatty acids | C₂₄H₄₆O₆) | |
| | | and plant derived | (SPAN ® 60) | |
| | | glycerol) | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-5 | 30 mg/ml DOTAP chloride | 37.5 mg/ml squalene | 37 mg/ml sorbitan | 1 mg/ml trioctylphosphine |
| | | | monostearate | oxide (TOPO)-coated |
| | | | (SPAN ® 60) | aluminum hydroxide |
| | | | 37 mg/ml | (Alhydrogel ® 2%) |
| | | | polysorbate 80 | particles |
| | | | (TWEEN ® 80) | 10 mM sodium citrate |
| | | | | dihydrate |
| NP-6 | 30 mg/ml DOTAP chloride | 37.5 mg/ml Solaneso | 37 mg/ml sorbitan | 0.2 mg Fe/ml oleic acid- |
| | | (Cayman chemicals) | monostearate | coated iron oxide |
| | | | (SPAN ® 60) | nanoparticles |
| | | | 37 mg/ml | 10 mM sodium citrate |
| | | | polysorbate 80 | |
| | | | (TWEEN ® 80) | |
| NP-7 | 30 mg/ml DOTAP chloride | 37.5 mg/ml squalene | 37 mg/ml sorbitan | 10 mM sodium citrate |
| | | 2.4 mg/ml glyceryl | monostearate | |
| | | trimyristate-dynasan | (SPAN ® 60) | |
| | | (DYNASAN 114 ®) | 37 mg/ml | |
| | | | polysorbate 80 | |
| | | | (TWEEN ® 80) | |
| NP-8 | 4 mg/ml DOTAP chloride | 43 mg/ml squalene | 5 mg/ml sorbitan | 10 mM sodium citrate |
| | | | trioleate | |
| | | | (SPAN ® 85) | |
| | | | 5 mg/ml polysorbate | |
| | | | 80 (TWEEN ® 80) | |
| NP-9 | 7.5 mg/ml 1,2-dioleoyl-3- | 9.4 mg/ml squalene | 9.3 mg/ml sorbitan | 0.05 mg/ml 15 nanometer |
| | trimethy lammonium-propane | ((6E,10E,14E,18E)- | monostearate (2R)- | superparamagnetic iron |
| | (DOTAP) chloride | 2,6,10,15,19,23- | 2-[(2R,3R,4S)-3,4- | oxide (Fe₃O₄) |
| | | Hexamethyltetracosa- | Dihydroxyoxolan-2- | 10 mM sodium citrate |
| | | 2,6,10,14,18,22- | yl]-2-hydroxyethyl | |
| | | hexaene, C₃₀H₅₀) | octadecenoate, | dihydrate |
| | | 0.63 mg/ml glyceryl | C₂₄H₄₆O₆) | |
| | | trimyristate-dynasan | (SPAN ® 60) | |
| | | (DYNASAN 114 ®) | 9.3 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-10 | 0.4% DOTAP | 0.25% glyceryl | 0.5% sorbitan | |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-11 | 3.0% DOTAP | 0.25% glyceryl | 3.7% sorbitan | |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-12 | 0.4% DOTAP | 4.3% Squalene | 0.5% sorbitan | |
| | | | trioleate | |
| | | | (SPAN ® 85) | |
| | | | 0.5% polysorbate 80 | |
| | | | (TWEEN ® 80) | |
| NP-13 | 0.4% DOTAP | 0.25% glyceryl | 2.0% polysorbate 80 | |
| | | trimyristate-dynasan | (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80) | |
| NP-15 | 0.4% DOTAP | 0.25% glyceryl | 0.25% sorbitan | |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80) | |
| NP-16 | 0.4% DOTAP | 5% squalene | 0.5% sorbitan | |
| | | | trioleate | |
| | | | (SPAN ® 85) | |
| | | | 2.0% polysorbate 80 | |
| | | | (TWEEN ® 80) | |
| NP-17 | 0.4% DOTAP | 5% squalene | 0.5% sorbitan | |
| | | | monostearate | |
| | | | (SPAN ® 60) | |
| | | | 2% polysorbate 80 | |
| | | | (TWEEN ® 80) | |
| NP-18 | 0.4% DOTAP | 0.25% glyceryl | 2% sorbitan trioleate | |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-19 | 0.4% DOTAP | 0.25% glyceryl | 0.5% sorbitan | 1% aluminum hydroxide |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-20 | 3.0% DOTAP | 0.25% glyceryl | 3.7% sorbitan | 1% aluminum hydroxide |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-21 | 0.4% DOTAP | 4.3% Squalene | 0.5% sorbitan | 1% aluminum hydroxide |
| | | | trioleate | |
| | | | (SPAN ® 85) | |
| | | | 0.5% polysorbate 80 | |
| | | | (TWEEN ® 80 | |
| NP-22 | 0.4% DOTAP | 0.25% glyceryl | 2.0% polysorbate 80 | 1% aluminum hydroxide |
| | | trimyristate-dynasan | (TWEEN ® 80) | |
| | | (DYNASAN 114 ®) | | |
| | | 4.08% squalene | | |
| NP-23 | 0.4% DOTAP | 0.25% glyceryl | 0.5% sorbitan | 1% aluminum hydroxide |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80 | |
| NP-24 | 0.4% DOTAP | 0.25% glyceryl | 0.25% sorbitan | 1% aluminum hydroxide |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80) | |
| NP-25 | 0.4% DOTAP | 5% squalene | 0.5% sorbitan | 1% aluminum hydroxide |
| | | | trioleate | |
| | | | (SPAN ® 85) | |
| | | | 2.0% polysorbate 80 | |
| | | | (TWEEN ® 80) | |
| NP-26 | 0.4% DOTAP | 5% squalene | 0.5% sorbitan | 1% aluminum hydroxide |
| | | | monostearate | |
| | | | (SPAN ® 60) | |
| | | | 2% polysorbate 80 | |
| | | | (TWEEN ® 80) | |
| NP-27 | 0.4% DOTAP | 0.25% glyceryl | 2% sorbitan trioleate | 1% aluminum hydroxide |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-28 | 0.5-5.0 mg/ml DOTAP | 0.2-10% (v/v) | 0.01-2.5% (v/v) | |
| | | squalene | polysorbate 80 | |
| | | | (TWEEN ® 80) | |
| NP-29 | 0.4% (w/w) DOTAP | 4.3% (w/w) squalene | 0.5% (w/w) sorbitan | |
| | | | trioleate | |
| | | | (SPAN ® 85) | |
| | | | 0.5% (w/w) | |
| | | | polysorbate 80 | |
| | | | (TWEEN ® 80) | |
| NP-30 | 30 mg/ml DOTAP chloride | 37.5 mg/ml squalene | 37 mg/ml sorbitan | 10 mM sodium citrate |
| | | | monostearate | |
| | | | (SPAN ® 60) | |
| | | | 37 mg/ml | |
| | | | polysorbate 80 | |
| | | | (TWEEN ® 80) | |
| NP-31 | 30 mg/ml DOTAP chloride | 37.5 mg/ml squalene | 37 mg/ml sorbitan | 0.4 mg Fe/ml 5 nm oleic |
| | | | monostearate | acid-coated iron oxide |
| | | | (SPAN ® 60) | nanoparticles |
| | | | 37 mg/ml | 10 mM sodium citrate |
| | | | polysorbate 80 | dihydrate |
| | | | (TWEEN ® 80) | |
| NP-32 | 0.8-1.6 mg/ml DOTAP | 4.5% squalene | 0.5% (w/w) sorbitan | 10 mM sodium citrate |
| | chloride | | trioleate | |
| | | | (SPAN 85 ®) | |
| | | | 0.5% (w/w) | |
| | | | polysorbate 80 | |
| | | | (TWEEN ® 80) | |
| NP-33 | 45-55 mol % ionizable | 35-42 mol % | 1.25-1.75 mol % | |
| | cationic lipid | cholesterol | PEG2000-DMG | |
| | 8-12 mol % | | | |
| | distearoylphosphatidylcholine | | | |
| | (DSPC) | | | |
| NP-34 | 50 mol % D-Lin-MC3-DMA | 38.5% cholesterol | 1.5% PEG-lipid | |
| | (MC3) | | | |
| | 10 mol % | | | |
| | distearoylphosphatidylcholine | | | |
| | (DSPC) | | | |
| NP-35 | 50 mol % Lipid H (SM-102) | 38.5% cholesterol | 1.5 mol % | |
| | 10 mol % | | PEG2000-DMG | |
| | distearoylphosphatidylcholine | | | |
| | (DSPC) | | | |
| NP-36 | 30 mg/ml 1,2-dioleoyl-3- | 3.75% w/v glyceryl | 37 mg/ml sorbitan | 10 mM sodium citrate |
| | trimethylammonium-propane | trimyristate-dynasan | monostearate, (2R)- | dihydrate. |
| | (DOTAP) chloride | (DYNASAN 114 ®) | 2-[(2R,3R,4S)-3,4- | |
| | | | Dihydroxyoxolan-2- | |
| | | | yl]-2-hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-37 | 30 mg/ml 1,2- dioleoyl-3- | 3.75% w/v glyceryl | 37 mg/ml sorbitan | 0.2 mgFe/mL or 0.02% |
| | trimethylammonium-propane | trimyristate-dynasan | monostearate, (2R)- | wFe/v of 5 to 15 nm |
| | (DOTAP) chloride | (DYNASAN 114 ®) | 2-[(2R,3R,4S)-3,4- | diameter iron oxide |
| | | | Dihydroxyoxolan-2- | nanoparticles |
| | | | yl]-2-hydroxyethyl | 10 mM sodium citrate |
| | | | octadecenoate, | dihydrate. |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |

In some embodiments, nanoparticles provided herein comprise: sorbitan monostearate (e.g., SPAN® 60), polysorbate 80 (e.g., TWEEN® 80), DOTAP, squalene, and no solid particles. In some embodiments, nanoparticles provided herein comprise: sorbitan monostearate (e.g., SPAN® 60), polysorbate 80 (e.g., TWEEN® 80), DOTAP, squalene, and iron oxide particles. In some embodiments, nanoparticles provided herein comprise an immune stimulant. In some embodiments, the immune stimulant is squalene. In some embodiments, the immune stimulant is Miglyol 810 or Miglyol 812. Miglyol 810 is a triglyceride ester of saturated caprylic and capric fatty acids and glycerol. Miglyol 812 is a triglyceride ester of saturated coconut/palmkernel oil derived caprylic and capric fatty acids and plant derived glycerol. In some embodiments, the immune stimulant can decrease the total amount of protein produced, but can increase the immune response to a composition provided herein. In some embodiments, the immune stimulant can increase the total amount of protein produced, but can decrease the immune response to a composition provided herein.

Nanoparticles provided herein can be of various average diameters in size. In some embodiments, nanoparticles provided herein are characterized as having an average diameter (z-average hydrodynamic diameter, measured by dynamic light scattering) ranging from about 20 nanometers (nm) to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle) ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, the inorganic particle (e.g., iron oxide) within the hydrophobic core of the nanoparticle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm. In some embodiments, the ratio of esters and lipids yield a particle size between 30 nm and 200 nm. In some embodiments, the ratio of esters and lipids yield a particle size between 40 nm and 70 nm.

Nanoparticles provided herein may be characterized by the polydispersity index (PDI), which is an indication of their quality with respect to size distribution. In some embodiments, average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

### Combination Compositions

Provided herein are compositions comprising a nanoparticle provided herein and a nucleic acid provided herein. In some embodiments, the nanoparticle comprises NP-1. In some embodiments, the nanoparticle comprises NP-2. In some embodiments, the nanoparticle comprises NP-3. In some embodiments, the nanoparticle comprises NP-4. In some embodiments, the nanoparticle comprises NP-5. In some embodiments, the nanoparticle comprises NP-6. In some embodiments, the nanoparticle comprises NP-7. In some embodiments, the nanoparticle comprises NP-8. In some embodiments, the nanoparticle comprises NP-9. In some embodiments, the nanoparticle comprises NP-10. In some embodiments, the nanoparticle comprises NP-11. In some embodiments, the nanoparticle comprises NP-12. In some embodiments, the nanoparticle comprises NP-13. In some embodiments, the nanoparticle comprises NP-14. In some embodiments, the nanoparticle comprises NP-15. In some embodiments, the nanoparticle comprises NP-16. In some embodiments, the nanoparticle comprises NP-17. In some embodiments, the nanoparticle comprises NP-18. In some embodiments, the nanoparticle comprises NP-18. In some embodiments, the nanoparticle comprises NP-19. In some embodiments, the nanoparticle comprises NP-20. In some embodiments, the nanoparticle comprises NP-21. In some embodiments, the nanoparticle comprises NP-22. In some embodiments, the nanoparticle comprises NP-23 In some embodiments, the nanoparticle comprises NP-24. In some embodiments, the nanoparticle comprises NP-25. In some embodiments, the nanoparticle comprises NP-26. In some embodiments, the nanoparticle comprises NP-27. In some embodiments, the nanoparticle comprises NP-28. In some embodiments, the nanoparticle comprises NP-28. In some embodiments, the nanoparticle comprises NP-29. In some embodiments, the nanoparticle comprises NP-30. In some embodiments, the nanoparticle comprises NP-31. In some embodiments, the nanoparticle comprises NP-32. In some embodiments, the nanoparticle comprises NP-33. In some embodiments, the nanoparticle comprises NP-34. In some embodiments, the nanoparticle comprises NP-34. In some embodiments, the lipid carrier comprises any of NP-1 to NP-31, or any one of NP-1 to NP-37; and a cryoprotectant. In some embodiments, the cryoprotectant is a sugar provided herein.

Further provided herein is a nanoemulsion comprising a plurality of nanoparticles provided herein and one or more nucleic acid. Nucleic acids for inclusion comprise a region that includes, without limitation, any one of, or a plurality of, SEQ ID NOS: 1-11.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to an antigen and increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1 In some embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In some embodiments, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene:cationic lipid, hydrophobic surfactant, and hydrophilic surfactant. In some embodiments, the oil-to-surfactant ratio is the molar ratio of squalene:DOTAP, hydrophobic surfactant, and hydrophilic surfactant. In some embodiments, the oil-to-surfactant ratio is the molar ratio of squalene:DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-cationic lipid ratio. In some embodiments, the hydrophilic surfactant-to-cationic lipid ratio ranges from about 0.1:1 to about 2:1, from about 0.2:1 to about 1.5:1,from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1. In some embodiments, the cationic lipid is DOTAP.

Provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN® 60), polysorbate 80 (e.g., TWEEN® 80), DOTAP, an immune stimulant, and an RNA.

### Thermally Stable, Dried, and Lyophilized Compositions

Provided herein are dried or lyophilized compositions comprising a nucleic acid provided herein. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

A lyophilized composition is generated by a low temperature dehydration process involving the freezing of the composition, followed by a lowering of pressure, and removal of ice by sublimation. In certain cases, lyophilization also involves the removal of bound water molecules through a desorption process. In some embodiments, compositions provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions provided herein at higher temperatures (e.g., greater than 4° C.) as compared to the sub-zero temperatures needed for the storage of existing mRNA therapeutics. In some embodiments, dried compositions and lyophilized compositions provided herein comprise (a) a lipid carrier, wherein the lipid carrier is a nanoemulsion comprising: (i) a hydrophobic core; (ii) one or more inorganic nanoparticles; (iii) and one or more lipids; (b) one or more nucleic acids; and (c) at least one cryoprotectant. In some embodiments, the cryoprotectant is selected from the group consisting of: sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), olyethylene glycol (PEG), 1,2-propanediol, acetamide, trehalose, formamide, sugars, proteins, and carbohydrates.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least about 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least about 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions provided herein are thermally stable. A composition is considered thermally stable when the composition resists the action of heat or cold and maintains its properties, such as the ability to protect a nucleic acid molecule from degradation at given temperature. In some embodiments, compositions provided herein are thermally stable at about 25 degrees Celsius (° C.) or standard room temperature. In some embodiments, compositions provided herein are thermally stable at about 45° C. In some embodiments, compositions provided herein are thermally stable at about −20° C. In some embodiments, compositions provided herein are thermally stable at about 2° C. to about 8° C. In some embodiments, compositions provided herein are thermally stable at a temperature of at least about −80° C., at least about −20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions provided herein are thermally stable for at least about 5 day, at least about 1 week, at least about 2 weeks, at least about 1 month, up to 3 months. In some embodiments, compositions provided herein are stored at a temperature of at least about 4° C. up to 37° C. for at least about 5 day, at least about 1 week, at least about 2 weeks, at least about 1 month, up to 3 months. In some embodiments, compositions provided herein are stored at a temperature of at least about 20° C. up to 25° C. for at least about 5 days, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### Pharmaceutical Compositions

Provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosage Forms

In some embodiments, a formulation of a PRR agonist provided herein is prepared in a single container for administration. In some embodiments, a formulation of a PRR agonist provided herein is prepared as two containers for administration, separating the PRR agonist from the nanoparticle carrier. As used herein, “container” includes vessel, vial, ampule, tube, cup, box, bottle, flask, jar, dish, well of a single-well or multi-well apparatus, reservoir, tank, an inhaler, a nasal spray apparatus, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

In some embodiments, the composition is lyophilized. In some embodiments, the composition is in a suspension, optionally a homogeneous suspension. In some embodiments, the composition is in an emulsion form. In some embodiments, pharmaceutical compositions provided here are in a from which allows for the composition to be administered to a subject. In some embodiments, the pharmaceutical composition is in the form of a solid, semi-solid, liquid or gas (aerosol).

In some embodiments, a composition provided herein is formulated for administration/for use in administration via an intratumoral, peritumoral, subcutaneous, intradermal, intramuscular, inhalation, intravenous, intraperitoneal, intracranial, intranasal, or intrathecal route. In some embodiments, the composition is provided in a dosage form which may be delivered via an inhaler, such as a solution, suspension, or powder, wherein the dosage form is formulated for delivery via an inhaler such as a metered-dose inhaler, a soft-mist inhaler, a nebulizer, or a dry powder inhaler. In some embodiments, the composition is provided in a dosage form which may be delivered via an intranasal spray device, such as a solution, suspension, or powder, wherein the dosage form is formulated for delivery via an intranasal spray device.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. Subjects include, without limitation, domesticate or farmed animals (including without limitation pigs, cows, horses, buffalo, pigs, ducks, geese, chicken, turkey, fish) as well as humans. Dosing may be for veterinary or human therapeutic uses. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition provided herein is formulated for administration/for use in administration via a subcutaneous, intradermal, intramuscular, inhalation, intravenous, intraperitoneal, intracranial, intranasal, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days.

In some embodiments, a single dose of a composition provided herein is administered to a subject. In some embodiments, a composition or pharmaceutical composition provided herein is administered to the subject by two doses. In some embodiments, a second dose of a composition or pharmaceutical composition provided herein is administered about 1 day, about 2 days, about 3 days, about 4 days, about 5 days, about 6 days, about 7 days, about 14 days, about 21 days, about 28 days, about 35 days, or about 56 days after the first dose. In some embodiments, a first dose is administered, and a second dose is administered about 14 days later, or about 21 days later, or about 28 days later, or about 35 days later, or about 42 days later, or about 49 days later, or about 56 days later, or about 63 days later, or about 70 days later, or about 77 days later, or about 84 days later. In some embodiments, the second dose is administered about 10-90 days following administration of the first dose, or about 15-85 days following administration of the first dose, or about 20-80 days following administration of the first dose, or about 25-75 days following administration of the first dose, or about 30-70 days following administration of the first dose, or about 35-65 days following administration of the first dose, or about 40-60 days following administration of the first dose.

In some embodiments, an additional, for example third or more, dose of a composition or pharmaceutical composition provided herein is administered to a subject. In some embodiments, the additional dose is administered about 1 month following administration of the second dose, about 2 months following administration of the second dose, about 3 months following administration of the second dose, about 4 months following administration of the second dose, about 5 months following administration of the second dose, about 6 months following administration of the second dose, about 7 months following administration of the second dose, about 8 months following administration of the second dose, about 9 months following administration of the second dose, about 10 months following administration of the second dose, about 11 months following administration of the second dose, about 12 months following administration of the second dose, about 13 months following administration of the second dose, about 14 months following administration of the second dose, about 15 months following administration of the second dose, about 16 months following administration of the second dose, about 17 months following administration of the second dose, or about 18 months following administration of the second dose.

In some embodiments, a single dose of a composition provided herein is administered to a subject via intranasal administration. In some embodiments, a composition or pharmaceutical composition provided herein is intranasally administered to the subject by two doses. In some embodiments, a second dose of a composition or pharmaceutical composition provided herein is administered about 24 hours, about 48 hours, or about 36 hours after the first dose. In some embodiments, a first dose is administered, and a second dose is administered about 1 day later, or about 2 days later, or about 3 days later, or about 4 days later, or about 5 days later, or about 6 days later, or about 7 days later, or about 8 days later, or about 9 days later, or about 10 days later, or about 11 days later, or about 12 days later or more. In some embodiments, the second dose is administered about 1-12 days following administration of the first dose, or about 1-3 days following administration of the first dose, or about 1-5 days following administration of the first dose, or about 1-7 days following administration of the first dose.

In some embodiments, an additional, for example, third or more, dose of a composition or pharmaceutical composition provided herein is administered to a subject via intranasal administration. In some embodiments, the additional dose is administered intranasally about In some embodiments, an additional dose of a composition or pharmaceutical composition provided herein is administered about 24 hours, about 48 hours, or about 36 hours after the second dose. In some embodiments, a first dose is administered, and a second dose is administered about 1 day later, or about 2 days later, or about 3 days later, or about 4 days later, or about 5 days later, or about 6 days later, or about 7 days later, or about 8 days later, or about 9 days later, or about 10 days later, or about 11 days later, or about 12 days later or more. In some embodiments, the additional dose is administered about 1-12 days following administration of the first dose, or about 1-3 days following administration of the first dose, or about 1-5 days following administration of the second dose, or about 1-7 days following administration of the second dose.

### Methods and Conditions

Provided herein are methods of treating a disease in a subject in need thereof. Further provided herein are methods of modulating an immune response in a subject in need thereof. Further provided herein are methods for treatment of cancer in a subject. In some embodiments, the methods comprise administering to a subject a composition provided herein. In some embodiments, the composition comprises a nanoparticle provided herein (e.g., any one of NP-1 to NP-37). In some embodiments, compositions provided herein for use in the methods provided herein comprise a nanoparticle and a nucleic acid provided herein. A nucleic acid provided herein can comprise, without limitation, one or more of a sequence that is at least 85%, 90%, 95%, 99%, or 100% identical to any one of SEQ ID NOS: 1-11.

In some embodiments, compositions provided herein are used for the treatment of a cancer. In some embodiments the cancer is a solid cancer or a hematopoietic cancer. In some embodiments, the solid cancer is a carcinoma, a melanoma, or a sarcoma. In some embodiments, the hematopoietic cancer is a lymphoma or a leukemia. In some embodiments, the cancer is a metastatic cancer. In some embodiments, the cancer is a skin cancer. In some embodiments, the skin cancer is a basal cell cancer, a melanoma, a Merkel cell cancer, a squamous cell carcinoma, a cutaneous lymphoma, a Kaposi sarcoma, or a skin adnexal cancer. In some embodiments, the subject has lung cancer. In some embodiments, the lung cancer is a non-small cell lung cancer (NSCLC) or a small cell lung cancer (SCLC). In some embodiments, the NSCLC is an adenocarcinoma, a squamous cell carcinoma, a large cell carcinoma, an adenosquamous carcinoma, or a sarcomatoid carcinoma. In some embodiments, the cancer is a pancreatic cancer. In some embodiments, the pancreatic cancer is a pancreatic adenocarcinoma or a pancreatic exocrine cancer. In some embodiments, the pancreatic cancer is a pancreatic neuroendocrine cancer, an islet cell cancer, or a pancreatic endocrine cancer. In some embodiments, the cancer is a prostate cancer.

In some embodiments, a composition provided herein is used for reduction of a tumor size. In some embodiments, a composition provided herein is used for reduction of a tumor volume. In some embodiments, a composition provided herein is used for reduction of a cancer recurrence. In some embodiments, a composition provided herein is used for reduction of tumor metastasis.

In some embodiments, the method for treatment of cancer comprises administration of a composition provided herein and radiation therapy. In some embodiments, the method for treatment of cancer comprises administration of a composition provided herein and irradiation. In some embodiments, the composition comprises a high atomic number (Z) element. In some aspects, the high-Z element of the embodiments is gold, silver, iodine, gallium, barium, iron, gadolinium, platinum, hafnium, bismuth or combinations thereof. In some embodiments, the composition comprises a nanoparticle lipid carrier comprising an inorganic particle. In some embodiments, the inorganic nanoparticle comprises iron oxide, optionally superparamagnetic iron oxide. In some embodiments, the inorganic particle comprises aluminum oxide, aluminum oxyhydroxide, iron oxide, titanium dioxide, or silicon dioxide. In some embodiments, the composition comprises a superparamagnetic agent. In some embodiments, the superparamagnetic agent comprises a metal oxides or sulfides which experience a magnetic domain. In some embodiments, the superparamagnetic agent comprises pure iron, magnetite, y—Fe₂O₃, Fe₃O₄, manganese ferrite, cobalt ferrite and nickel ferrite. In some embodiments, the nanoparticle lipid carrier comprises NP-1, NP-2, NP-3, NP-4, NP-6, NP-9, or NP-31. In some embodiments, the nanoparticle lipid carrier comprises any one of NP-1 to NP-37. In some embodiments, the composition further comprises a nucleic acid provided herein admixed to the nanoparticle lipid carrier. In some embodiments, the nanoparticle lipid carrier is not complexed to a nucleic acid. In some embodiments, the radiation therapy comprises low energy superficial kilovoltage, orthovoltage X-ray, high energy megavoltage (MV) photons, electron beam therapy (Linac), colbalt therapy, or brachytherapy. In some embodiments, the radiation therapy comprises administration of an X-ray, electron, gamma-ray, alpha or beta rays, or radioactive source (e.g., Au, CO, Celsium, and Iridium) localized into tumor tissue. In some embodiments the radiation is applied to localized superficial skin cancers, skin cancer with deep penetration, large or thick legions, or critical sites of a subject. Further provided herein are methods where the radiation dose is lower than the standard treatment dose due to the activity of the nanoparticle. Further provided herein are methods where the radiation is delivered by administering a radioactive isotope to the subject. Further provided herein is where the radioactive isotope is yttrium-90, or lutetium-177, or iodine-131, or samarium-153, or phosphorus-32. Further provided herein is where the isotope is delivered via a therapeutic. Further provided herein is where the isotope is bound to a monoclonal antibody. In some embodiments, the radiation is applied to a dermatological condition. In some embodiments, the dermatological condition is BCC, SCC, Bowen's disease, Erythroplasia, Angiosarcoma, Keratoacanthoma, Melanoma, Merkel cell carcinoma, Cutaneous lymphoma, Kaposi's sarcoma, or Fibrosarcoma. In some embodiments the dose is up to 35 Gy, up to 55 Gy, or from about 35 to about 55 Gy. In some embodiments, the radiotherapy comprises ionizing radiation administered at one time or as fractions over a period of time. In some embodiments, the schedule is over about 1 week to about 6 weeks. In some embodiments, the modality of irradiation comprises Grenz Rays, contact therapy, short source surface distance, superficial therapy, or orthovoltage therapy. In some embodiments the ionizing radiation is about 10-20 kV, 40-50 kV, 50-150 kV or 150-300 kV. In some embodiments, the ionizing radiation is at one or more energy levels from 1 kV to 10 MV photons or up to 300 MeV heavy ions. In some embodiments the treatment depth is <1 mm, 1-2 mm, >5 mm, or >5 mm and <2 cm. In some embodiments, the administration is to the brain, lung, liver, spleen, kidney, lymph node, small intestine, pancreas, blood, colon, stomach, breast, endometrium, prostate, testicle, ovary, skin, head and neck, esophagus, lymphatic, bone marrow or bone cancer cells.

Provided herein are methods of treating an infection in a subject. In some embodiments, the infection is a viral infection or a bacterial infection. In some embodiments, the viral infection is from a coronavirus. In some embodiments, the coronavirus is a Severe Acute Respiratory Syndrome Coronavirus-2 (SARS-CoV-2). In some embodiments, the coronavirus is MERS or SARS. In some embodiments, the viral infection is from an influenza virus. In some embodiments, the influenza virus is influenza A or influenza B. In some embodiments, the viral infection is from a Zika virus. In some embodiments, the viral infection is from a respiratory syncytial virus (RSV). In some embodiments, the viral infection is from hepatitis B. In some embodiments, the viral infection is from hepatitis C. In some embodiments, the viral infection is from a non-enveloped virus. In some embodiments, the non-enveloped virus is an enterovirus or a coxsackie virus. In some embodiments, the viral infection is from an enterovirus D68.

In some embodiments, compositions provided herein are used for the reduction of severity of an infection in a subject. In some embodiments, compositions provided herein provide for reduction of severity or duration of symptoms associated with an infection in a subject. In some embodiments, the infection is a viral infection. In some embodiments, the viral infection is from a coronavirus. In some embodiments, the coronavirus is SARS-CoV-2. In some embodiments, administration of a composition describes herein provides for reduction in the severity or duration of COVID-19 symptoms in a subject. In some embodiments, the coronavirus is MERS or SARS. In some embodiments, the viral infection is from an influenza virus. In some embodiments, the influenza virus is influenza A or influenza B. In some embodiments, the viral infection is from a Zika virus. In some embodiments, the viral infection is from a respiratory syncytial virus (RSV). In some embodiments, the viral infection is from hepatitis B. In some embodiments, the viral infection is from hepatitis C. In some embodiments, the viral infection is from Enterovirus D68.

Provided herein are methods of modulating an immune response in a subject, the methods comprise: administering to a subject a composition provided herein, thereby modulating an immune response to a protein. In some embodiments, compositions provided herein are used for augmentation of an immune response against a protein (e.g., a co-delivered antigen). In some embodiments, the protein is an antigen. In some embodiments, the antigen is a peptide or plurality of peptides. In some embodiments, the antigen is a nucleic acid. In some embodiments, the antigen is a complex mixture of antigens. In some embodiments, the protein is a cancer-associated protein, a viral protein, a bacterial protein, or a neoantigen. In some embodiments the viral protein an from a non-enveloped virus. In some embodiments, the non-enveloped virus is an enterovirus or a coxsackievirus. In some embodiments, the enterovirus is an enterovirus D 68.

Further provided herein are methods of prophylactically immunizing a subject for a cancer, the methods comprise: administering to a subject a composition provided herein and a cancer-associated protein, thereby immunizing the subject to the cancer expressing a cancer-associated protein. Further provided herein are methods of prophylactically immunizing a subject for an infection, the methods comprise: administering to a subject a composition provided herein and a microbial protein, thereby immunizing the subject to the microbial protein.

### Exemplary Embodiments

Provided herein are compositions, wherein the compositions comprise: a nanoparticle, wherein the nanoparticle comprises: a hydrophobic core, wherein lipids present in the hydrophobic core are in liquid phase at 25 degrees Celsius; and a hydrophilic surface; and a nucleic acid, wherein the nucleic acid comprises a region encoding a sequence that is at least 85% identical to any one of SEQ ID NOS: 1-11. Further provided herein are compositions, wherein the sequence is at least 90% identical to SEQ ID NO: 1. Further provided herein are compositions, wherein the sequence is at least 95% identical to SEQ ID NO: 1. Further provided herein are compositions, wherein the sequence is SEQ ID NO: 1. Further provided herein are compositions, wherein the sequence is at least 90% identical to SEQ ID NO: 2. Further provided herein are compositions, wherein the sequence is at least 95% identical to SEQ ID NO: 2. Further provided herein are compositions, wherein the sequence is SEQ ID NO: 2. Further provided herein are compositions, wherein the nucleic acid is present in an amount of up to about 10 μg. Further provided herein are compositions, wherein the nucleic acid is present in an amount of about 0.05, 0.1, 0.2, 0.5, 1, 5, 10, 12.5, 15, 20, 25, 50, 75, 100, 125, 150 or 200 μg. Further provided herein are compositions, wherein the nucleic acid is present in an amount of 0.05, 0.1, 0.2, 0.5, 1, 5, 10, 12.5, 15, 20, 25, 50, 75, 100, 125, 150, or 200 μg. Further provided herein are compositions, wherein the nanoparticle is characterized as having a z-average diameter particle size measurement of up to 100 nm in diameter when measured by dynamic light scattering. Further provided herein are compositions, wherein the nanoparticle is characterized as having a z-average diameter particle size measurement of 20 to 80 nm in diameter when measured by dynamic light scattering. Further provided herein are compositions, wherein the nanoparticle is characterized as having a z-average diameter particle size measurement of 40 to 80 nm in diameter when measured by dynamic light scattering. Further provided herein are compositions, wherein the nucleic acid is complexed to the nanoparticle. Further provided herein are compositions, wherein the nanoparticle comprises a membrane. Further provided herein are compositions, wherein the hydrophilic surface further comprises a cationic lipid. Further provided herein are compositions, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions, wherein the ratio of the cationic lipid to the nucleic acid is up to about 40:1. Further provided herein are compositions, wherein the ratio of the cationic lipid to the nucleic acid is up to about 8:1. Further provided herein are compositions, wherein the ratio of the cationic lipid to the nucleic acid is 25:1 to 100:1. Further provided herein are compositions, wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)— heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase. Further provided herein are compositions, wherein the oil is α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the nanoparticle further comprises an inorganic particle. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle further comprises a cationic lipid and an oil. Further provided herein are compositions, wherein the nanoparticle further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: a phosphate-terminated lipid; and a surfactant. Further provided herein are compositions, wherein the inorganic particle is coated with a capping ligand and the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the nanoparticle is in an aqueous solution.

Provided herein are compositions, wherein the compositions comprise: lipid nanoparticles, wherein the lipid nanoparticles are characterized as having a z-average diameter particle size measurement of 20 nm to 80 nm when measured using dynamic light scattering, and wherein the lipid nanoparticles comprise: a surface comprising cationic lipids; and a hydrophobic core, wherein the hydrophobic core comprises liquid oil, wherein lipids present in the hydrophobic core are in liquid phase at 25 degrees Celsius, and: a nucleic acid, wherein the nucleic acid comprises a pattern recognition receptor (PRR) agonist region, wherein the nucleic acid is present in an amount of up to 1 mg, and wherein the nucleic acid is in complex with the hydrophilic surface. Further provided herein are compositions, wherein the lipid nanoparticles further comprise an inorganic particle. Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the nucleic acid encodes for any one of SEQ ID NOS: 3-6. Further provided herein are compositions, wherein the nucleic acid encodes for any one of SEQ ID NOS: 7-11. Further provided herein are compositions, wherein the composition comprises a plurality of the nucleic acids coding different PRR agonists, optionally a TLR3 agonist and a RIG-I agonist. Further provided herein are compositions, wherein the nucleic acid encodes for different PRR agonists, optionally a TLR3 agonist and a RIG-I agonist. Further provided herein are compositions, wherein the PRR agonist is an agonist of TLR3, TLR7, or TLR8. Further provided herein are compositions, wherein the PRR agonist is a RIG-I-like receptor (RLR) agonist. Further provided herein are compositions, wherein the RLR is RIG-I. Further provided herein are compositions, wherein the nucleic acid encodes RNA. Further provided herein are compositions, wherein the nucleic acid encodes double-stranded RNA. Further provided herein are compositions, wherein the nucleic acid encodes single-stranded RNA. Further provided herein are compositions, wherein the lipid nanoparticles are characterized as having a z-average diameter particle size measurement of up to 100 nm in diameter when measured by dynamic light scattering. Further provided herein are compositions, wherein the lipid nanoparticles are characterized as having a z-average diameter particle size measurement of 20 to 80 nm in diameter when measured by dynamic light scattering. Further provided herein are compositions, wherein the lipid nanoparticles are characterized as having a z-average diameter particle size measurement of 40 to 80 nm in diameter when measured by dynamic light scattering. Further provided herein are compositions, wherein the lipid nanoparticles comprise a membrane. Further provided herein are compositions, wherein the hydrophilic surface comprises a cationic lipid. Further provided herein are compositions, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions, wherein the ratio of the cationic lipid to the nucleic acid is up to about 40:1. Further provided herein are compositions, wherein the ratio of the cationic lipid to the nucleic acid is up to about 8:1. Further provided herein are compositions, wherein the ratio of the cationic lipid to the nucleic acid is 25:1 to 100:1. Further provided herein are compositions, wherein the cationic lipid forms a lipid monolayer. Further provided herein are compositions, wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,N-trimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)— heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′Z,12Z,12′Z,12″Z,12′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid nanoparticles further comprise an inorganic particle. Further provided herein are compositions, wherein the inorganic particle is in a solid phase. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid nanoparticles comprise a cationic lipid and an oil. Further provided herein are compositions, wherein the lipid nanoparticles further comprise a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: a phosphate-terminated lipid; and a surfactant. Further provided herein are compositions, wherein each inorganic particle is coated with a capping ligand and the surfactant.

Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the nanoparticle is in an aqueous solution.

Provided herein are compositions, wherein the compositions comprise: a nanoparticle, wherein the nanoparticle comprises: a hydrophobic core, wherein the hydrophobic core is in solid phase at 25 degrees Celsius; and a hydrophilic surface; and a nucleic acid, wherein the nucleic acid comprises a region encoding a sequence that is at least 85% identical to any one of SEQ ID NOS: 1-11. Further provided herein are compositions, wherein the sequence is at least 90% identical to SEQ ID NO: 1. Further provided herein are compositions, wherein the sequence is at least 95% identical to SEQ ID NO: 1. Further provided herein are compositions, wherein the sequence is SEQ ID NO: 1. Further provided herein are compositions, wherein the sequence is at least 90% identical to SEQ ID NO: 2. Further provided herein are compositions, wherein the sequence is at least 95% identical to SEQ ID NO: 2. Further provided herein are compositions, wherein the sequence is SEQ ID NO: 2. Further provided herein are compositions, wherein the nucleic acid is present in an amount of up to about 10 μg. Further provided herein are compositions, wherein the nucleic acid is present in an amount of about 0.05, 0.1, 0.2, 0.5, 1, 5, 10, 12.5, 15, 20, 25, 50, 75, 100, 125, 150 or 200 μg. Further provided herein are compositions, wherein the nucleic acid is present in an amount of 0.05, 0.1, 0.2, 0.5, 1, 5, 10, 12.5, 15, 20, 25, 50, 75, 100, 125, 150, or 200 μg. Further provided herein are compositions, wherein the nanoparticle is characterized as having a z-average diameter particle size measurement of up to 100 nm in diameter when measured by dynamic light scattering. Further provided herein are compositions, wherein the nanoparticle is characterized as having a z-average diameter particle size measurement of 20 to 80 nm in diameter when measured by dynamic light scattering. Further provided herein are compositions, wherein the nanoparticle is characterized as having a z-average diameter particle size measurement of 40 to 80 nm in diameter when measured by dynamic light scattering. Further provided herein are compositions, wherein the nucleic acid is complexed to the nanoparticle. Further provided herein are compositions, wherein the nanoparticle comprises a membrane. Further provided herein are compositions, wherein the hydrophilic surface further comprises a cationic lipid. Further provided herein are compositions, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions, wherein the ratio of the cationic lipid to the nucleic acid is up to about 40:1. Further provided herein are compositions, wherein the ratio of the cationic lipid to the nucleic acid is up to about 8:1. Further provided herein are compositions, wherein the ratio of the cationic lipid to the nucleic acid is 25:1 to 100:1. Further provided herein are compositions, wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)— heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′Z,12Z,12′Z,12″Z,12′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises solanesol or glyceryl trimyristate-dynasan. Further provided herein are compositions, wherein the nanoparticle further comprises an inorganic particle. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle further comprises a cationic lipid and a solid hydrophobic core. Further provided herein are compositions, wherein the nanoparticle further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: a phosphate-terminated lipid; and a surfactant. Further provided herein are compositions, wherein the inorganic particle is coated with a capping ligand and the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the composition is lyophilized. Further provided herein are compositions, wherein the composition is dispersed in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the nanoparticle is in an aqueous solution.

Provided herein are compositions, wherein the compositions comprise: lipid nanoparticles, wherein the lipid nanoparticles are characterized as having a z-average diameter particle size measurement of 20 nm to 80 nm when measured using dynamic light scattering, and wherein the lipid nanoparticles comprise: a surface comprising cationic lipids; and a hydrophobic core, wherein the hydrophobic core is in solid phase at 25 degrees Celsius, and: a nucleic acid, wherein the nucleic acid comprises a pattern recognition receptor (PRR) agonist region, wherein the nucleic acid is present in an amount of up to 1 mg, and wherein the nucleic acid is in complex with the hydrophilic surface. Further provided herein are compositions, wherein the lipid nanoparticles further comprise an inorganic particle. Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the nucleic acid encodes for any one of SEQ ID NOS: 3-6. Further provided herein are compositions, wherein the nucleic acid encodes for any one of SEQ ID NOS: 7-11. Further provided herein are compositions, wherein the composition comprises a plurality of the nucleic acids coding different PRR agonists, optionally a TLR3 agonist and a RIG-I agonist. Further provided herein are compositions, wherein the nucleic acid encodes for different PRR agonists, optionally a TLR3 agonist and a RIG-I agonist. Further provided herein are compositions, wherein the PRR agonist is an agonist of TLR3, TLR7, or TLR8. Further provided herein are compositions, wherein the PRR agonist is a RIG-I-like receptor (RLR) agonist. Further provided herein are compositions, wherein the RLR is RIG-I. Further provided herein are compositions, wherein the nucleic acid encodes RNA. Further provided herein are compositions, wherein the nucleic acid encodes double-stranded RNA. Further provided herein are compositions, wherein the nucleic acid encodes single-stranded RNA. Further provided herein are compositions, wherein the lipid nanoparticles are characterized as having a z-average diameter particle size measurement of up to 100 nm in diameter when measured by dynamic light scattering. Further provided herein are compositions, wherein the lipid nanoparticles are characterized as having a z-average diameter particle size measurement of 20 to 80 nm in diameter when measured by dynamic light scattering. Further provided herein are compositions, wherein the lipid nanoparticles are characterized as having a z-average diameter particle size measurement of 40 to 80 nm in diameter when measured by dynamic light scattering. Further provided herein are compositions, wherein the lipid nanoparticles comprise a membrane. Further provided herein are compositions, wherein the hydrophilic surface comprises a cationic lipid. Further provided herein are compositions, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions, wherein the ratio of the cationic lipid to the nucleic acid is up to about 40:1. Further provided herein are compositions, wherein the ratio of the cationic lipid to the nucleic acid is up to about 8:1. Further provided herein are compositions, wherein the ratio of the cationic lipid to the nucleic acid is 25:1 to 100:1. Further provided herein are compositions, wherein the cationic lipid forms a lipid monolayer. Further provided herein are compositions, wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,N-trimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)— heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′Z,12Z,12′Z,12″Z,12′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises solanesol or glyceryl trimyristate-dynasan. Further provided herein are compositions, wherein the lipid nanoparticles further comprise an inorganic particle. Further provided herein are compositions, wherein the inorganic particle is in a solid phase. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid nanoparticles comprise a cationic lipid and an oil. Further provided herein are compositions, wherein the lipid nanoparticles further comprise a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: a phosphate-terminated lipid; and a surfactant. Further provided herein are compositions, wherein each inorganic particle is coated with a capping ligand and the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the nanoparticle is in an aqueous solution.

Provided herein are compositions, wherein the compositions comprise: a nanoparticle, wherein the nanoparticle comprises: a hydrophobic core comprising an inorganic particle; and a hydrophilic surface; and a nucleic acid, wherein the nucleic acid comprises a pattern recognition receptor (PRR) agonist region, wherein the nucleic acid is present in an amount of up to 1 mg or more, and wherein the nucleic acid is in complex with the hydrophilic surface. Further provided herein are compositions wherein the nucleic acid is present in an amount of above 5 μg to about 100 μg, or 5 μg to about 1 mg. Further provided herein are compositions wherein the nucleic acid is present in an amount of up to about 25, 50, 75, 100, 150, 175, 200 ng or more. Further provided herein are compositions wherein the nucleic acid is present in an amount of up to about 1 μg. Further provided herein are compositions wherein the nucleic acid is present in an amount of about 0.05, 0.1, 0.2, 0.5, 1, 5, 10, 12.5, 15, 25, 40, 50, 100, 125, 150, 175, 200, 250, 400, 500, 600, 700, 750, 1000 or more μg. Further provided herein are compositions wherein the nucleic acid is present in an amount of 0.05, 0.1, 0.2, 0.5, 1, 5, 10, 12.5, 15, 25, 40, 50, 100, 125, 150, 175, 200, 250, 400, 500, 600, 700, 750, 1000 or more μg. Further provided herein are compositions wherein the nucleic acid encodes for any one of SEQ ID NOS: 3-6. Further provided herein are compositions wherein the nucleic acid encodes for any one of SEQ ID NOS: 7-11. Further provided herein are compositions wherein the composition comprises a plurality of the nucleic acids coding different PRR agonists, optionally a TLR3 agonist and a RIG-I agonist. Further provided herein are compositions wherein the nucleic acid encodes for different PRR agonists, optionally a TLR3 agonist and a RIG-I agonist. Further provided herein are compositions wherein the PRR is a TLR3, TLR7, or TLR8. Further provided herein are compositions wherein the PRR is a TLR3. Further provided herein are compositions wherein the PRR is a RIG-I-like receptor (RLR). Further provided herein are compositions wherein the RLR is RIG-I. Further provided herein are compositions wherein the nucleic acid encodes RNA. Further provided herein are compositions wherein the nucleic acid encodes double-stranded RNA. Further provided herein are compositions wherein the nucleic acid encodes single-stranded RNA. Further provided herein are compositions wherein the nanoparticle is up to 100 nm in diameter. Further provided herein are compositions wherein the nanoparticle is 40 to 80 nm in diameter. Further provided herein are compositions wherein the nanoparticle comprises a membrane. Further provided herein are compositions wherein the hydrophilic surface comprises a cationic lipid. Further provided herein are compositions wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions wherein the ratio of the cationic lipid to the nucleic acid is up to about 40:1. Further provided herein are compositions wherein the ratio of the cationic lipid to the nucleic acid is up to about 8:1. Further provided herein are compositions wherein the ratio of the cationic lipid to the nucleic acid is 25:1 to 100:1. Further provided herein are compositions wherein the cationic lipid forms a lipid monolayer. Further provided herein are compositions wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)— heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions wherein the hydrophobic core comprises an oil. Further provided herein are compositions wherein the oil is in liquid phase. Further provided herein are compositions wherein the oil is α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. Further provided herein are compositions wherein the triglyceride is capric triglyceride, caprylic triglyceride, a caprylic and capric triglyceride, a triglyceride ester, or myristic acid triglycerin. Further provided herein are compositions wherein the inorganic particle is in a solid phase. Further provided herein are compositions wherein the inorganic particle comprises a metal. Further provided herein are compositions wherein the metal comprises a metal salt, a metal oxide, a metal hydroxide, or a metal phosphate. Further provided herein are compositions wherein the metal oxide comprises aluminum oxide, aluminum oxyhydroxide, iron oxide, titanium dioxide, or silicon dioxide. Further provided herein are compositions wherein the nanoparticle comprises a cationic lipid and an oil. Further provided herein are compositions wherein the nanoparticle further comprises a surfactant. Further provided herein are compositions wherein the surfactant is a hydrophobic surfactant. Further provided herein are compositions wherein the hydrophobic surfactant is sorbitan monolaurate, sorbitan monopalmitate, sorbitan monostearate, sorbitan monooleate, or sorbitan trioleate. Further provided herein are compositions wherein the surfactant is a hydrophilic surfactant. Further provided herein are compositions wherein the hydrophilic surfactant is a polysorbate. Further provided herein are compositions wherein the hydrophobic core further comprises: a phosphate-terminated lipid; and a surfactant. Further provided herein are compositions wherein each inorganic particle is coated with a capping ligand and the surfactant. Further provided herein are compositions wherein the phosphate-terminated lipid is trioctylphosphine oxide (TOPO). Further provided herein are compositions wherein the surfactant is a phosphorous-terminated surfactant, a carboxylate-terminated surfactant, a sulfate-terminated surfactant, or an amine-terminated surfactant. Further provided herein are compositions wherein the surfactant is distearyl phosphatidic acid (DSPA). Further provided herein are compositions wherein the composition is lyophilized. Further provided herein are compositions wherein the composition is in a liquid, semi-liquid, solution, propellant, or powder dosage form. Further provided herein are compositions wherein the composition is formulated as a suspension. Further provided herein are compositions wherein in the suspension is a homogeneous suspension. Further provided herein are compositions wherein the nanoparticle is in an aqueous solution. Provided herein are methods for treating cancer, comprising administering to a subject having cancer, a composition of any one of the embodiments provided herein; and administering irradiation to the subject. Further provided herein are methods wherein the cancer is a solid cancer or a hematopoietic cancer. Further provided herein are methods wherein the solid cancer is a melanoma, lung, liver, head and neck, or pancreatic cancer. Further provided herein are methods wherein the method provides for reduction in size and/or volume of the cancer. Further provided herein are methods wherein the method provides for reduction of tumor metastasis. Further provided herein are methods wherein the irradiation is after the administering of the composition. Further provided herein are methods wherein the irradiation comprises administering low energy superficial kilovoltage, orthovoltage X-ray, high energy megavoltage (MV) photons, electron beam therapy (Linac), colbalt therapy, or brachytherapy. Further provided herein are methods wherein the irradiation comprises administering an X-ray, electron, gamma-ray, alpha ray or beta ray. Further provided herein are methods wherein radiation is delivered by administering a radioactive isotope to the subject. Further provided herein are methods wherein the radioactive isotope is selected from yttrium-90, lutetium-177, iodine-131, samarium-153, and phosphorus-32. Further provided herein are methods wherein the radioactive isotope is delivered via a therapeutic. Further provided herein are methods wherein the radioactive isotope is bound to a monoclonal antibody. Further provided herein are methods wherein the radiation dose is lower than the standard treatment dose due to the activity of the nanoparticle. Further provided herein are methods wherein the irradiation is administered to localized superficial skin cancers, skin cancer with deep penetration, large or thick legions, or critical sites of the subject. Further provided herein are methods wherein the irradiation is applied to a dermatological condition of BCC, SCC, Bowen's disease, Erythroplasia, Angiosarcoma, Keratoacanthoma, Melanoma, Merkel cell carcinoma, Cutaneous lymphoma, Kaposi's sarcoma, or Fibrosarcoma. Further provided herein are methods wherein the irradiation dose is up to about 55 Gy. Further provided herein are methods wherein the irradiation is about 10-20 kV, 40-50 kV, 50-150 kV or 150-300 kV. Further provided herein are methods wherein the irradiation comprises a treatment depth of <1 mm, 1-2 mm, >5 mm, or >5 mm and <2 cm. Further provided herein are methods wherein the administering of the composition is intranasal, subcutaneous, intravenous, via inhalation, intramuscular, intratumoral, peritumoral, or intradermal. Further provided herein are methods wherein the administering of the composition is systemic. Further provided herein are methods wherein the administering of the composition is intratumoral. Further provided herein are methods wherein the administering is intranasal, subcutaneous, intravenous, via inhalation, intramuscular, intratumoral, peritumoral, or intradermal. Further provided herein are methods wherein the administering is systemic. Further provided herein are methods wherein the administering is intratumoral. Further provided herein are methods wherein administration of the composition and/or the irradiation is more than once.

Provided herein are compositions, wherein the compositions comprises: a nanoparticle, wherein the nanoparticle comprises: a hydrophobic core comprising an inorganic particle; and a hydrophilic surface; and a nucleic acid, wherein the nucleic acid comprises a region coding a sequence at least 85% identical to SEQ ID NO: 1. Further provided herein are compositions wherein the sequence is at least 90% identical to SEQ ID NO: 1. Further provided herein are compositions wherein the sequence is at least 95% identical to SEQ ID NO: 1. Further provided herein are compositions wherein the sequence is SEQ ID NO: 1. Further provided herein are compositions wherein the sequence is at least 90% identical to SEQ ID NO: 2. Further provided herein are compositions wherein the sequence is at least 95% identical to SEQ ID NO: 2. Further provided herein are compositions wherein the sequence is SEQ ID NO: 2. Further provided herein are compositions wherein the nucleic acid is present in an amount of up to about 10 μg. Further provided herein are compositions wherein the nucleic acid is present in an amount of about 0.05, 0.1, 0.2, 0.5, 1, 5, 10, 12.5, 15, 20, 25, 50, 75, 100, 125, 150 or 200 μg. Further provided herein are compositions wherein the nucleic acid is present in an amount of 0.05, 0.1, 0.2, 0.5, 1, 5, 10, 12.5, 15, 20, 25, 50, 75, 100, 125, 150, or 200 μg. Further provided herein are compositions wherein the nanoparticle is up to 100 nm in diameter. Further provided herein are compositions wherein the nanoparticle is 40 to 80 nm in diameter. Further provided herein are compositions wherein the nanoparticle comprises a membrane. Further provided herein are compositions wherein the hydrophobic core further comprises a cationic lipid. Further provided herein are compositions wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions wherein the ratio of the cationic lipid to the nucleic acid is up to about 40:1. Further provided herein are compositions wherein the ratio of the cationic lipid to the nucleic acid is up to about 8:1. Further provided herein are compositions wherein the ratio of the cationic lipid to the nucleic acid is 25:1 to 100:1. Further provided herein are compositions wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)— heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′9″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′Z,12Z,12′Z,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions wherein the hydrophobic core comprises an oil. Further provided herein are compositions wherein the oil is in liquid phase. Further provided herein are compositions wherein the oil is α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. Further provided herein are compositions wherein the triglyceride is capric triglyceride, caprylic triglyceride, a caprylic and capric triglyceride, a triglyceride ester, or myristic acid triglycerin. Further provided herein are compositions wherein the inorganic particle comprises a metal. Further provided herein are compositions wherein the metal comprises a metal salt, a metal oxide, a metal hydroxide, or a metal phosphate. Further provided herein are compositions wherein the metal oxide comprises aluminum oxide, aluminum oxyhydroxide, iron oxide, titanium dioxide, or silicon dioxide. Further provided herein are compositions wherein the nanoparticle further comprises a cationic lipid and an oil. Further provided herein are compositions wherein the nanoparticle further comprises a surfactant. Further provided herein are compositions wherein the surfactant is a hydrophobic surfactant. Further provided herein are compositions wherein the hydrophobic surfactant is sorbitan monolaurate, sorbitan monopalmitate, sorbitan monostearate, sorbitan monooleate, or sorbitan trioleate. Further provided herein are compositions wherein the surfactant is a hydrophilic surfactant. Further provided herein are compositions wherein the hydrophilic surfactant is a polysorbate. Further provided herein are compositions wherein he hydrophobic core further comprises: a phosphate-terminated lipid; and a surfactant. Further provided herein are compositions wherein the inorganic particle is coated with a capping ligand and the surfactant. Further provided herein are compositions wherein the phosphate-terminated lipid is trioctylphosphine oxide (TOPO). Further provided herein are compositions wherein the surfactant is a phosphorous-terminated surfactant, a carboxylate-terminated surfactant, a sulfate-terminated surfactant, or an amine-terminated surfactant. Further provided herein are compositions wherein the surfactant is distearyl phosphatidic acid (DSPA). Further provided herein are compositions wherein the composition is lyophilized. Further provided herein are compositions wherein the composition is in a liquid, semi-liquid, solution, propellant, or powder dosage form. Further provided herein are compositions wherein the composition is formulated as a suspension. Further provided herein are compositions wherein in the suspension is a homogeneous suspension. Further provided herein are compositions wherein the nanoparticle is in an aqueous solution. Provided herein are methods for treating cancer, comprising administering to a subject having cancer, a composition of any one of the embodiments provided herein; and administering radiation to the subject. Further provided herein are methods wherein the cancer is a solid cancer or a hematopoietic cancer. Further provided herein are methods wherein the solid cancer is a melanoma, lung, liver, head and neck, or pancreatic cancer. Further provided herein are methods wherein the method provides for reduction in size and/or volume of the cancer. Further provided herein are methods wherein the method provides for reduction of tumor metastasis. Further provided herein are methods wherein administered the radiation is after the administering of the composition. Further provided herein are methods wherein the irradiation comprises administering low energy superficial kilovoltage, orthovoltage X-ray, high energy megavoltage (MV) photons, electron beam therapy (Linac), colbalt therapy, or brachytherapy. Further provided herein are methods wherein administered the radiation comprises administering an X-ray, electron, gamma-ray, alpha ray or beta ray. Further provided herein are methods wherein radiation is delivered by administering a radioactive isotope to the subject. Further provided herein are methods wherein the radioactive isotope is selected from yttrium-90, lutetium-177, iodine-131, samarium-153, and phosphorus-32. Further provided herein are methods wherein the radioactive isotope is delivered via a therapeutic. Further provided herein are methods wherein the radioactive isotope is bound to a monoclonal antibody. Further provided herein are methods wherein the radiation is administered to localized superficial skin cancers, skin cancer with deep penetration, large or thick legions, or critical sites of the subject. Further provided herein are methods wherein the irradiation is applied to a dermatological condition of BCC, SCC, Bowen's disease, Erythroplasia, Angiosarcoma, Keratoacanthoma, Melanoma, Merkel cell carcinoma, Cutaneous lymphoma, Kaposi's sarcoma, or Fibrosarcoma. Further provided herein are methods wherein the irradiation dose is up to about 55 Gy. Further provided herein are methods wherein the irradiation is about 10-20 kV, 40-50 kV, 50-150 kV or 150-300 kV. Further provided herein are methods wherein administered the radiation comprises a treatment depth of <1 mm, 1-2 mm, >5 mm, or >5 mm and <2 cm Further provided herein are methods wherein the administering of the composition is intranasal, subcutaneous, intravenous, via inhalation, intramuscular, intratumoral, peritumoral, or intradermal. Further provided herein are methods wherein the administering of the composition is systemic. Further provided herein are methods wherein the administering of the composition is intratumoral. Further provided herein are methods wherein the administering is intranasal, subcutaneous, intravenous, via inhalation, intramuscular, intratumoral, peritumoral, or intradermal. Further provided herein are methods wherein the administering is systemic. Further provided herein are methods wherein the administering is intratumoral. Further provided herein are methods wherein administration of the composition and/or the irradiation is more than once.

Provided herein are suspensions, wherein the suspensions comprise a composition provided herein.

Provided herein are pharmaceutical compositions, wherein the pharmaceutical compositions comprise a composition provided herein; and a pharmaceutical excipient. Further provided herein are pharmaceutical compositions, wherein the pharmaceutical composition is formulated for intranasal administration or intratumoral administration.

Provided herein are methods for treatment of cancer in a subject, the methods comprising: administering to a subject having cancer, the composition provided herein, the suspension provided herein, or the pharmaceutical composition provided herein, thereby treating the cancer in the subject. Further provided herein are methods, wherein the administering is intranasal, subcutaneous, intravenous, via inhalation, intramuscular, intratumoral, peritumoral, or intradermal. Further provided herein are methods, wherein the administering is systemic. Further provided herein are methods, wherein the administering is intratumoral. Further provided herein are methods, wherein the cancer is a solid cancer. Further provided herein are methods, wherein the solid cancer is melanoma or lung cancer.

Provided herein are methods for treatment of cancer, comprising administering to a subject having cancer, a composition provided herein. Further provided herein is a method, wherein the administering is intranasal, subcutaneous, intravenous, via inhalation, or intramuscular. Further provided herein is a method, wherein the administering is systemic. Further provided herein is a method, wherein the administering is intratumoral. Further provided herein is a method, wherein the cancer is a skin cancer. Further provided herein is a method, wherein the skin cancer is melanoma. Further provided herein is a method, wherein the cancer is a solid cancer. Further provided herein are methods wherein the solid cancer is melanoma or lung cancer. Further provided herein is a method, wherein the administration is more than once. Further provided herein is a method, wherein the administration is two, three, four or more times.

Provided herein are methods for treatment of an infection in a subject, the methods comprising: administering to a subject having an infection, the composition provided herein, the suspension provided herein, or the pharmaceutical composition provided herein, thereby treating the infection in the subject. Further provided herein are methods, wherein the administering is intranasal, subcutaneous, intravenous, via inhalation, or intramuscular. Further provided herein are methods, wherein the infection is a viral infection. Further provided herein are methods, wherein the viral infection is a coronavirus infection. Further provided herein are methods, wherein the coronavirus infection is a SARS-COV-2 infection. Further provided herein are methods, wherein the viral infection is a respiratory syncytial virus (RSV) infection, a hepatitis B infection, a hepatitis C infection, enterovirus D68, or an influenza infection.

Provided herein is a method for treatment of an infection, comprising administering to a subject having an infection, the composition provided herein. Further provided herein is a method, wherein the administering is intranasal, subcutaneous, intravenous, via inhalation, or intramuscular. Further provided herein is a method, wherein the infection is a viral infection. Further provided herein is a method, wherein the infection is a coronavirus infection. Further provided herein is a method, wherein the coronavirus is a SARS-CoV-2. Further provided herein is a method, wherein the administration is more than once. Further provided herein is a method, wherein the administration is two, three, four or more times.

Provided herein are methods for the reduction of severity of an infection in a subject, the methods comprising: administering to a subject, the composition provided herein, the suspension provided herein, or the pharmaceutical composition provided herein, thereby reducing the severity of the infection. Further provided herein are methods, wherein the administering is intranasal, subcutaneous, intravenous, via inhalation, or intramuscular. Further provided herein are methods, wherein the infection is a viral infection. Further provided herein are methods, wherein the viral infection is a coronavirus infection. Further provided herein are methods, wherein the coronavirus infection is a SARS-COV-2 infection. Further provided herein are methods, wherein the viral infection is an RSV infection, a hepatitis B infection, a hepatitis C infection, enterovirus D68 infection, or an influenza infection.

Provided herein is a method for reduction of severity of an infection, comprising administering to a subject, the composition provided herein. Further provided herein is a method, wherein the administering is intranasal, subcutaneous, intravenous, via inhalation, or intramuscular. Further provided herein is a method, wherein the infection is a viral infection. Further provided herein is a method, wherein the infection is a coronavirus infection. Further provided herein is a method, wherein the coronavirus is a SARS-COV-2. Provided herein is a method for reducing tumor size, comprising: intratumorally administering to a subject having cancer the composition provided herein. Provided herein is a method for increasing monocyte recruitment to a cancer, comprising: intratumorally administering to a subject having cancer the composition provided herein. Provided herein is a method for increasing cDC1 activation in a subject, comprising: administering to a subject the composition provided herein. Further provided herein is a method the subject has cancer. Further provided herein is a method, wherein the skin cancer is melanoma. Further provided herein is a method, wherein the administration is more than once. Further provided herein is a method, wherein the administration is two, three, four or more times. Further provided herein is a method, wherein the viral infection is RSV, hepatitis B, hepatitis C, Enterovirus D68, or influenza.

Provided herein are methods for reduction of severity of an infection, comprising intranasally administering to a subject a composition comprising PRR agonist. Further provided herein is a method, wherein the infection is a viral infection. Further provided herein is a method the infection is a coronavirus infection. Further provided herein is a method, wherein the coronavirus is a SARS-COV-2. Further provided herein is a method, wherein the PRR agonist is a nucleic acid. Further provided herein is a method, wherein the nucleic acid is RIG-I agonist. Further provided herein is a method, wherein the nucleic acid is TLR3 agonist. Further provided herein is a method, wherein the nucleic acid comprises a region coding any one of SEQ ID NOS: 1-11. Further provided herein is a method, wherein the nucleic acid comprises a region coding a sequence at least 85% identical to SEQ ID NO: 1. the nucleic acid comprises a region coding SEQ ID NO: 1. Further provided herein is a method, wherein the nucleic acid comprises a region coding a sequence at least 85% identical to SEQ ID NO: 2. Further provided herein is a method, wherein the nucleic acid comprises a region coding SEQ ID NO: 2. Further provided herein is a method, wherein the composition further comprises a nanoparticle. Further provided herein is a method, wherein the nanoparticle comprises NP-1 or 23. Further provided herein is a method, wherein the nanoparticle is any one of NP-1 to NP-31. Further provided herein is a method, wherein the nanoparticle comprises a hydrophobic core comprising an inorganic particle and a liquid oil. Further provided herein is a method, wherein the nanoparticle further comprises a cationic lipid. Further provided herein is a method, wherein the nucleic acid is admixed with the nanoparticle. Further provided herein is a method, wherein the administration is prior to infection or preventative. Further provided herein is a method, wherein the administration is more than once. Further provided herein is a method, wherein the administration is two, three, four or more times. Further provided herein is a method, wherein the viral infection is from a non-enveloped virus. Further provided herein is a method, wherein the viral infection is an RSV infection, a hepatitis B infection, a hepatitis C infection, an enterovirus D68 infection, or an influenza infection.

Provided herein is a method for increasing monocyte recruitment to augment an immune response, optionally for treatment or prevention of cancer or an infection comprising administering to a subject a composition provided herein. Provided herein is a method for augmenting an immune response to a co-delivered antigen, comprising administering to a subject the composition provided herein. In some embodiments, the antigen is co-administered with the composition provided herein. Further provided herein is a method, wherein the administering is intranasal, subcutaneous, intravenous, via inhalation, or intramuscular. Further provided herein is a method, wherein the antigen is a protein, a peptide or peptides, a nucleic acid, or a complex mixture of antigens. Further provided herein is a method, wherein the administration is more than once. Further provided herein is a method, wherein the administration is two, three, four or more times.

Provided herein are methods for increasing monocyte recruitment to augment an immune response in a subject, optionally for treatment or prevention of cancer or an infection, the method comprising: intratumorally administering to a subject the composition provided herein, thereby increasing monocyte recruitment to augment an immune response in a subject.

Provided herein are methods for treatment of cancer, comprising: administering to a subject having cancer a composition, wherein the composition comprises a nanoparticle, wherein the nanoparticle comprises: a hydrophobic core comprising an inorganic particle; and a hydrophilic surface; and administering radiation to the subject. Further provided herein are methods wherein the cancer is a solid cancer or a hematopoietic cancer. Further provided herein are methods wherein the solid cancer is a melanoma, lung, liver, head and neck, or pancreatic cancer. Further provided herein are methods wherein the method provides for reduction in size and/or volume of the cancer. Further provided herein are methods wherein the method provides for reduction of tumor metastasis. Further provided herein are methods wherein administered the radiation is after the administering of the composition. Further provided herein are methods wherein the inorganic particle is a high atomic number element. Further provided herein are methods wherein the high atomic number element is gold, silver, iodine, gallium, barium, iron, gadolinium, platinum, hafnium, bismuth or combinations thereof. Further provided herein are methods wherein the inorganic particle iron oxide, optionally superparamagnetic iron oxide. Further provided herein are methods wherein the inorganic particle comprises aluminum oxide, aluminum oxyhydroxide, iron oxide, titanium dioxide, or silicon dioxide. Further provided herein are methods wherein the inorganic particle comprises pure iron, magnetite, y—Fe₂O₃, Fe₃O₄, manganese ferrite, cobalt ferrite or nickel ferrite. Further provided herein are methods wherein the nanoparticle comprises NP-1, NP-2, NP-3, NP-4, NP-6, NP-9, or NP-31. Further provided herein are methods wherein the nanoparticle comprises NP-1. Further provided herein are methods wherein administered the radiation comprises administering low energy superficial kilovoltage, orthovoltage X-ray, high energy megavoltage (MV) photons, electron beam therapy (Linac), colbalt therapy, or brachytherapy. Further provided herein are methods wherein the irradiation comprises administering an X-ray, electron, gamma-ray, alpha ray or beta ray. Further provided herein are methods wherein radiation is delivered by administering a radioactive isotope to the subject. Further provided herein are methods wherein the radioactive isotope is selected from yttrium-90, lutetium-177, iodine-131, samarium-153, and phosphorus-32. Further provided herein are methods wherein the radioactive isotope is delivered via a therapeutic. Further provided herein are methods wherein the radioactive isotope is bound to a monoclonal antibody. Further provided herein are methods wherein the irradiation is administered to localized superficial skin cancers, skin cancer with deep penetration, large or thick legions, or critical sites of the subject. Further provided herein are methods wherein the irradiation is applied to a dermatological condition of BCC, SCC, Bowen's disease, Erythroplasia, Angiosarcoma, Keratoacanthoma, Melanoma, Merkel cell carcinoma, Cutaneous lymphoma, Kaposi's sarcoma, or Fibrosarcoma. Further provided herein are methods wherein the radiation dose is up to about 55 Gy. Further provided herein are methods wherein the radiation is about 10-20 kV, 40-50 kV, 50-150 kV or 150-300 kV. Further provided herein are methods wherein the irradiation comprises a treatment depth of <1 mm, 1-2 mm, >5 mm, or >5 mm and <2 cm. Further provided herein are methods wherein the irradiation is administered to the brain, lung, liver, spleen, kidney, lymph node, small intestine, pancreas, blood, colon, stomach, breast, endometrium, prostate, testicle, ovary, skin, head and neck, esophagus, lymphatic, bone marrow or bone cancer cells. Further provided herein are methods wherein the administering of the composition is intranasal, subcutaneous, intravenous, via inhalation, intramuscular, intratumoral, peritumoral, or intradermal. Further provided herein are methods wherein the administering of the composition is systemic. Further provided herein are methods wherein the administering of the composition is intratumoral.

Provided herein are methods for treatment of cancer, comprising: administering to a subject having cancer a composition, wherein the composition comprises: a nanoparticle, wherein the nanoparticle comprises: a hydrophobic core comprising an inorganic particle; and a hydrophilic surface; and a nucleic acid, wherein the nucleic acid comprises a pattern recognition receptor (PRR) agonist region; and administering irradiation to the subject. Further provided herein are methods wherein the cancer is a solid cancer or a hematopoietic cancer. Further provided herein are methods wherein the solid cancer is a melanoma, lung, liver, head and neck, or pancreatic cancer. Further provided herein are methods wherein the method provides for reduction in size and/or volume of the cancer. Further provided herein are methods wherein the method provides for reduction of tumor metastasis. Further provided herein are methods wherein the irradiation is after the administering of the composition. Further provided herein are methods wherein the inorganic particle is a high atomic number element. Further provided herein are methods wherein the high atomic number element is gold, silver, iodine, gallium, barium, iron, gadolinium, platinum, hafnium, bismuth or combinations thereof. Further provided herein are methods wherein the inorganic particle iron oxide, optionally superparamagnetic iron oxide Further provided herein are methods wherein the inorganic particle comprises aluminum oxide, aluminum oxyhydroxide, iron oxide, titanium dioxide, or silicon dioxide. Further provided herein are methods wherein the inorganic particle comprises pure iron, magnetite, y—Fe₂O₃, Fe₃O₄, manganese ferrite, cobalt ferrite or nickel ferrite. Further provided herein are methods wherein the nanoparticle comprises NP-1, NP-2, NP-3, NP-4, NP-6, NP-9, or NP-31. Further provided herein are methods wherein the nanoparticle comprises NP-1. Further provided herein are methods wherein administered the radiation comprises administering low energy superficial kilovoltage, orthovoltage X-ray, high energy megavoltage (MV) photons, electron beam therapy (Linac), colbalt therapy, or brachytherapy. Further provided herein are methods wherein the irradiation comprises administering an X-ray, electron, gamma-ray, alpha ray or beta ray. Further provided herein are methods wherein radiation is delivered by administering a radioactive isotope to the subject. Further provided herein are methods wherein the radioactive isotope is selected from yttrium-90, lutetium-177, iodine-131, samarium-153, and phosphorus-32. Further provided herein are methods wherein the radioactive isotope is delivered via a therapeutic. Further provided herein are methods wherein the radioactive isotope is bound to a monoclonal antibody. Further provided herein are methods wherein the radiation is administered to localized superficial skin cancers, skin cancer with deep penetration, large or thick legions, or critical sites of the subject. Further provided herein are methods wherein the irradiation is applied to a dermatological condition of BCC, SCC, Bowen's disease, Erythroplasia, Angiosarcoma, Keratoacanthoma, Melanoma, Merkel cell carcinoma, Cutaneous lymphoma, Kaposi's sarcoma, or Fibrosarcoma. Further provided herein are methods wherein the radiation dose is up to about 55 Gy. Further provided herein are methods wherein the radiation is about 10-20 kV, 40-50 kV, 50-150 kV or 150-300 kV. Further provided herein are methods wherein the irradiation comprises a treatment depth of <1 mm, 1-2 mm, >5 mm, or >5 mm and <2 cm. Further provided herein are methods wherein the irradiation is administered to the brain, lung, liver, spleen, kidney, lymph node, small intestine, pancreas, blood, colon, stomach, breast, endometrium, prostate, testicle, ovary, skin, head and neck, esophagus, lymphatic, bone marrow or bone cancer cells. Further provided herein are methods wherein the nucleic acid comprises a sequence having at least 85% identity to any one of SEQ ID NOS: 1-11. Further provided herein are methods wherein the nucleic acid encodes for any one of SEQ ID NOS: 1-2. Further provided herein are methods wherein the nucleic acid encodes for any one of SEQ ID NOS: 3-6. Further provided herein are methods wherein the nucleic acid encodes for any one of SEQ ID NOS: 7-11. Further provided herein are methods wherein the composition comprises a plurality of the nucleic acids coding different PRR agonists, optionally a TLR3 agonist and a RIG-I agonist. Further provided herein are methods wherein the nucleic acid encodes for different PRR agonists, optionally a TLR3 agonist and a RIG-I agonist. Further provided herein are methods wherein the PRR agonist is an agonist of TLR3, TLR7, or TLR8. Further provided herein are methods wherein the PRR agonist is a RIG-I-like receptor (RLR) agonist. Further provided herein are methods wherein the RLR is RIG-I. Further provided herein are methods wherein the nucleic acid encodes RNA. Further provided herein are methods wherein the nucleic acid encodes double-stranded RNA. Further provided herein are methods wherein the nucleic acid encodes single-stranded RNA. Further provided herein are methods wherein the nanoparticle is up to 100 nm in diameter. Further provided herein are methods wherein the nanoparticle is 40 to 80 nm in diameter. Further provided herein are methods wherein the administering of the composition is intranasal, subcutaneous, intravenous, via inhalation, intramuscular, intratumoral, peritumoral, or intradermal. Further provided herein are methods wherein the administering of the composition is systemic. Further provided herein are methods wherein the administering of the composition is intratumoral.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: In-Vitro Transcription of RIG-I Agonist RNA

The following describes the generation of a RIG-I agonist RNA sequence. For the transcription reaction, the ingredients in Table 3 were used, with a MEGAshortscript kit, assembled at room temperature.

TABLE 3

| Ingredient | Volume |
|---|---|
| T7 10x transcription reaction buffer | 2 μL |
| ATP solution | 2 μL |
| CTP solution | 2 μL |
| GTP solution | 2 μL |
| UTP solution | 2 μL |
| T7 enzyme mix | 2 μL |
| DNA template | 2 μL |
| Nuclease free water | μL |

For the DNA template, a DNA plasmid was used. The DNA plasmid has a kanamycin resistance gene, lacZ gene reporter, and a T7 promoter for initiation of transcription. The region for transcription includes the following DNA sequence, and its reverse complements as listed in Table 4. For SEQ ID NO: 12, the T7 promoter begins with the TAATA sequence, the TCTAGA is the cut site for XbaI.

TABLE 4

| SEQ ID | |
|---|---|
| NO: | SEQUENCE |
| 12 | DNA 5′ |
| | GGAACTAATACGACTCACTATAGGCCATCCTGTTTTTTTCCCTTTTTTTT |
| | TTTCTTTTTTTTTTTTTTTTTTTTTTTTTTTTTTTTTTCTCCTTTTTTTTTCCT |
| | CTTTTTTTCCTTTTCTTTCCTTT TCTAGAGTTCC 3′ |
| 13 | 3′ |
| | CCTTGATTATGCTGAGTGATATCCGGTAGGACAAAAAAAGGGAAAAAA |
| | AAAAAGAAAAAAAAAAAAAAAAAAAAAAAAAAAAAAAAAAGAGGAA |
| | AAAAAAAGGAGAAAAAAAGGAAAAGAAAGGAAAAGATCTCAAGG 5′ |

The reaction mixture was incubated at 30 degrees Celsius for overnight. 1 μL Turbo DNAse was added to remove DNA template, followed by incubation at 37 degrees Celsius for 15 minutes. To purify RNA by phenol-chloroform extraction and ethanol precipitation, the following steps were taken. Next, 115 μL of nuclease free water and 15 μL ammonium acetate stop solution (Megashortscript kit) were added. The following were added: an equal volume (150 μL) phenol and (150 μL) chloroform, mixed and left to settle for a few minutes. Next, the aqueous phase was recovered and transferred to a new tube. To the new tube, 300 μL of 100% ethanol was added, then the mixture was chilled at minus 20 degrees Celsius for at least 15 minutes. After centrifugation at 4 degrees Celsius for 15 minus at high RPM, an RNA pellet was formed. The supernatant was removed, and the pellet was resuspended in 50 μL of water. The resultant RNA has a sequence of SEQ ID NO: 2.

### Example 2: Manufacture and Stability of NP-1

Manufacture of NP-1. NP-1 particles comprise 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID), 0.2 mg Fe/ml 12 nm oleic acid-coated iron oxide nanoparticles (ImagionBio) and 10 mM sodium citrate dihydrate (Fisher Chemical). 1 ml of 20 mg Fe/ml 12 nm diameter oleic acid-coated iron oxide nanoparticles in chloroform (ImagionBio, lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M1 10P microfluidizer (Microfluidics) at 30,000 psi equipped with a F12Y 75 μm diamond interaction chamber and an auxiliary H30Z-200 m ceramic interaction chamber until the z-average hydrodynamic diameter—measured by dynamic light scattering (Malvern Zetasizer Nano S) -reached 40-80 nm with a 0.1-0.25 polydispersity index (PDI). The microfluidized carrier was terminally filtered with a 200 nm pore-size polyethersulfone (PES) filter and stored at 2-8 degrees Celsius (° C.). Iron concentration was determined by ICP-OES. DOTAP and Squalene concentration were measured by RP-HPLC.

Manufacture of NP-3. NP-3 particles comprise 37.5 mg/mi Miglyol 812 N (101 Oleo GmbH), 37 mg/ml Span® 60 (Millipore Sigma), 37 mg/ml Tween® 80 (Fisher Chemical), 30 mg/ml DOTAP chloride (LIPOID), 0.2 mg Fe/ml 15 nm oleic acid-coated iron oxide nanoparticles (ImagionBio) and 10 mM sodium citrate dihydrate (Fisher Chemical). 1 ml of 20 mg Fe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65 degree Celsius (° C.) water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M1 10P microfluidizer (Microfluidics) at 30,000 psi equipped with a F12Y 75 μm diamond interaction chamber and an auxiliary H30Z-200 m ceramic interaction chamber until the z-average hydrodynamic diameter—measured by dynamic light scattering (Malvern Zetasizer Nano S) -reached 40-80 nm with a 0.1-0.3 polydispersity index (PDI). The microfluidized nanoparticle was terminally filtered with a 200 nm pore-size polyethersulfone (PES) filter and stored at 2-8° C. Iron concentration was determined by ICP-OES. DOTAP concentration was measured by RP-HPLC.

iii. Manufacture of NP-30. A lipid carrier without providing inorganic core particles in the core was generated having 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID) and 10 mM sodium citrate. To a 200 ml beaker 3.75 grams squalene, 3.7 grams span 60, and 3.0 grams DOTAP were added to produce the oil phase. The oil phase was sonicated for 45 minutes in a 65 degrees Celsius water bath. Separately, the aqueous phase was prepared by dissolving 19.5 grams Tween 80 in 500 ml of 10 mM sodium citrate buffer prepared in nuclease free water. 96 ml of the aqueous phase was transferred to a separate glass bottle and heated to 65 degrees Celsius for 30 minutes. The oil phase was mixed with the 96 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 m ceramic interaction chamber until the z-average hydrodynamic diameter—measured by dynamic light scattering (Malvern Zetasizer Nano S)-reached 40-80 nm with a 0.1-0.3 polydispersity index (PDI). The microfluidized nanoparticle without inorganic core formulation was terminally filtered with a 200 nm pore-size polyethersulfone (PES) filter and stored at 2 to 8 degrees Celsius. DOTAP and Squalene concentration were measured by RP-HPLC.

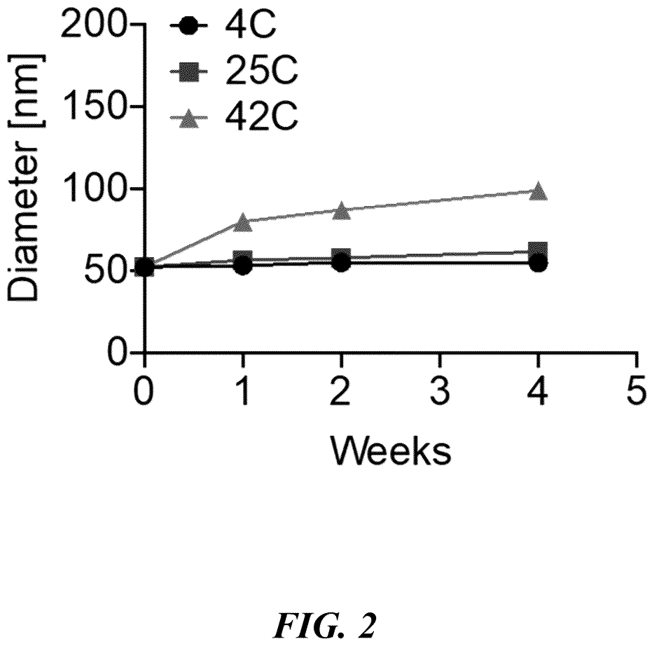

Stability. A nanoparticle according to NP-1 was placed into a stability chamber at the indicated temperatures. The stability was determined by particle size measurement using dynamic light scattering. The results show that the NP-1 formulation formed a stable colloid when stored at 4, 25 and 42 degrees Celsius. Time measurements were taken over 4 weeks. As shown in FIG. 2, the range of nanoparticle size was about 50-100 nm in diameter, and closer to 40-60 nm in diameter for the 4 and 25 degrees Celsius conditions over time.

### Example 3: PRR Agonist and Nanoparticle Complexing

The following describes PRR agonist and nanoparticle complexing at various ratios. Complexing is indicated by survival of RNase challenge. Briefly, complexes of a RIG-I agonist, an RNA having SEQ ID NO: 2, with nanoparticle NP-1 were generated, having nitrogen-to-phosphate (N:P) molar ratio of 16, 4, 1, 0.25, and 0.06. The N:P ratio is the ratio of positively charged nitrogens (N) on NP-1 formulation to negatively charged phosphates on the RNA (P). The RNA concentration is measured by nanodrop. N is determined by the amount of cationic lipid in the nanoparticle, and DOTAP in the case of NP-1. The complexed reagents were incubated on ice for 30 minutes. Half the complex was run on an RNA gel electrophoresis to assess unbound RIG-I agonist, and visually assessed following an image capture. The image showed no naked nucleic acid present when the RIG-I agonist nucleic acid and nanoparticle were mixed at N:P ratios >1 (data not shown). Thus, the RIG-I agonist was able to complex with the nanoparticle.

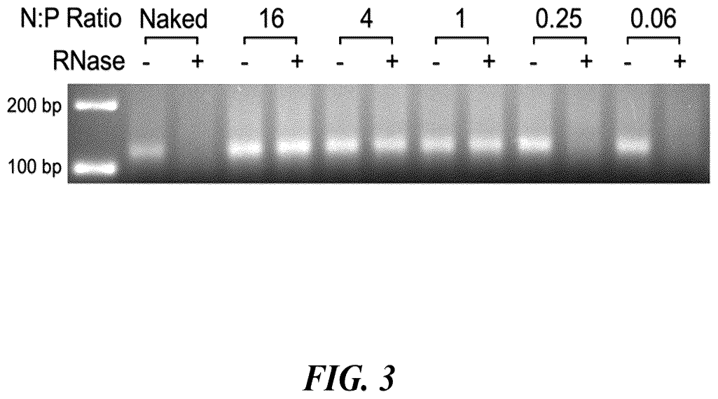

To the remaining complex sample, RNase challenge was performed, followed by treatment with proteinase to quench the reaction. The RNA was extracted from the surviving complex using a phenol chloroform extraction. The aqueous phase was mixed with glyoxal running buffer and run on an RNA gel electrophoresis and visually assessed following an image capture. See FIG. 3. The results showed that the RIG-I agonist nucleic acid was protected from RNase activity at all levels of N:P ratio tested. In contrast, control naked RNA was degraded by the RNase as can be seen in lane 2 of FIG. 3.

### Example 4: Nanoparticle to PRR Agonist Molar Ratio Analysis

Various conditions were analyzed to assess the impact of nanoparticle to PRR agonist molar ratios on downstream PRR signaling activity. The PRR agonist was a RIG-I agonist having SEQ ID NO: 2. The nanoparticle was NP-1.

A549-Dual cells were used. A549-Dual cells are adherent epithelial cells that have been derived from the human A549 μlung carcinoma cell line by stable integration of two inducible reporter constructs. The A549 cell line is a well-characterized cellular model for asthma, allergies and respiratory infections. A549-Dual cells express a secreted embryonic alkaline phosphatase (SEAP) reporter gene under the control of the IFN-β minimal promoter fused to five NF-κB binding sites. A549-Dual cells also express the Lucia luciferase gene, which encodes a secreted luciferase, under the control of an ISG54 minimal promoter in conjunction with five IFN-stimulated response elements. As a result, A549-Dual cells allow to simultaneously study the NF-κB pathway, by assessing the activity of SEAP, and the interferon regulatory factor (IRF) pathway, by monitoring the activity of Lucia luciferase. Both reporter proteins are readily measurable in the cell culture supernatant when using QUANTI-Blue, a SEAP detection reagent, and QUANTI-Luc, a Lucia luciferase detection reagent.

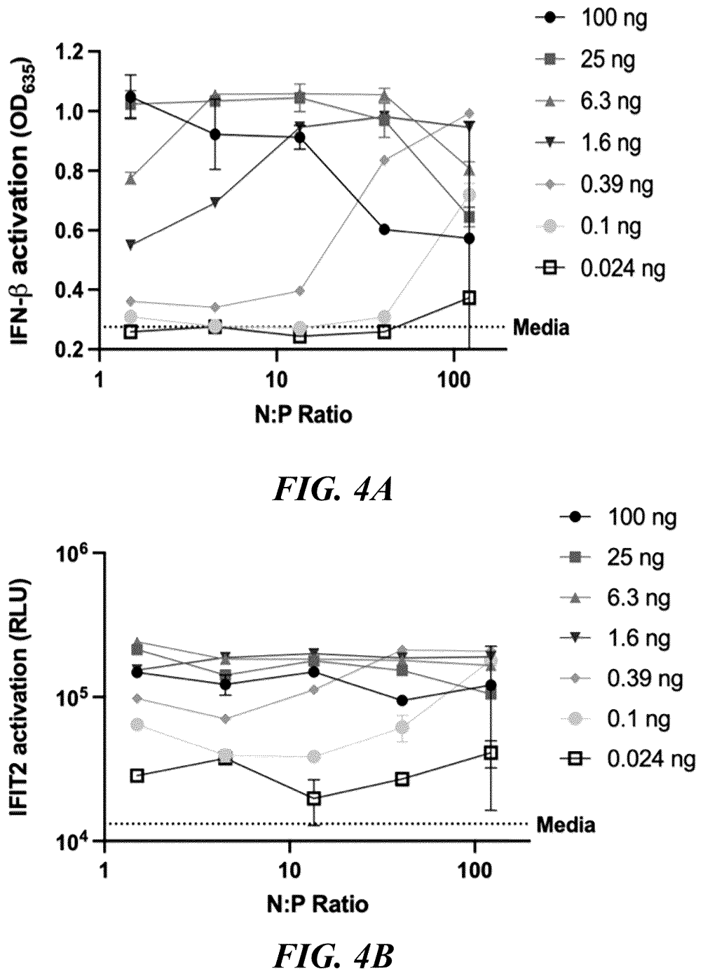

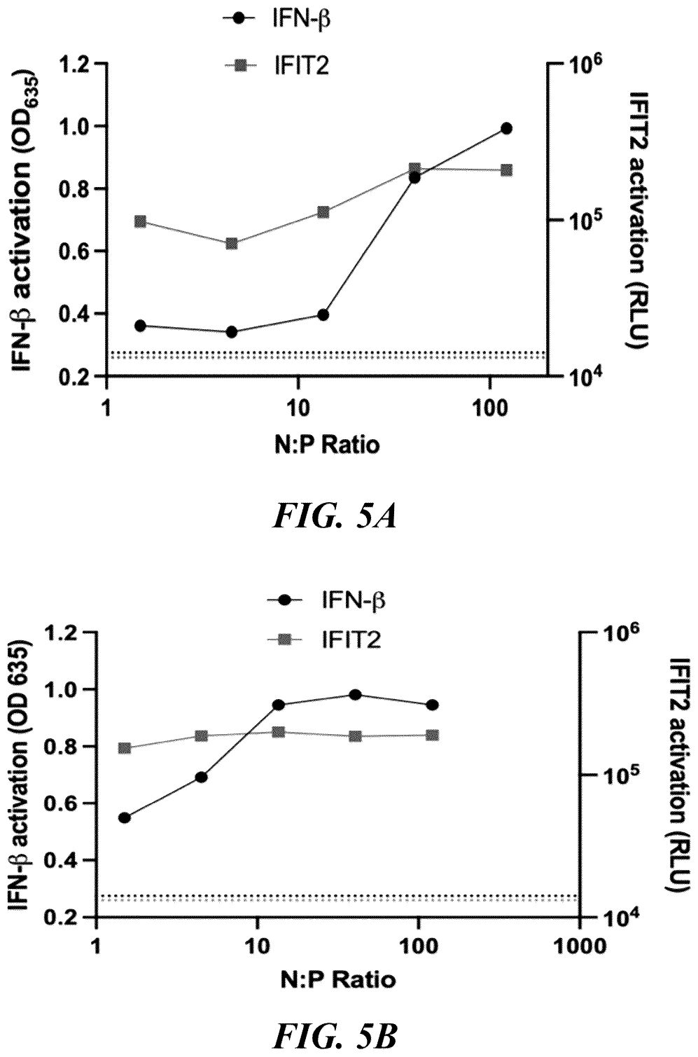

RIG-I agonist having SEQ ID NO: 2 was complexed for 30 minutes with NP-1 at N:P ratios ranging from 0.5 to 121, to form RNA-NP complexes. RNA amounts were 0.024, 0.1, 0.39, 1.6, 6.3, 25, and 100 ng. The RNA-NP complexes were transfected in cells with Opti-MEM for 4 hours. Media was removed and replaced with complete DMEM overnight. IFN-β and IFIT2 activity was monitored 20 hours later using Quanti-Blue and Quanti-Luc kits, respectively. Cell supernatants were monitored for IFN-β (FIG. 4A) and IFIT2 (FIG. 4B) activation by the presence of SEAP or luciferase in the supernatant, respectively. Further, a comparison of IFN-β and IFIT2 responses in A549-Dual cells from FIGS. 4A-4B at either 0.39 ng (FIG. 5A) or 1.6 ng (FIG. 5B) RNA levels was generated. Dotted lines indicate media alone control. The results showed that the RIG-I agonist having SEQ ID NO: 2 in complex with NP-1 upregulated IFN-β and IFIT2 production down to 0.1 ng of RNA. As the dose of the RNA decreases, higher N:P ratios were associated with upregulated IFN-β and IFIT2 production. In addition, the RNA-NP complex upregulated IFIT2 production at lower doses than those required to upregulate IFN-β production.

### Example 5: Nanoparticle to PRR Agonist Molar Ratio and Metal Analysis

Various conditions were analyzed to assess nanoparticle to PRR agonist molar ratios and metal selection on downstream PRR signaling activity. Assays were performed following a similar framework as in Example 4, using A549-Dual cells were used and transfecting RNA-NP complexes. The RNA was the RIG-I agonist having SEQ ID NO: 2. The nanoparticle assayed were NP-1, variations of NP-1, where the solid inorganic metal particle (iron oxide for NP-1) was replaced with aluminum hydroxide, nanostructured lipid carrier (NLC) and a cationic nanoemulsion (CNE), where the NLC and CNE both lack an inorganic particle in a core region. Formulations had N:P ratios set to 8:1. The results, showed that the RNA RIG-I agonist stimulated IFN-β and IFIT2 production significantly better when complexed with any of the nanoparticle formulations having an inorganic core or with NLC compared to when complexed with CNE (data not shown). Of the nanoparticle formulations having an inorganic core, all upregulated IFIT2 production to roughly equivalent amounts, while complexing of iron oxide containing formulation (NP-1) yielded a slightly enhanced production of IFN-β (data not shown).

### Example 6: In-Vitro Stability of RIG-I Agonist Complexed to Nanoparticle Carrier

Various conditions were analyzed to assess biophysical characteristics of RIG-I agonist complexed to nanoparticle carriers stored at −80, −20, 4, 25, and 42 degrees Celsius over time. RNA integrity was assayed by gel electrophoresis and in vitro innate immune activation was assayed following storage under the same conditions.

Briefly, RIG-I agonist RNA encoding SEQ ID NO: 2 was complexed with NP-1 as described in previous examples above and stored at −80, −20, 4, 25, and 42 degrees Celsius. Samples were collected at 0, 1, 2, 4, 7, 14, and 28 days post preparation. Immediately after collection, particle size by Dynamic Light Scattering (DLS) and heterogeneity by Polydispersity index (PDI) was measured. Samples were aliquoted and stored in a −80 degrees Celsius freezer. From the samples, one aliquot was thawed, and RNA was also extracted from the samples and examined by RNA gel electrophoresis. Another aliquot was thawed, and transfected to A549-Dual cells, and SEAP and luciferase levels from the supernatant was observed as a measure of NF-κB and IFIT2 activation, respectively. RIG-I agonist RNA was prepared by diluting 38.8 μL RNA+611.2 μL water for a final concentration of 100 ng/μL. 640 μL of RIG-I agonist RNA was then complexed with 640 μL of NP-1 at an N:P ratio of 8, mixed by pipetting 10×. 200 μL aliquots were made in duplicate for each assay. After T=0 aliquots were taken, the tubes were placed into respective constant temperature incubators. Aliquots were taken from all tubes at each timepoint (T=0, 1, 2, 4, and 7 days post preparation). 30 μL aliquots were snap frozen in liquid nitrogen and then moved to minus 80 degrees Celsius storage.

For DLS measurements, 5 μL aliquots were taken at each time-point from samples and diluted into 995 μL water. PDI is a measure of the heterogeneity of a sample based on size. Particle size and PDI were measured using a Malvern Zetasizer Nano Label.

For cell transfections, 96-well plates were seeded overnight with A549-Dual cells (5e4 cells/well). Samples were thawed and five-point 2-fold dilution curves of each sample were prepared in a 96-well plate by diluting 12 μL of thawed complex into 288 μL 10% Sucrose/5 mM citrate buffer (top RNA concentration of 2 ng/μL) and performing 2-fold dilutions across the block. As a control, a fresh complex of RNA-NP-1 was prepared by diluting 2.4 μL RNA into 997.6 μL dH₂O at [4 ng/μL], and NP-1 was prepared by adding 10.7 μL of a 1/10 dilution of stock NP-1 to 989.3 μL 20% sucrose/citrate buffer, then 950 μL of RNA and NP-1 were mixed by pipetting 10× and incubated at RT for 30 min prior to serial dilution. On the day of transfection, growth media was removed from the plate by pipetting, 50 μL of Opti-MEM was added to the cells, 50 μL of each RNA:NP-1 dilution was added to cells, cells were incubated for 4 hours, RNA:NP-1 complex and Opti-MEM media were removed by pipetting and replaced with DMEM growth media and antibiotics appropriate to each cell line, and cells were incubated overnight. QUANTI-Blue™ and QUANTI-Luc™ (InvivoGen, San Diego, CA) were used to evaluate the NF-κB and IRF signaling pathways, respectively.

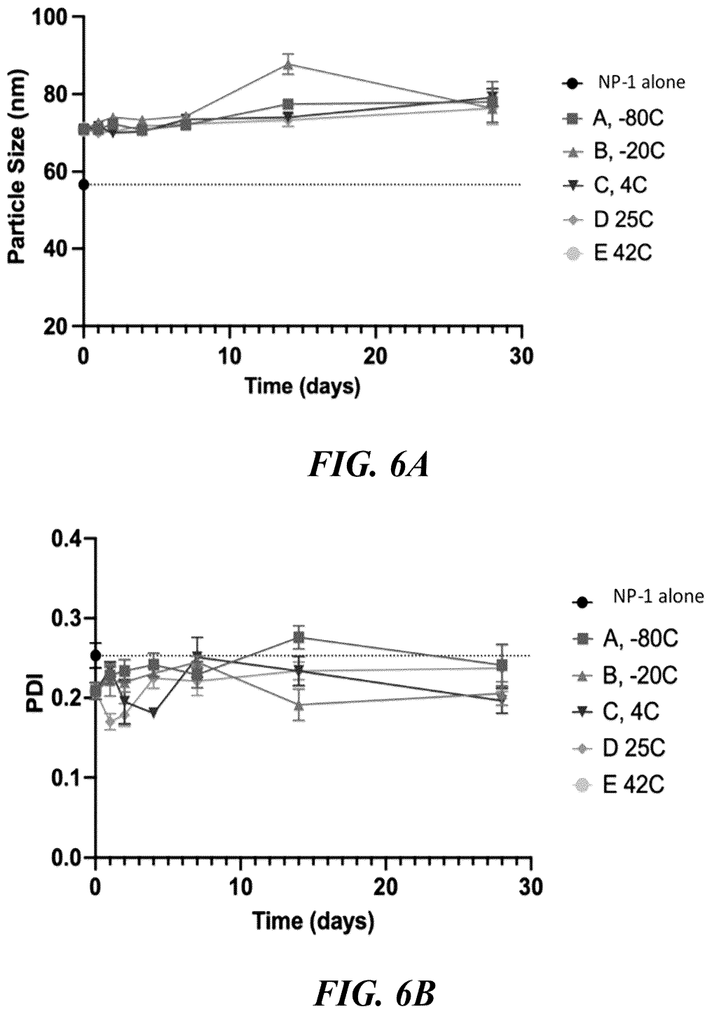

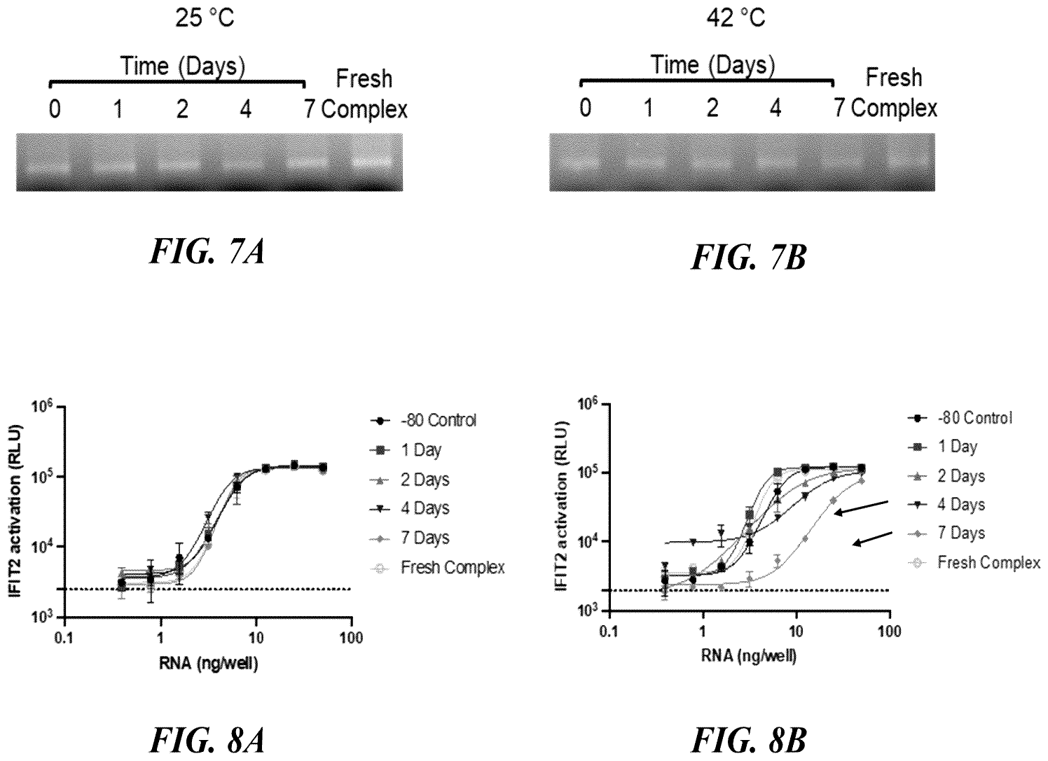

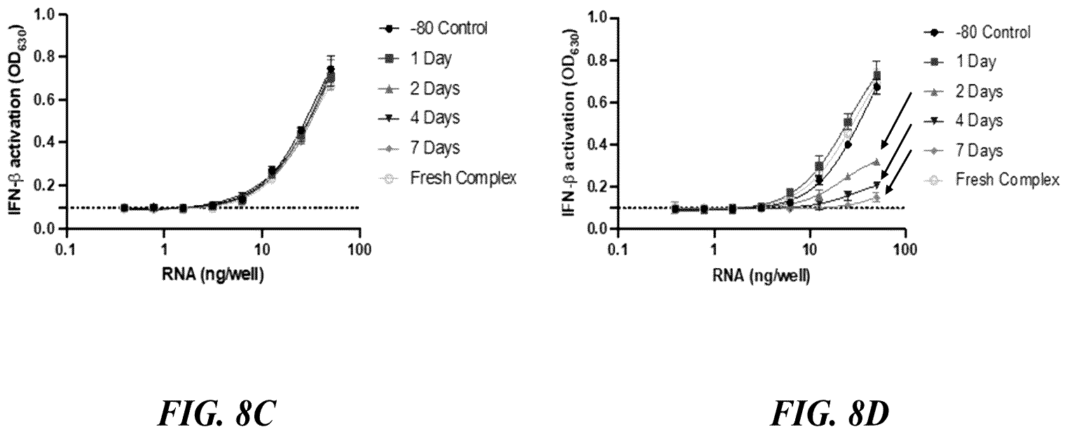

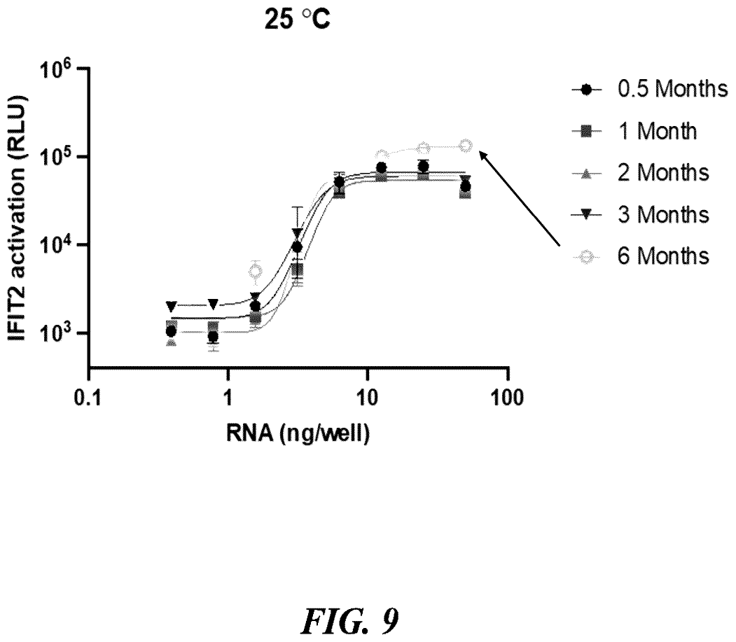

Biophysical analysis showed RIG-I agonist RNA having SEQ ID NO: 2 in complex with NP-1 having stability for up to 28 days when stored at −20, 4, 25, or 42 degrees Celsius with only minor increases in complex size over that time, as measured by particle size (FIG. 6A), and PDI (FIG. 6B). The complexes stored at up to up to 25 degrees Celsius maintained ability to activate expression of IFN-β in A549-Dual cells for at least 7 days (FIG. 8C), while losing about half of bioactivity in this assay by 4 days when held at 42 degrees Celsius (FIG. 8D). The complexes stored up to 42 degrees Celsius maintained ability to activate expression of IFIT2 in A549-Dual cells for at least 7 days (FIGS. 7B and 8B) and at 25 degrees Celsius (FIGS. 7A and 8A). Activation of IFIT2 activity over 0.5 to 6 months of RIG-I agonist RNA having SEQ ID NO: 2 in complex with NP-1 at N:P ratio of 8:1 was measured (FIG. 9). Compositions stored at −80 degrees Celsius, −20 degrees Celsius, and 4 degrees Celsius were also stable (data not shown).

### Example 7: Innate Immune Activation Assay

RIG-I dependence of various innate immune agonists was assessed with dose-response curves for (1) RIG-I RNA agonist having SEQ ID NO: 2 and (2) Riboxxim, a TLR3 agonist which is a dsRNA of SEQ ID NOS: 3-4 in A549-Dual cells. Briefly, A549-Dual cells were plated in a 96 well plate. Complexes of (1) RIG-I RNA agonist having SEQ ID NO: 2 and (2) Riboxxim were mixed with NP-1 having an N:P ratio of 8:1. Dilutions were prepared, cells were transfected with complexes 20 hours after plating in Opti-MEM for 4 hours followed by removal of media, replacement with DMEM overnight, and IFN-β and IFIT2 activation was measured using the Quanti-Blue and Quanti-Luc kits, respectively.

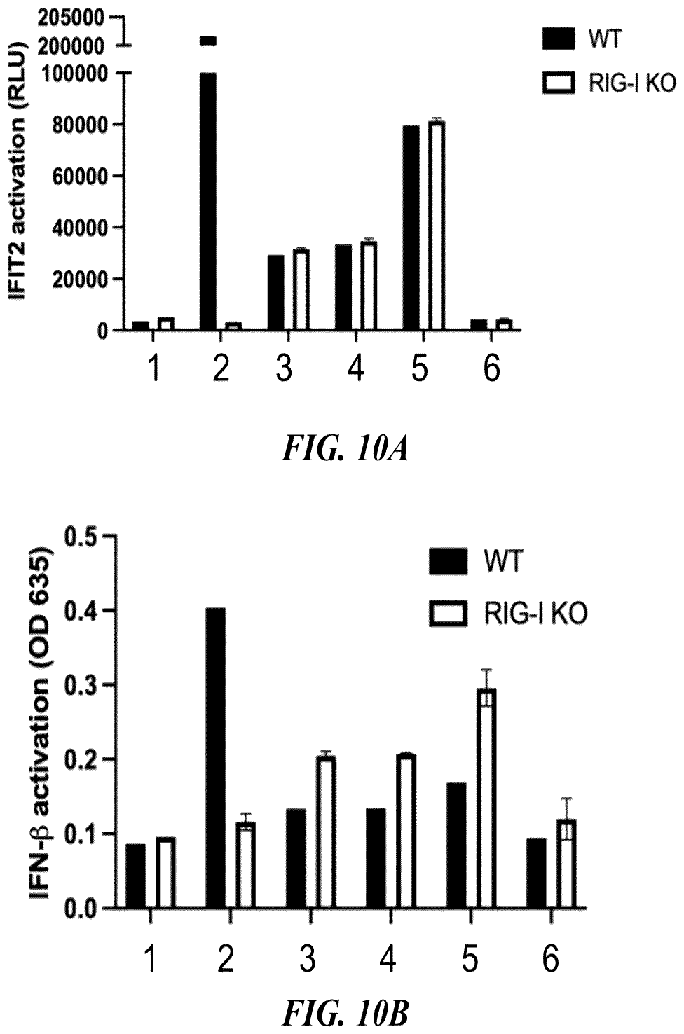

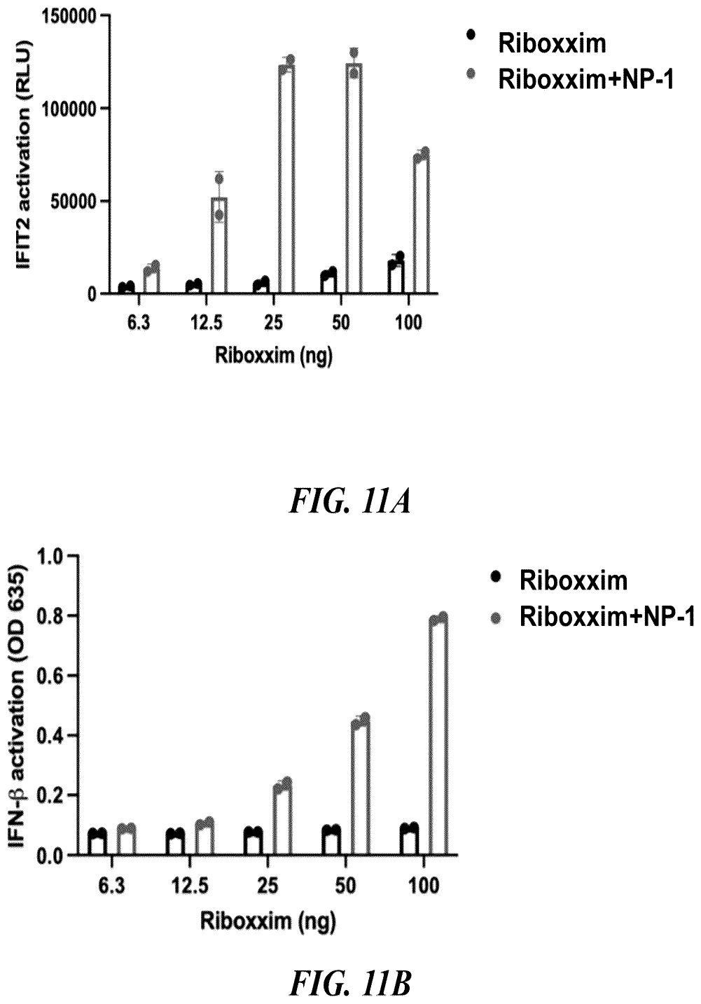

In a first assay, A549-Dual cells (WT and RIG-I KO) were treated under various conditions: (1) media, (2) RIG-I RNA agonist having SEQ ID NO: 2 complexed with NP-1; (3) IFN-α Leuk; (4) IFN-α Lymph; (5) IFN-β; or (6) TNF-α. Cell supernatants were monitored for IFIT2 (FIG. 10A) and IFN-β (FIG. 10B) activation by the presence of luciferase or SEAP in the supernatant, respectively. RIG-I RNA agonist having SEQ ID NO: 2 significantly upregulated IFIT2 and IFN-p production in a RIG-I-dependent manner. All other innate immune agonists assayed trigger IFIT2 activation even in the absence of RIG-I.

In a second assay, Riboxxim:NP-1 complexes were added to A549-Dual cells at the indicated Riboxxim doses (6.3, 12.5, 25, 50 and 100 ng). Cell supernatants were monitored for IFIT2 (FIG. 11A) and IFN-β (FIG. 11B) activation by the presence of luciferase or SEAP in the supernatant, respectively. Riboxxim stimulated IFIT2 and IFN-β production in a dose-dependent manner when complexed to NP-1, while Riboxxim alone had minimal activity.

### Example 8: Myeloid cell recruitment and activation assay

Study 1. Myeloid cell recruitment and activation was assayed in lymph nodes in response to complexed versus uncomplexed RIG-I RNA agonist having SEQ ID NO: 2 at 1 μg and 0.1 μg doses. Briefly, C57BL/6 mice were subcutaneously injected in the footpad with the following 4 groups: (1) untreated, (2) RIG-I RNA agonist having SEQ ID NO: 2 at 100 ng; (3) RIG-I RNA agonist having SEQ ID NO: 2 at 100 ng complexed to NP-1; (4) RIG-I RNA agonist having SEQ ID NO: 2 at 1 μg; (5) RIG-I RNA agonist having SEQ ID NO: 2 at 1 μg complexed to NP-1. 24 hours later, popliteal drained lymph node and spleen cells were collected for flow cytometry.

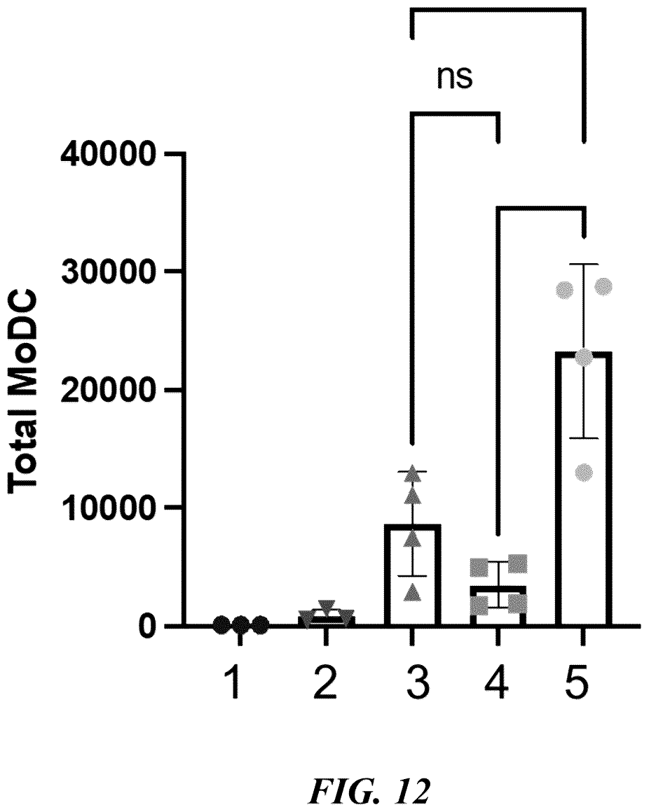

RIG-I RNA agonist/NP-1 complex induced robust inflammatory monocyte recruitment, even at a lower dose. The flow cytometry counts for CD11c+CD64+Ly6C+ inflammatory monocyte derived dendritic cells in the draining pLN Gated on CD3-B220-NK1.1-CD1ic+ single cells was collected (data not shown), and quantification of monocyte recruitment using the absolute cell count (FIG. 12: (1) untreated, (2) RIG-I RNA agonist having SEQ ID NO: 2 at 0.1 μg, (3) RIG-I RNA agonist having SEQ ID NO: 2 at 0.1 μg complexed with NP-1, (4) RIG-I RNA agonist having SEQ ID NO: 2 at 1 μg, and (5) RIG-I RNA agonist having SEQ ID NO: 2 at 1 μg complexed with NP-1).

RIG-I RNA agonist/NP-1 complex enhanced cDC1 responses. Flow cytometry plots (data not shown) and total quantification (FIG. 13A) were generated quantifying the frequency of XCR1+CD11b-LN-Resident cDC1s. Gated on CD3-B220-NK1.1-CD64-CD11c+ MHC-II int single cells. Flow cytometry plots (data not shown) and quantification of the frequency of CCR7+LN-resident cDC1s, indicating cell activation (FIG. 13B). Flow cytometry plots (not shown) and quantification (FIG. 13C) of the frequency of CD80+CD86++LN-resident cDC1s, indicating co-stimulatory molecule expression. Flow cytometry plots (data not shown) and quantification of the frequency of CCR7+ splenic dendritic cells (FIG. 13D), indicated cell activation. The results show that the RIG-I agonist:NP-1 complex enhanced inflammatory monocyte recruitment to lymph nodes relative to the RIG-I agonist alone. The robust resident DC activation indicated significant drainage of NP-1 to lymph nodes. The RIG-I agonist:NP-1 complex also activated cDC1s and increased their expression of co-stimulatory molecules. Systemic DC activation was observed with RIG-I agonist:NP-1 complex at the 1 μg dose.

Study 2. Myeloid cell recruitment and activation was assayed in drained lymph nodes in response to complexed versus uncomplexed RIG-I RNA agonist having SEQ ID NO: 2.

Briefly, C57BL/6 mice females 7 weeks old were subcutaneously injected in the footpad with the following 4 treatment conditions, in 20 μL volumes: (1) untreated; (2) R848 (a TLR7 and TLR8 agonist); (3) RIG-I RNA agonist having SEQ ID NO: 2 at 1 μg; (4) RIG-I RNA agonist having SEQ ID NO: 2 at 1 μg complexed to NP-1. 24 hours later, popliteal drained lymph node and spleen cells were collected for flow cytometry.

Mouse footpads were observed as more inflamed under RIG-I RNA agonist/NP-1 complex than RIG-I RNA agonist alone (data not shown). RIG-I RNA agonist/NP-1 complex showed increased inflammatory monocyte infiltration, increased frequency and preferential activation of Res cDC1s, and more CCR7+ and CD80+CD86+ Res cDCs compared to RIG-I RNA agonist alone. Specifically, flow cytometry analysis was done to assess recruitment of CD64+Ly6C+ inflammatory monocytes in the draining pLN, gated on CD3-B220-NK1.1-CD11c+ single cells. Quantification of monocyte recruitment, both as a percentage of total lymphocytes (FIG. 13E) or the absolute cell count (FIG. 13F). Flow cytometry plots (data not shown) and quantification collected to assess the frequency of XCR1+CD11b-LN-resident cDC1s (FIG. 13G), gated on CD3-B220-NK1.1-CD64-CD11c+ MHC-II int single cells. Flow cytometry plots (data not shown) and quantification were collected on the frequency of CCR7+LN-resident cDC1s, indicating cell activation (FIG. 13H). Flow cytometry plots (data not shown) and quantification of the frequency of CD80+CD86++LN-resident cDC1s were collected (FIG. 13I), indicating co-stimulatory molecule expression. Flow cytometry plots (data not shown) and quantification (FIG. 13J) of the frequency of CCR7+ splenic dendritic cells were generated, indicating limited systemic DC activation following RIG-I RNA agonist treatment.

### Example 9: Mouse Melanoma Cancer Model Systems

Female SCID mice were inoculated with 1×10⁵ B16FO (mouse melanoma) tumor cells in the right flank. Tumor development was monitored. On day 11, tumors were measured and mice were treated by intratumoral injection with either diluent alone (n=9), 100 ng RIG-I RNA agonist having SEQ ID NO: 2 in diluent (n=9), 100 ng RIG-I RNA agonist having SEQ ID NO: 2 formulated with NP-1 (n=8), or 100 ng RIG-I RNA agonist having SEQ ID NO: 2 formulated in a squalene emulsion (SE) (n=7). Mice in each cage were treated similarly, with injector and monitor unaware of treatment status. Results are presented as mean for each group (1-diluent; 2-RIG I agonist alone; 3-RIG I agonist-NP-1 complex; 4-RIG I agonist-SE complex). RIG I agonist-NP-1 together showed a comparative reduction in tumor growth (FIG. 14). Mice were terminated when tumor volume exceeded 1500 mm³, or rupture of lesion.

Neither unformulated RIG-I agonist nor RIG-I agonist-SE significantly reduced tumor size relative to intratumoral injection of RIG-I agonist in diluent. Tumors in RIG-I agonist-NP-1 complex-treated SCID mice were significantly smaller (days 14 and 15) than those in diluent-treated SCID mice. Survival of mice (removal was due to tumor size) was enhanced by RIG-I agonist-NP-1 complex treatment.

Female C57BL/6 mice were inoculated with 1×10⁵ B16F0 tumor cells in the right flank then tumor development monitored. On day 11, tumors were measured, and mice were treated by intratumoral injection with either diluent alone, 100 ng RIG-I RNA agonist having SEQ ID NO: 2 in diluent, 100 ng RIG-I RNA agonist having SEQ ID NO: 2 formulated with NP-1, or 100 ng RIG-I RNA agonist having SEQ ID NO: 2 formulated in SE. Mice in each cage were treated similarly, with injector and monitor unaware of treatment status. n=5 per group with the exception of RIG-I RNA agonist having SEQ ID NO: 2 alone (n=4). Results are presented as mean and SEM for each group. See FIG. 15. Mice were terminated when tumor volume exceeded 1500 mm³, or rupture of lesion. The 100 ng dose of RIG-I RNA agonist having SEQ ID NO: 2 in complex with NP-1 reduced tumor rate more than the other conditions. The NP-1 formulation improved therapeutic efficacy. Survival of mice (removal was due to tumor size) was enhanced by RIG-I agonist-NP-1 complex treatment.

### Multiple RIG-I Agonist Administrations.

Mice (C57BL/6, female, 6-8 weeks old) were inoculated with B16F0 tumor cells in the right flank. Melanoma growth was monitored with micrometer, allowing to reach approximately 150 mm³ (by day 10). Mice were then treated with intratumoral inoculation as per the condition table, Table 5. Briefly, on day 0, 1×10e5 B16F0 cells were inoculated into the flank of mice by subcutaneous injection, followed by treatment conditions on day 7, 10, and 14. Mice were either untreated (n=17), or treated once (×1) by peritumoral injection with 100 ng RIG-I RNA agonist having SEQ ID NO: 2/NP-1 (tumors were not observable at day 7; n=8), or treated three times (x 3) with 100 ng RIG-I RNA agonist having SEQ ID NO: 2/NP-1 initially by peritumoral injection (tumors were not observable at day 7) then by intratumoral injection on days 10 and 14; n=10).

TABLE 5

| | | RNA dose | Volume | |
|---|---|---|---|---|
| Group | Treatment | [μg] | [μl] | Route |
| 1 | None | — | 50 | — |
| 3 | 1 treatment: | 0.1 | 50 | Intratumoral |
| | RIG-I RNA agonist having | | | |
| | SEQ ID NO: 2/NP-1 | | | |
| 4 | 3 treatment: | 0.1 | 50 | Intratumoral |
| | RIG-I RNA agonist having | | | |
| | SEQ ID NO: 2/NP-1 | | | |

First terminations occurred on day 14, so reflects to that point as growth curves adjust as animals are removed. The three dose (and also two doses considering that dose three was provided on day 14) regimen conferred protection more apparent than a single dose regimen or no treatment measure by tumor volume (FIG. 16).

### Example 10: Intranasal Delivery of RIG-I Agonist and Nanoparticle Carrier

The following assays were conducted to assess (i) dose of RIG-I agonist on upregulation of innate immune genes in the lung, (ii) impact of N:P mass ratio on upregulation of innate immune genes in the lung, and (iii) impact on NP-1 on delivery of the RIG-I agonist.

Briefly, for each condition 8× C57BL/6 mice 79 days (11 weeks) old at initiation were subjected to Ketamine/Xylazine anesthesia on day 0. Mice were weighed and subjected to intranasal delivery. 8 hours later, mice were weighed, and lungs and nasal cavities were harvested from 3 mice stored in RNA later. Tissues were homogenized, and RNA was purified. qRT-PCR analysis was performed on select innate immune genes. Weight was monitored from 5 mice until weights stabilized, at 24 hours, 2 days and onward. The treatment groups were as summarized in Table 6. Intranasal delivery of unformulated, and NP-1 formulated, RIG-I RNA agonist having SEQ ID NO: 2 induced transient weight loss followed by recovery (FIG. 17).

As can be seen for IFN-β (FIG. 18A), delivery of 1 μg RIG-I RNA agonist having SEQ ID NO: 2 with NP-1 produced greater gene expression versus 1 μg RIG-I RNA agonist alone in the lung. This result was consistent in a similar assay, but with 5 ug RNA dose of RIG-I RNA agonist having SEQ ID NO: 2 with NP-1, versus an XRNA control and a transfection reagent control (FIG. 18B).

In a separate and similar assay, but where mice where not anesthetized, the 1 μg dose again showed greater gene expression for IFN-β in the lungs. However, in the nasal samples, the 5 μg dose of the RIG-I agonist having SEQ ID NO: 2 with NP-1 produced greater gene expression compared to a 5 μg dose (data not shown).

TABLE 6

| | | | RNA dose | NP-1 (DOTAP) | Volume | |
|---|---|---|---|---|---|---|
| Group | n | Treatment | [μg] | Conc. [μg] | [μl] | Route |
| 1 | 8 | Sucrose/Citrate buffer | — | — | 50 | Intranasal |
| 2 | 8 | RIG-I RNA agonist having | 5 | — | 50 | Intranasal |
| | | SEQ ID NO: 2 | | | | |
| 3 | 8 | RIG-I RNA agonist having | 1 | — | 50 | Intranasal |
| | | SEQ ID NO: 2 | | | | |
| 4 | 8 | RIG-I RNA agonist having | 0.2 | — | 50 | Intranasal |
| | | SEQ ID NO: 2 | | | | |
| 5 | 8 | RIG-I RNA agonist having | 5 | 160 | 50 | Intranasal |
| | | SEQ ID NO: 2/NP-1 | | | | |
| 6 | 8 | RIG-I RNA agonist having | 1 | 32 | 50 | Intranasal |
| | | SEQ ID NO: 2/NP-1 | | | | |
| 7 | 8 | RIG-I RNA agonist having | 0.2 | 6.4 | 50 | Intranasal |
| | | SEQ ID NO: 2/NP-1 | | | | |
| 8 | 8 | RIG-I RNA agonist having | 1 | 160 | 50 | Intranasal |
| | | SEQ ID NO: 2/NP-1 | | | | |
| 9 | 8 | RIG-I RNA agonist having | 0.2 | 160 | 50 | Intranasal |
| | | SEQ ID NO: 2/NP-1 | | | | |

### Example 11: Pre-Treatment Against SARS-CoV-2 Challenge in Mice

RIG-I agonist was assessed as a pretreatment in a SARS-CoV-2 animal model of disease. Briefly, on day zero, C57BL/6 female mice 10 weeks old were treated under treatment conditions described below (Table 7) with intranasal delivery of the treatment condition, anesthetized with ketamine/xylazine, and weighed. A day later, mice were weighed and subjected to intranasal delivery of the infection agent (mouse-adapted SARS-CoV-2 (COV2.MA10)) under ketamine/xylazine anesthesia. On days 2-8, mice were weighed daily and monitored for morbidity.

TABLE 7

| | | | RNA dose | N:P | | Volume |
|---|---|---|---|---|---|---|
| Group | n | Treatment | [μg] | Ratio | Infection | [μl] |
| 1 | 10 | Sucrose/Citrate buffer | — | — | Mock | 50 |
| 2 | 10 | RIG-I RNA agonist having | 5 | 8 | Mock | 50 |
| | | SEQ ID NO: 2/NP-1 | | | | |
| 3 | 10 | XRNA: NP-1 | 5 | 8 | Mock | 50 |
| 4 | 10 | Sucrose/Citrate buffer | 0 | — | CoV2.MA10 | 50 |
| 5 | 10 | RIG-I RNA agonist having | 0.2 | 8 | CoV2.MA10 | 50 |
| | | SEQ ID NO: 2/NP-1 | | | | |
| 6 | 10 | RIG-I RNA agonist having | 1 | 8 | CoV2.MA10 | 50 |
| | | SEQ ID NO: 2/NP-1 | | | | |
| 7 | 10 | RIG-I RNA agonist having | 5 | 8 | CoV2.MA10 | 50 |
| | | SEQ ID NO: 2/NP-1 | | | | |
| 8 | 10 | XRNA: NP-1 | 5 | 8 | CoV2.MA10 | 50 |

Weights of control mice pre-treated with 5 μg of RIG-I RNA agonist having SEQ ID NO: 2 or XRNA or with sucrose/citrate buffer are shown in FIG. 19A. Weights of mice treated with 5, 1, or 0.2 μg of RIG-I RNA agonist having SEQ ID NO: 2:NP-1 and challenged with 10⁴ PFU CoV2 MA10 compared to control mice are shown in FIG. 19B. (C) Weights of mice treated with 5 μg XRNA:NP-1 and challenged with 10⁴ PFU CoV2.MA10 compared to control mice are shown in FIG. 19C. Comparison of weighs of mice treated with 0.2 μg RIG-I RNA agonist having SEQ ID NO: 2:NP-1 compared to control mice as shown in FIG. 19D. The RIG-I agonist:NP-1 treatment prevented weight-loss associated with CoV2.MA10 infection down to 0.2 μg of RIG-I agonist. RIG-I agonist:NP-1 showed a dose-dependent weight loss when delivered in high doses via the intranasal route. 5 μg XRNA:NP-1 also showed weight loss when delivered via the intranasal route and protected mice from CoV2.MA10-associated weight loss.

### Example 12: Pre-Treatment Against SARS-CoV-2 Challenge in hACE-Transgenic Mice

RIG-I agonist was assessed as a pretreatment in a human ACE (hACE) transgenic mouse model of SARS-CoV-2 infection. Briefly, on day zero, hACE2 C57BL/6 female mice 10 weeks old were treated under treatment conditions described below (Table 8) with intranasal delivery of the treatment condition, anesthetized with ketamine/xylazine, and weighed. A day later, mice were weighed and subjected to intranasal delivery of the infection agent (2019-nCoV/USA-WA1-2020) under ketamine/xylazine anesthesia. On days 2-7, mice were weighed daily and monitored for morbidity.

TABLE 8

| | | | RNA | N:P | | Volume | |
|---|---|---|---|---|---|---|---|
| Group | n | Treatment | dose [μg] | Ratio | Infection | [μl] | Route |
| 1 | 10 | Sucrose/Citrate buffer | — | — | Mock | 50 | Intranasal |
| 2 | 10 | RIG-I RNA agonist having | 1 | 8 | Mock | 50 | Intranasal |
| | | SEQ ID NO: 2/NP-1 | | | | | |
| 3 | 10 | Sucrose/Citrate buffer | — | — | SARS-CoV-2 | 50 | Intranasal |
| 4 | 10 | RIG-I RNA agonist having | 1 | 8 | SARS-CoV-2 | 50 | Intranasal |
| | | SEQ ID NO: 2/NP-1 | | | | | |

Anesthetized mice were pre-treated with the indicated dose of RIG-I agonist:NP-1 or mock-treated with a sucrose/citrate buffer. After 24 hours, mice were anesthetized again and either mock-challenged or challenged with SARS-CoV-2 (2019-nCoV/USA-WA1/2020). Weights were monitored over 7 days. RIG-I agonist:NP-1 treatment prevented weight-loss associated with SARS-CoV-2 infection in hACE2 mice, and did not cause any weight loss above mock treatment (FIG. 20).

### Example 13: Nanoparticle Delivery of DNA

The assay assessed delivery of various nanoparticles having DNA or RNA admixed therewith. Briefly, DNA encoding secreted embryonic alkaline phosphatase (SEAP) or replicon RNA encoding an RNA polyerase and SEAP were prepared and mixed with a nanoparticle of NP-1 or NP-3. Conditions are provided in Table 9 BALB/c female mice were injected intramuscularly (IM). Nucleic acid preparations for dilutions are provided in Table 10. Nanoparticle preparations are provided in Table 11. Nucleic acid-nanoparticle complexes were formed by adding 150 μl diluted NP-1 or NP-3 to 150 μl diluted DNA or RNA, then incubated for at least 30 minutes.

TABLE 9

| | | | | | | | Inj. | |
|---|---|---|---|---|---|---|---|---|
| | | For- | | RNA | DNA | | Vol- | |
| | | mula- | DNA/RNA- | dose | dose | | ume | |
| Group | N | tion | SEAP | [μg] | [μg] | N:P | [μl] | Route |
| 1 | 5 | Naked | DNA-SEAP | | 20 | n/a | 50 | IM |
| 2 | 5 | NP-1 | DNA-SEAP | | 10 | 15 | 50 | IM |
| 3 | 5 | NP-1 | DNA-SEAP | | 10 | 7.5 | | IM |
| 4 | 5 | NP-1 | DNA-SEAP | | 20 | 15 | | IM |
| 5 | 5 | NP-1 | DNA-SEAP | | 20 | 7.5 | | IM |
| 6 | 5 | NP-1 | RNA-SEAP | 1 | | 15 | 50 | IM |
| 7 | 5 | NP-3 | RNA-SEAP | 1 | | | 50 | IM |

TABLE 10

| | | | 40% | | | Concentrations | |
|---|---|---|---|---|---|---|---|
| | | DNA or | su- | | | measure prior | |
| | DNA- or | RNA | crose | water | Total | to complexing | |
| Group | RNA-SEAP | [μl] | [μl] | [μl] | [μl] | using NanoDrop | |
| 1 | DNA-SEAP | 24.0 | 75.0 | 51.0 | 150.0 | 725 | ug/ml |
| 2 | DNA-SEAP | 12.0 | 0.0 | 138.0 | 150.0 | 528 | ug/ml |
| 3 | DNA-SEAP | 12.0 | 0.0 | 138.0 | 150.0 | 528 | ug/ml |
| 4 | DNA-SEAP | 24.0 | 75.0 | 51.0 | 150.0 | 725 | ug/ml |
| 5 | DNA-SEAP | 24.0 | 75.0 | 51.0 | 150.0 | 725 | ug/ml |
| 6 | RNA-SEAP | 2.7 | 0.0 | 147.3 | 150.0 | 57 | ug/ml |
| 7 | RNA-SEAP | 2.7 | 0.0 | 147.3 | 150.0 | 57 | ug/ml |

TABLE 11

| | | | 40% | 100 mM | | |
|---|---|---|---|---|---|---|
| | | NP-1 | sucrose | citrate | Water | Total |
| Group | Formulation | [μl] | [μl] | [μl] | [μl] | [μl] |
| 1 | Naked | 0 | 0 | 15 | 135 | 150 |
| 2 | 100-015 | 72 | 90 | 18 | 0 | 180 |
| 3 | 100-015 | 36 | 90 | 18 | 36 | 180 |
| 4 | 100-015 | 144 | 0 | 18 | 18 | 180 |
| 5 | 100-015 | 72 | 0 | 18 | 90 | 180 |
| 6 | 100-015 | 7.2 | 90 | 18 | 64.8 | 180 |
| | | | | | | 180 |

Mice were inoculated on day 0 according to the treatment groups. Blood was collected on days 4, 6 and 8, allowed to clot, and the serum was collected and stored at minus 80 degrees Celsius. Serum samples were thawed, and SLAP detection was assessed. A chemiluminescent substrate of SLAP was provided, and activity was measured based on the light generated, and quantitated as Relative Luminescence Units (RLUs). Results are shown in FIGS. 21A-21C and FIGS. 22A-22C, with a mean, n=5 per group. NP-1 and NP-3 formulations enhanced target protein production over delivery of DNA alone. Inclusion of Miglyol in NP-3 enhanced protein production of RNA over standard NP-1 having squalene at Day 4.

### Example 14: Additional Nanoparticle Formulations

Additional nanoparticle formulations are produced according to the following tables (Table 12 and Table 13).

TABLE 12
RNA Formulation.

| | Solution for | | | |
|---|---|---|---|---|
| Dosage form: | Injection Each 0.5 ml | | | Concentration |
| Composition: | Vial Contains: | Quantity | | (mg/ml) |
| | RNA | 25 | mcg | 0.05 |
| | DOTAP | 0.75 | mg | 1.5 |
| | Iron Oxide Nanoparticles | 0.005 | mg | 0.01 |
| | Squalene | 0.94 | mg | 1.88 |
| | Sorbitan Monostearate | 0.93 | mg | 1.86 |
| | Polysorbate 80 | 0.93 | mg | 1.86 |
| | Sucrose IP | 50 | mg | 100 |
| | Citric Acid Monohydrate | 1.05 | mg | 2.1 |
| | Water for Injection | q.s. to 0.5 | ml | |

TABLE 13
Lyophilized RNA Formulation.

| | Lyophilized powder | | | Ap- |
|---|---|---|---|---|
| | Each 5 | | Con- | proximate |
| Dosage form: | dose vial | | centration | dry |
| Composition: | contains: | Quantity | (mg/ml) | weight % |
| | RNA | 50 mcg | 0.02 | 0.02 |
| | DOTAP | 1.5 mg | 0.6 | 0.57 |
| | Squalene | 1.88 mg | 0.752 | 0.72 |
| | Sorbitan | 1.86 mg | 0.744 | 0.71 |
| | Monostearate | | | |
| | Polysorbate 80 | 1.86 mg | 0.744 | 0.71 |
| | Sucrose IP | 250 mg | 100 | 95.3 |
| | Citric Acid | 5.25 mg | 2.1 | 2 |
| | Monohydrate | | | |
| | Water for | 2.5 ml | | |
| | Injection (for | | | |
| | reconstitution) | | | |

### Example 15: Evaluation of Lyophilized Vaccines in Mice

The following was performed to assay activity of lyophilized NP-1 with replicon RNA encoded SARS-CoV-2 spike antigen sequence, physicochemical properties of reconstituted vaccines, potency, and immunogenicity. Briefly, materials in Table 14 were used.

The following was performed to assay activity of lyophilized NP-1 with replicon RNA encoded SARS-CoV-2 spike antigen sequence, physicochemical properties of reconstituted vaccines, potency, and immunogenicity. Briefly, materials in Table 14 were used.

TABLE 14
Materials.

| Name | Stock concentration |
|---|---|
| NP-1 | 30 mg/ml |
| | (measuring DOTAP conc.) |
| NP-7 | 30 mg/ml |
| | (measuring DOTAP conc.) |
| repRNA-CoV2-spike (wild type) | 1687 μg/ml |
| VEE-S-v5 Delta (“WT-S”) | |
| repRNA-CoV2-spike (delta) | 783 μg/ml |
| VEE-nCoV19-S-Delta.AY1- | |
| S2P-wtFur (“Delta-S”) | |
| Sucrose (EMD, Millipore) | — |
| Na-citrate (Teknova) | 1 M |

Preparation of formulation complexes. Compositions of lipid nanoparticle/RNA complexes were prepared in this assay as shown below in Table 8. NP-1 or NP-7 and repRNAs were complexed at a N-to-P ratio of 15 and complexed to obtain a final repRNA concentration of 50 mg/ml or 100 mg/ml (“2×” material), and μm or 20% w/v sucrose content, respectively. Complexed material with 100 sucrose (50 mg/ml repRNA) contained 5 mM sodium citrate while that with 20 sucrose (100 mg/ml repRNA) contained 10 mM citrate. Complexes were filled in 2 ml sterile, depyrogenated and baked vials. Complexes with 10% sucrose were filled at 0.7 ml per vial and 20% sucrose at 0.35 ml per vial. Vials were then either lyophilized and stored or stored as is in liquid form. Storage temperature was 25 degrees Celsius or 42 degrees Celsius for 1 week. Quantity of lyophilized and liquid vials per composition is summarized in Table 15.

TABLE 15
Formulations and Characteristics.

| | | DOTAP | RNA | Volume per | Lyo | Liquid |
|---|---|---|---|---|---|---|
| Description | N:P | [μg/ml] | [μg/ml] | vial [ml] | vials | vials |
| NP-1 + WT-S | 15 | 1500 | 50 | 0.7 | 8 | 6 |
| in 10% sucrose | | | | | | |
| NP-1 + Delta-S | 15 | 1500 | 50 | 0.7 | 2 | 0 |
| in 10% sucrose | | | | | | |
| NP-1 + WT-S | 15 | 3000 | 100 | 0.35 | 8 | 0 |
| in 20% sucrose | | | | | | |
| NP-1 + Delta-S | 15 | 3000 | 100 | 0.35 | 2 | 0 |
| in 20% sucrose | | | | | | |
| NP-7 + WT-S | 15 | 1500 | 50 | 0.7 | 8 | 6 |
| in 10% sucrose | | | | | | |

Lyophilization cycle. An SP VirTis Advantage Pro tray and batch lyophilizer with inert gas fill and stoppering capability was used. Summary of the lyophilization cycle is shown in Table 16 below. After end of cycle, vials were backfilled with nitrogen at 48 torr and stoppered, before equilibrating to room pressure.

TABLE 16
Conditions.

| Time | Temp | Pressure | |
|---|---|---|---|
| [hours] | [° C.] | [mT] | Notes |
| 0 | 5 | 760 | Shelf pre-cooled to 5 degrees C |
| 0.5 | 5 | 760 | Freezing |
| 2 | −50 | 760 | |
| 2.5 | −50 | 50 | Evacuation |
| 3 | −30 | 50 | Primary drying |
| 20.5 | −30 | 50 | |
| 22.5 | 25 | 50 | Secondary drying |
| 24 | 25 | 50 | |

Condition groups. A summary of 14 groups analyzed in this assay is provided in Table 17 below. Groups 1 and 4, as indicated in the storage column, were prepared fresh to serve as positive controls for comparison with standard protocol for vaccine preparation.

TABLE 17
Conditions.

| | | | Sucrose | | Storage |
|---|---|---|---|---|---|
| Group | Formulation | RNA | [% w/v] | Form | [temp/time] |
| 1 | NP-1 | WT-S | 10 | Liquid | Fresh |
| 2 | NP-1 | WT-S | 10 | Liquid | 25° C./1 wk |
| 3 | NP-1 | WT-S | 10 | Liquid | 42° C./1 wk |
| 4 | NP-7 | WT-S | 10 | Liquid | Fresh |
| 5 | NP-7 | WT-S | 10 | Liquid | 25° C./1 wk |
| 6 | NP-7 | WT-S | 10 | Liquid | 42° C./1 wk |
| 7 | NP-1 | WT-S | 10 | Lyo | 25° C./1 wk |
| 8 | NP-1 | WT-S | 10 | Lyo | 42° C./1 wk |
| 9 | NP-1 | WT-S | 20 | Lyo | 25° C./1 wk |
| 10 | NP-1 | WT-S | 20 | Lyo | 42° C./1 wk |
| 11 | NP-7 | WT-S | 10 | Lyo | 25° C./1 wk |
| 12 | NP-7 | WT-S | 10 | Lyo | 42° C./1 wk |
| 13 | NP-1 | Delta-S | 10 | Lyo | 25° C./1 wk |
| 14 | NP-1 | Delta-S | 20 | Lyo | 25° C./1 wk |

Immunogenicity assay. Induction of anti-spike IgG responses were evaluated in 6 to 8 weeks old female C57B31/6 mice. A group size of 5 mice was used. The schedule is shown in Table 18.

TABLE 18
Immunogenicity Schedule.

| Date | Day | Procedure |
|---|---|---|
| Aug. 23, 21 | −7 | Lyophilization |
| Sept. 01, 21 | 0 | Immunization by IM route |
| Sept. 15, 21 | 14 | Bleed |
| Sept. 29, 21 | 28 | Bleed |
| Oct. 8, 21 | 37 | Mice sacrificed |

After 1 week of storage in 25 degrees Celsius or 42 degrees Celsius stability chamber, lyophilized nanoparticle/RNA complexes were reconstituted in 0.7 ml sterile milliQ water and gently swirled until no particles were visible to the naked eye. Particle size (z-average) and size distribution (PDI) of the complexes was measured and is summarized in FIG. 23, with group designations shown in Table 19. Particle size and PDI of freshly prepared NP-1/WT-S complex (group 1) was 76.8 nm and 0.223, respectively. After reconstitution, lyophilized samples (groups 7-14) grew by an average of 45% (+/−11%). Summary of % change in z-average relative to group 1 is included in Table 19.

TABLE 19
Percent % Change in Z-Average.

| Group # | 2 | 3 | 4 | 5 | 6 | 7 | 8 | 9 | 10 | 11 | 12 | 13 | 14 |
|---|---|---|---|---|---|---|---|---|---|---|---|---|---|
| % change | 2% | 0% | 15% | −4% | −1% | 30% | 42% | 59% | 53% | 48% | 33% | 41% | 55% |
| z-average | | | | | | | | | | | | | |
| vs. group 1 | | | | | | | | | | | | | |

### Example 16: SEQ ID NO: 2 and NP-1 and Immune Response

NP-1 nanoemulsions were generated as described above and complexed with SEQ ID NO: 2. Female C57BL/6 mice were inoculated with 1×10⁵ B16FO tumor cells in the flank then tumor development monitored. Mice were either treated with diluent or treated once by peritumoral injection with 0.1 μg or 1 μg SEQ ID NO: 2 RNA formulated with NP-1 on day 12 (SEQ ID NO: 2+NP-1). Mice were individually marked, and coded treatment provided in a cage-based manner to disrupt awareness of treatment status. Tumor draining lymph nodes were collected on experiment day 13, single cell suspensions prepared and subjected to flow cytometry to determine the cell phenotypes indicated.

Results: FIGS. 24A-24B shows intratumoral CD4+ T cell number and intratumoral CD4+ T cell number as a function of tumor volume. FIGS. 25A-25B shows intratumoral CD8+ T cell number and intratumoral CD8+ T cell number as a function of tumor volume. FIGS. 26A-26B shows intratumoral monocyte-derived dendritic cell (MoDC) number and intratumoral monocyte-derived dendritic cell (MoDC) number as a function of tumor volume. FIGS. 27A-27B shows conventional dendritic cell (cDC) number and intratumoral cDC number as a function of tumor volume. FIG. 27C shows a graph of CD86 expression in cDCs. FIGS. 28A-28B shows intratumoral tumor-associated macrophage (TAM) number and intratumoral tumor-associated macrophage (TAM) number as a function of tumor volume. Y-axis: Cell number/tumor volume. X-axis: Condition. FIGS. 29A-29B show myeloid cell activation in tumor-draining lymph nodes (tdLNs) and CCR7 expression. FIGS. 30A-30C show CD4 and CD8 T cells isolated from tumor draining lymph nodes expressed interferon and T-box expressed in T cells (T-bet) expression. Animals treated with SEQ ID NO: 2 and NP-1 exhibited an increase in interferon-expressing CD4 T cells, an increase in T-bet expressing CD4+ and CD8+ T cells in the tdLN as compared to untreated animals. Within tumors animals treated with SEQ ID NO: 2 and NP-1 exhibited an increase in interferon expression and CD4+ T cell number/tumor volume in the tumor infiltrate as compared to untreated animals (FIGS. 31A-31B). Furthermore, the SEQ ID NO: 2+NP-1 significantly reduced tumor volume in treated animals as compared to animals treated with SEQ ID NO: 2 alone (FIG. 32).

Summary of Results: Enhanced recruitment of monocyte dendritic cells (MoDCs) to tumors with high dose SEQ ID NO: 2 and SEQ ID NO: 2 complexed to NP-1 as compared to control was observed. SEQ ID NO: 2 RNA alone was able to induce elevated recruitment of tumor-associated macrophages (TAMs) and enhanced dendritic cell infiltration of tumors. Elevated levels of intratumoral CD4+ and CD8+ T cells was also observed. SEQ ID NO: 2+NP-1 recruited monocyte-derived dendritic cells (MoDCs) to tumor-draining lymph nodes (tdLNs) and activates lymph node-resident dendritic cells better than RNA alone (in this case SEQ ID NO: 2).

### Example 17: Intranasal Administration of SEQ ID NO: 2 Complexed with NP-1 and Immune Stimulation Kinetics

NP-I was generated and complexed with SEQ ID NO: 2 as described above. Female C57BL/6 mice were anesthetized by ketamine/xylazine. SEQ ID NO: 2 and SEQ ID NO: 2 complexed with NP-1 at an N:P ratio of 8 were delivered intranasally (30 μL) or by footpad injection. Lungs, nasal cavities, and spleens harvested at 4, 8, 12, 24 hours. Innate immune activation was assessed by qRT-PCR. Cell activation kinetics of monocyte recruitment to the draining lymph nodes were also assessed. For comparison, footpad injections in mice were performed using control RNA, SEQ ID NO: 2 alone, and SEQ ID NO: 2+NP-1 (FIGS. 33A-33B).

Next, the expression of innate immune cell markers was measured in the lung, including CXCL10, IFIT1, IFIT2, and IFN-β (FIGS. 34A-34D). SEQ ID NO: 2+NP-1 increased all innate immune cell markers in the lungs of intranasally treated animals within 8 hours as compared to untreated controls and animals administered SEQ ID NO: 2 alone.

The expression of innate immune cell markers was assessed in the nasal cavity, including CXCL10, IFIT1, IFIT2, and IFN-β (FIGS. 35A-35D). SEQ ID NO: 2+NP-1 increased all innate immune cell markers in the nasal cavity of intranasally treated animals within 8 hours as compared to untreated controls and animals administered SEQ ID NO: 2 alone.

SEQ ID NO: 2 activated innate immune genes with peak upregulation at 12 hours post-intranasal delivery. Interestingly, the SEQ ID NO: 2+NP-1 complex enhances the kinetics of RNA-mediated innate immune activation, such that peak innate response is at 8 hours.

### Example 17: SEQ ID NO: 2 Delivery by Various Nanoparticle Formulations

Various nanoparticle compositions were generated and complexed with SEQ ID NO: 2 as described above at N:P ratio of 8. Table 19 provides a list of the various conditions.

### Basic Experimental Description:

A549-Dual cells were transfected with different nanoparticle and RNA complexes, including: NP-1, modified formulations of NP-1, cationic nanoemulsions (CNEs), and solid lipid nanoparticles (SLNs).

TABLE 19
Conditions.

| Description | Concentration | Location |
|---|---|---|
| NP-1 − squalene + 5 nm Fe core | 30 mg/mL DOTAP | +4° C., cold room |
| NP-1 emulsion − squalene | 30 mg/mL DOTAP | +4° C., cold room |
| SLN − solanesol | 30 mg/mL DOTAP | +4° C., cold room |
| SLN − dynasan | 30 mg/mL DOTAP | +4° C., cold room |
| NP-1 − miglyol + Fe core | 30 mg/mL DOTAP | +4° C., cold room |
| SLN − retinyl acetate | 15 mg/mL DOTAP | +4° C., cold room |
| SLN − trilaurin | 15 mg/mL DOTAP | +4° C., cold room |
| SLN − 1, 2, 3-tripalmitoyl glycerol | 15 mg/mL DOTAP | +4° C., cold room |
| NP-1 emulsion − “IV formulation” | 15 mg/mL DOTAP | +4° C., cold room |
| with PEG lipids on surface | | |
| CNE | 0.4 mg/mL DOTAP | +4° C., cold room |
| NP-1 emulsion − 2/3 squalene | 30 mg/mL DOTAP | +4° C., cold room |
| amount | | |
| NP-1 emulsion − 1/3 squalene | 30 mg/mL DOTAP | +4° C., cold room |
| amount | | |
| NP-1 emulsion − no squalene | 30 mg/mL DOTAP | +4° C., cold room |
| NP-1 emulsion − SPAN ® 80/Tween | 30 mg/mL DOTAP | +4° C., cold room |
| 60 | | |
| NP-1 − squalene + 15 nm Fe core | 30 mg/mL DOTAP | +4° C., cold room |
| SEQ ID NO: 2 | 1000 mg/mL | −80° C., freezer |

A549-Dual cells were seeded in a 96-well plate and incubate at 37 degrees C. +5% CO2 overnight. SEQ ID NO: 2 was diluted and formulations were generated. Cell were transfected in Opti-MEM for 4 hours. Media was removed and replaced with complete DMEM overnight. SEAP and luciferase levels were analyzed in the supernatant as a measure of IFN-β and IFIT2 activation, respectively. Innate immune activation markers were measured using the Quanti-Blue and Quanti-Luc kits respectively. All complexes stimulated IFN-β and IFIT2 activation, to varying degrees (FIGS. 36A-36B). An intravenous nanoparticle formulation containing PEG lipids on the surface and CNE both activated A549-Dual cells, but to significantly reduced levels. The standard nanoparticle formulation (NP-1) with 5 nm Fe particles; NP-1 modified with 15 nm Fe particles; a modified NP-1 emulsion with no Fe particles, had equivalent bioactivity for both IFN-β and IFIT2 activation (FIGS. 37A-37B). Nanoemulsions with different solid lipid nanoparticles successfully delivered SEQ ID NO: 2 to cells to upregulate both IFN-β and IFIT2 (FIGS. 38A-38B). A 4-fold difference was observed between different solid lipid nanoparticles with dynasan-containing nanoparticles significantly stimulating IFIT2 activation (FIG. 38B).

Next, serum interferon levels in 7-11 week old C57BL/6 mice were evaluated 14 hours after intramuscular injection of PAMP (SEQ ID NO: 2) complexed with 3 different lipid nanoparticles (LNPs) (FIG. 39). FIG. 39 shows that LNPs-formulated PAMPs can induce in vivo interferon alpha 2 (IFNα2) activity.

All cationic nanoparticle formulations tested delivered the RIG-I agonist (in this case a nucleic acid encoding SEQ ID NO: 2) to cells to activate markers of the innate immune response (IFN-β and IFIT2) in the A549-Dual reporter cell line. Furthermore, solid lipid nanoparticles (in this case solid dynasan core) also potentiated the expression of innate immune system markers.

While preferred embodiments of the present invention have been shown and provided herein, it will be obvious to those skilled in the art that such embodiments are provided by way of example only. Numerous variations, changes, and substitutions will now occur to those skilled in the art without departing from the invention. It should be understood that various alternatives to the embodiments of the invention provided herein may be employed in practicing the invention. It is intended that the following claims define the scope of the invention and that methods and structures within the scope of these claims and their equivalents be covered thereby.

What is claimed is:

1. A composition, wherein the composition comprises: a nanoparticle, wherein the nanoparticle comprises: a hydrophobic core, wherein lipids present in the hydrophobic core are in liquid phase at 25 degrees Celsius; and a hydrophilic surface; and a nucleic acid, wherein the nucleic acid comprises a region encoding a sequence that is at least 85% identical to any one of SEQ ID NOS: 1-11.

2. The composition of claim 1, wherein the nucleic acid comprises a double-stranded RNA.

3. The composition of claim 1, wherein the nucleic acid comprises a single-stranded RNA.

4. The composition of claim 1, wherein the sequence is at least 90% identical to SEQ ID NO: 1.

5. The composition of claim 1, wherein the sequence is at least 95% identical to SEQ ID NO: 1.

6. The composition of claim 1, wherein the sequence is SEQ ID NO: 1.

7. The composition of claim 1, wherein the sequence is at least 90% identical to SEQ ID NO: 2.

8. The composition of claim 1, wherein the sequence is at least 95% identical to SEQ ID NO: 2.

9. The composition of claim 1, wherein the sequence is SEQ ID NO: 2.

10. The composition of claim 1, wherein the sequence is at least 90% identical to SEQ ID NO: 3.

11. The composition of claim 1, wherein the sequence is at least 95% identical to SEQ ID NO: 3.

12. The composition of claim 1, wherein the sequence is SEQ ID NO: 3.

13. The composition of claim 1, wherein the nanoparticle is characterized as having a z-average diameter particle size measurement of up to 100 nm in diameter when measured by dynamic light scattering.

14. The composition of claim 1, wherein the nucleic acid is complexed to the nanoparticle.

15. The composition of claim 1, wherein the hydrophilic surface further comprises a cationic lipid.

16. The composition of claim 15, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount.

17. The composition of claim 15, wherein the cationic lipid is 1,2-dioleoyloxy-3 (trimethylammonium)propane (DOTAP), 3β-[N (N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)— heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3 0-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)— tetrakis (octadeca-9,12-dienoate); PEG2000-DMG, (R)-2,3-bis(myristoyloxy)propyl-1-(methoxy poly(ethylene glycol)2000) carbamate; or TT3, N1,N3,N5-tris(3-(didodecylamino)propyl)benzene-1,3,5-tricarboxamide.

18. The composition of claim 1, wherein the hydrophobic core comprises an oil.

19. The composition of claim 18, wherein the oil is α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E.

20. The composition of claim 19, wherein the triglyceride is capric triglyceride, caprylic triglyceride, a caprylic and capric triglyceride, a triglyceride ester, or myristic acid triglycerin.

21. The composition of claim 1, wherein the nanoparticle further comprises an inorganic particle.

22. The composition of claim 21, wherein the inorganic particle comprises a metal salt, a metal oxide, a metal hydroxide, or a metal phosphate.

23. The composition of claim 21, wherein the inorganic particle comprises aluminum oxide, aluminum oxyhydroxide, iron oxide, titanium dioxide, or silicon dioxide.

24. The composition of claim 21, wherein the inorganic particle comprises pure iron, magnetite, y—Fe₂O₃, Fe₃O₄, manganese ferrite, cobalt ferrite or nickel ferrite.

25. The composition of claim 23, wherein the inorganic particle comprises a high atomic number element, wherein the high atomic number element is gold, silver, iodine, gallium, barium, iron, gadolinium, platinum, hafnium, bismuth or combinations thereof.

26. The composition of claim 1, wherein the nanoparticle further comprises a surfactant.

27. The composition of claim 26, wherein the surfactant is a hydrophobic surfactant or a hydrophilic surfactant.

28. The composition of claim 27, wherein the hydrophobic surfactant is sorbitan monolaurate, sorbitan monopalmitate, sorbitan monostearate, sorbitan monooleate, or sorbitan trioleate.

29. The composition of claim 27, wherein the hydrophilic surfactant is a polysorbate.

30. The composition of claim 1, wherein the hydrophobic core further comprises:
a. a phosphate-terminated lipid; and
b. a surfactant.

31. The composition of claim 30, wherein the phosphate-terminated lipid is trioctylphosphine oxide (TOPO).

32. The composition of claim 30, wherein the nanoparticle further comprises an inorganic particle, and wherein the inorganic particle is coated with a capping ligand and the surfactant.

33. The composition of claim 30, wherein the surfactant is a phosphorous-terminated surfactant, a carboxylate-terminated surfactant, a sulfate-terminated surfactant, or an amine-terminated surfactant.

34. The composition of claim 30, wherein the surfactant is distearyl phosphatidic acid (DSPA).

35. The composition of claim 1, wherein the composition is lyophilized.

36. The composition of claim 1, wherein the nucleic acid is present in an amount of about 0.05, 0.1, 0.2, 0.5, 1, 5, 10, 12.5, 15, 20, 25, 50, 75, 100, 125, 150 or 200 μg.


SEQUENCE LISTING (13 sequences)

SEQ ID NO: 1 - RNA, 103 nt, synthetic construct
  source 1..103: mol_type = other RNA
ccatcctgtttttttccctttttttttttcttttttttttttttttttttttttttttttttttctcctttttttttcctctttttttccttttctttccttt

SEQ ID NO: 2 - RNA, 109 nt, synthetic construct
  source 1..109: mol_type = other RNA
ggccatcctgtttttttccctttttttttttcttttttttttttttttttttttttttttttttttctcctttttttttcctctttttttccttttctttccttttcta

SEQ ID NO: 3 - RNA, 60 nt, synthetic construct
  source 1..60: mol_type = other RNA
cccccccccccccccccccccccccccccccccccccccccccccccccccccccccccc

SEQ ID NO: 4 - RNA, 100 nt, synthetic construct
  source 1..100: mol_type = other RNA
gggggggggggggggggggggggggggggggggggggggggggggggggggggggggggggggggggggggggggggggggggggggggggggggggggg

SEQ ID NO: 5 - DNA, 165 bp, synthetic construct
  source 1..165: mol_type = other DNA
  misc_feature 1..25: note = DNA
  misc_feature 26..165: note = RNA
tgctgctgcttgcaagcagcttgataccagacaaagctgggaatagaaacttcgtattttcaaagttttctttaatatattgcaaataatgcctaaccacctagggcaggattagggttccggagttcaaccaattagtccttaatcagggcactgtatccgact

SEQ ID NO: 6 - RNA, 140 nt, synthetic construct
  source 1..140: mol_type = other RNA
agtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 7 - RNA, 32 nt, synthetic construct
  source 1..32: mol_type = other RNA
  misc_feature 1: note = 5' guanosine triphosphate
ggatcgatcgatcgttcgcgatcgatcgatcc

SEQ ID NO: 8 - RNA, 99 nt, synthetic construct
  source 1..99: mol_type = other RNA
gacgaagaccacaaaaccagataaaaaaaaaaaaaaaaaaaaaaaaaataattttttttttttttttttttttttttatctggttttgtggtcttcgtc

SEQ ID NO: 9 - RNA, 24 nt, synthetic construct
  source 1..24: mol_type = other RNA
  misc_feature 1: note = 5' guanosine triphosphate
ggacgtacgtttcgacgtacgtcc

SEQ ID NO: 10 - RNA, 87 nt, synthetic construct
  source 1..87: mol_type = other RNA
  misc_feature 1: note = 5' adenosine triphosphate
agcaaaagcagggtgacaaagacataatggatccaaacactgtgtcaagctttcaggtagattgctttctttggcatgtccgcaaac

SEQ ID NO: 11 - DNA, 122 bp, synthetic construct
  source 1..122: mol_type = other DNA
  misc_feature 1: note = 5' thymidine triphosphate
taatacgactcactataggccatcctgtttttttcccttttttttttctttttttttttttttttttttttttttttttttttctcctttttttttcctctttttttccttttctttccttt

SEQ ID NO: 12 - DNA, 138 bp, synthetic construct
  source 1..138: mol_type = other DNA
ggaactaatacgactcactataggccatcctgtttttttccctttttttttttcttttttttttttttttttttttttttttttttttctcctttttttttcctctttttttccttttctttccttttctagagttcc

SEQ ID NO: 13 - DNA, 138 bp, synthetic construct
  source 1..138: mol_type = other DNA
ggaactctagaaaaggaaagaaaaggaaaaaaagaggaaaaaaaaaggagaaaaaaaaaaaaaaaaaaaaaaaaaaaaaaaaaagaaaaaaaaaaagggaaaaaaacaggatggcctatagtgagtcgtattagttcc